(19) United States
(12) Patent Application Publication
(10) Pub. No.: US 2024/0026446 A1
(43) Pub. Date: Jan. 25, 2024

(54) SYSTEMS AND METHODS FOR SPATIAL SCREENING OF ANALYTES

(71) Applicant: ULTIMA GENOMICS, INC., Newark, CA (US)

(72) Inventors: Gilad ALMOGY, Palo Alto, CA (US); Mark PRATT, Bozeman, MT (US); Florian OBERSTRASS, Redwood City, CA (US); Ron Saar DOVER, Palo Alto, CA (US); Zohar SHIPONY, Rehovot (IL)

(21) Appl. No.: 18/480,416

(22) Filed: Oct. 3, 2023

Related U.S. Application Data
(63) Continuation of application No. PCT/US2022/024104, filed on Apr. 8, 2022.
(60) Provisional application No. 63/173,228, filed on Apr. 9, 2021.

(51) Int. Cl. C12Q 1/6874; C12N 15/10

(52) U.S. Cl. CPC C12Q 1/6874; C12N 15/1068

(57) ABSTRACT

Despite the advance of screening technology, omic-based studies with spatial resolution still requires laborious efforts, hampering the analysis of biology and disease. The present disclosure provides methods, systems, reagents, and platforms to increase the throughput of analyte screening with spatial resolution.

### CROSS-REFERENCE

This application claims the benefit of U.S. Provisional Pat. App. No. 63/173,228, filed Apr. 9, 2021, which is entirely incorporated by reference herein.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, determining transcriptomes with spatial resolution still requires laborious efforts, hampering the analysis of gene regulatory networks in spatial gene expression.

### SUMMARY

Despite the prevalence of biological sample processing systems and methods, such systems and methods may have low efficiency that can be time-intensive and wasteful of valuable resources, such as reagents. Recognized herein is a need for methods and systems for sample processing and/or analysis with high efficiency.

In an aspect, provided is a method, comprising: (a) immobilizing a plurality of beads to individually addressable locations of a first substrate to provide a barcoded substrate, wherein the plurality of beads comprises oligonucleotide molecules coupled thereto, wherein the oligonucleotide molecules comprise barcode sequences; (b) sequencing the oligonucleotide molecules on the barcoded substrate to generate indexed data that indexes the individually addressable locations of the first substrate with the barcode sequences of the oligonucleotide molecules, to provide an indexed, barcoded substrate, wherein the sequencing comprises rotating the barcoded substrate during dispensing of sequencing reagents to the barcoded substrate or during imaging of the barcoded substrate; (c) loading a biological sample to the indexed, barcoded substrate to tag analytes in the biological sample using the oligonucleotide molecules, to generate spatially tagged analytes, wherein the spatially tagged analytes comprise the barcode sequences or derivatives thereof; (d) immobilizing the spatially tagged analytes, or derivatives thereof, on a second substrate; (e) sequencing the spatially tagged analytes, or derivatives thereof, on the second substrate to generate sequencing data, wherein the sequencing comprises rotating the second substrate during dispensing of sequencing reagents to the second substrate or during imaging of the second substrate; and (f) generating spatial data of the analytes of the biological sample using the sequencing data and the indexed data.

In some embodiments, a bead of the plurality of beads comprises at least 100,000 oligonucleotide molecules. In some embodiments, the at least 100,000 oligonucleotide molecules comprise a barcode sequence of the barcode sequences that is common and unique to the bead amongst the plurality of beads.

In some embodiments, an oligonucleotide molecule of the oligonucleotide molecules comprises a capture sequence, wherein the capture sequence hybridizes with a sequence of an analyte, or derivative thereof, of the analytes. In some embodiments, the capture sequence is selected from the group consisting of: a polyT sequence, a polyG sequence, a targeted mRNA sequence, a targeted gDNA sequence, a random n-mer sequence, and a probe sequence.

In some embodiments, the biological sample comprises a tissue. In some embodiments, the analytes comprise mRNA. In some embodiments, the analytes comprise DNA. In some embodiments, the analytes comprise proteins.

In some embodiments, the first substrate comprises at least 1,000,000 individually addressable locations. In some embodiments, the first substrate comprises at least 1,000,000,000 individually addressable locations.

In some embodiments, the plurality of beads is immobilized to the individually addressable locations via electrostatic interactions.

In some embodiments, the spatially tagged analytes, or derivatives thereof, are coupled to a second plurality of beads, which second plurality of beads are immobilized to second individually addressable locations of the second substrate. In some embodiments, the second substrate comprises at least 1,000,000 second individually addressable locations. In some embodiments, the second substrate comprises at least 1,000,000,000 second individually addressable locations. In some embodiments, the second plurality of beads is immobilized to the second individually addressable locations via electrostatic interactions. In some embodiments, a second bead of the second plurality of beads comprises a colony of a spatially tagged analyte, or derivative thereof, of the spatially tagged analytes, or derivatives thereof. In some embodiments, wherein the second bead comprises at least 100,000 copies of the spatially tagged analyte, or derivative thereof.

In some embodiments, the sequencing in (b) comprises sequencing by synthesis. In some embodiments, the sequencing by synthesis comprises providing distinct nucleotide flows to the first substrate, wherein a distinct nucleotide flow of the distinct nucleotide flows comprises a single-base nucleotide mixture. In some embodiments, the single-base nucleotide mixture comprises a mixture of labeled nucleotides and non-labeled nucleotides of a single base. In some embodiments, the single-base nucleotide mixture comprises non-terminated nucleotides.

In some embodiments, the sequencing in (b) comprises rotating the barcoded substrate during dispensing of sequencing reagents. In some embodiments, the sequencing in (b) comprises rotating the barcoded substrate during imaging of the barcoded substrate. In some embodiments, the sequencing in (b) comprises rotating the barcoded substrate during dispensing of sequencing reagents and rotating the barcoded substrate during imaging of the barcoded substrate.

In some embodiments, the sequencing in (e) comprises sequencing by synthesis. In some embodiments, the sequencing by synthesis comprises providing distinct nucleotide flows to the second substrate, wherein a distinct nucleotide flow of the distinct nucleotide flows comprises a single-base nucleotide mixture. In some embodiments, the single-base nucleotide mixture comprises a mixture of labeled nucleotides and non-labeled nucleotides of a single base. In some embodiments, the single-base nucleotide mixture comprises non-terminated nucleotides.

In some embodiments, the sequencing in (e) comprises rotating the second substrate during dispensing of sequencing reagents. In some embodiments, the sequencing in (e) comprises rotating the second substrate during imaging of the second substrate. In some embodiments, the sequencing in (e) comprises rotating the second substrate during dispensing of sequencing reagents and rotating the second substrate during imaging of the second substrate.

In some embodiments, the first substrate or the second substrate is substantially planar.

In some embodiments, the method further comprises releasing the oligonucleotide molecules from the plurality of beads. In some embodiments, the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene.

In some embodiments, the method further comprises incubating the biological sample on the indexed, barcoded substrate. In some embodiments, the method further comprises fixing the biological sample. In some embodiments, the method further comprises permeabilizing the biological sample.

In another aspect, provided is a method, comprising: (a) providing (1) a first substrate comprising a plurality of beads immobilized thereto, wherein the plurality of beads comprises oligonucleotide molecules coupled thereto, wherein the oligonucleotide molecules comprise barcode sequences, and (2) indexed data comprising identities and locations of the barcode sequences on the first substrate; (b) loading a biological sample to the first substrate to tag analytes in the biological sample using the oligonucleotide molecules, to generate spatially tagged analytes, wherein the spatially tagged analytes comprise the barcode sequences or derivatives thereof; (c) immobilizing the spatially tagged analytes, or derivatives thereof, on a second substrate; (d) sequencing the spatially tagged analytes, or derivatives thereof, on the second substrate to generate sequencing data, wherein the sequencing comprises rotating the second substrate during dispensing of sequencing reagents to the second substrate or during imaging of the second substrate; and (e) generating spatial data of the analytes of the biological sample using the sequencing data and the indexed data.

In some embodiments, a bead of the plurality of beads comprises at least 100,000 oligonucleotide molecules. In some embodiments, the at least 100,000 oligonucleotide molecules comprise a barcode sequence of the barcode sequences that is common and unique to the bead amongst the plurality of beads.

In some embodiments, an oligonucleotide molecule of the oligonucleotide molecules comprises a capture sequence, wherein the capture sequence hybridizes with a sequence of an analyte, or derivative thereof, of the analytes. In some embodiments, the capture sequence is selected from the group consisting of: a polyT sequence, a polyG sequence, a targeted mRNA sequence, a targeted gDNA sequence, a random n-mer sequence, and a probe sequence.

In some embodiments, the biological sample comprises a tissue. In some embodiments, the analytes comprise mRNA. In some embodiments, the analytes comprise DNA. In some embodiments, the analytes comprise proteins.

In some embodiments, the first substrate comprises at least 1,000,000 individually addressable locations, wherein the plurality of beads are immobilized to the at least 1,000,000 individually addressable locations. In some embodiments, the first substrate comprises at least 1,000,000,000 individually addressable locations. In some embodiments, the plurality of beads is immobilized to the at least 1,000,000 individually addressable locations via electrostatic interactions.

In some embodiments, the spatially tagged analytes, or derivatives thereof, are coupled to a second plurality of beads, which second plurality of beads are immobilized to second individually addressable locations of the second substrate. In some embodiments, the second substrate comprises at least 1,000,000 second individually addressable locations. In some embodiments, the second substrate comprises at least 1,000,000,000 second individually addressable locations. In some embodiments, the second plurality of beads is immobilized to the second individually addressable locations via electrostatic interactions. In some embodiments, a second bead of the second plurality of beads comprises a colony of a spatially tagged analyte, or derivative thereof, of the spatially tagged analytes, or derivatives thereof. In some embodiments, the second bead comprises at least 100,000 copies of the spatially tagged analyte, or derivative thereof.

In some embodiments, the sequencing in (d) comprises sequencing by synthesis. In some embodiments, the sequencing by synthesis comprises providing distinct nucleotide flows to the second substrate, wherein a distinct nucleotide flow of the distinct nucleotide flows comprises a single-base nucleotide mixture. In some embodiments, the single-base nucleotide mixture comprises a mixture of labeled nucleotides and non-labeled nucleotides of a single base. In some embodiments, the single-base nucleotide mixture comprises non-terminated nucleotides.

In some embodiments, the sequencing in (d) comprises rotating the second substrate during dispensing of sequencing reagents. In some embodiments, the sequencing in (d) comprises rotating the second substrate during imaging of the second substrate. In some embodiments, the sequencing in (d) comprises rotating the second substrate during dispensing of sequencing reagents and rotating the second substrate during imaging of the second substrate.

In some embodiments, the first substrate or the second substrate is substantially planar.

In some embodiments, the method further comprises releasing the oligonucleotide molecules from the plurality of beads. In some embodiments, the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene.

In some embodiments, the method further comprises incubating the biological sample on the first substrate. In some embodiments, the method further comprises fixing the biological sample. In some embodiments, the method further comprises permeabilizing the biological sample.

In another aspect, provided is a method, comprising: (a) sequencing a first substrate comprising a plurality of beads immobilized thereto, wherein the plurality of beads comprises oligonucleotide molecules coupled thereto, wherein the oligonucleotide molecules comprise barcode sequences, to generate indexed data comprising identities and locations of the barcode sequences on the first substrate; (b) loading a biological sample to the first substrate to tag analytes in the biological sample using the oligonucleotide molecules, to generate spatially tagged analytes, wherein the spatially tagged analytes comprise the barcode sequences or derivatives thereof; (c) immobilizing the spatially tagged analytes, or derivatives thereof, on a second substrate; and (d) sequencing the second substrate to generate sequencing data, wherein the first substrate or the second substrate or both are rotated during the sequencing in (a) or (d), respectively.

In some embodiments, a bead of the plurality of beads comprises at least 100,000 oligonucleotide molecules. In some embodiments, at least 100,000 oligonucleotide molecules comprise a barcode sequence of the barcode sequences that is common and unique to the bead amongst the plurality of beads.

In some embodiments, an oligonucleotide molecule of the oligonucleotide molecules comprises a capture sequence, wherein the capture sequence hybridizes with a sequence of an analyte, or derivative thereof, of the analytes. In some embodiments, the capture sequence is selected from the group consisting of: a polyT sequence, a polyG sequence, a targeted mRNA sequence, a targeted gDNA sequence, a random n-mer sequence, and a probe sequence.

In some embodiments, the biological sample comprises a tissue. In some embodiments, the analytes comprise mRNA. In some embodiments, the analytes comprise DNA. In some embodiments, the analytes comprise proteins.

In some embodiments, the first substrate comprises at least 1,000,000 individually addressable locations immobilizing the plurality of beads. In some embodiments, the first substrate comprises at least 1,000,000,000 individually addressable locations immobilizing the plurality of beads. In some embodiments, the plurality of beads is immobilized to the at least 1,000,000 individually addressable locations via electrostatic interactions.

In some embodiments, the spatially tagged analytes, or derivatives thereof, are coupled to a second plurality of beads, which second plurality of beads are immobilized to second individually addressable locations of the second substrate. In some embodiments, the second substrate comprises at least 1,000,000 second individually addressable locations. In some embodiments, the second substrate comprises at least 1,000,000,000 second individually addressable locations. In some embodiments, the second plurality of beads is immobilized to the second individually addressable locations via electrostatic interactions. In some embodiments, a second bead of the second plurality of beads comprises a colony of a spatially tagged analyte, or derivative thereof, of the spatially tagged analytes, or derivatives thereof. In some embodiments, the second bead comprises at least 100,000 copies of the spatially tagged analyte, or derivative thereof.

In some embodiments, the sequencing in (a) comprises sequencing by synthesis. In some embodiments, the sequencing by synthesis comprises providing distinct nucleotide flows to the first substrate, wherein a distinct nucleotide flow of the distinct nucleotide flows comprises a single-base nucleotide mixture. In some embodiments, the single-base nucleotide mixture comprises a mixture of labeled nucleotides and non-labeled nucleotides of a single base. In some embodiments, the single-base nucleotide mixture comprises non-terminated nucleotides.

In some embodiments, the sequencing in (a) comprises rotating the barcoded substrate during dispensing of sequencing reagents. In some embodiments, the sequencing in (a) comprises rotating the barcoded substrate during imaging of the barcoded substrate. In some embodiments, the sequencing in (a) comprises rotating the barcoded substrate during dispensing of sequencing reagents and rotating the barcoded substrate during imaging of the barcoded substrate.

In some embodiments, the sequencing in (d) comprises sequencing by synthesis. In some embodiments, the sequencing by synthesis comprises providing distinct nucleotide flows to the second substrate, wherein a distinct nucleotide flow of the distinct nucleotide flows comprises a single-base nucleotide mixture. In some embodiments, the single-base nucleotide mixture comprises a mixture of labeled nucleotides and non-labeled nucleotides of a single base. In some embodiments, the single-base nucleotide mixture comprises non-terminated nucleotides.

In some embodiments, the sequencing in (d) comprises rotating the second substrate during dispensing of sequencing reagents. In some embodiments, the sequencing in (d) comprises rotating the second substrate during imaging of the second substrate. In some embodiments, the sequencing in (d) comprises rotating the second substrate during dispensing of sequencing reagents and rotating the second substrate during imaging of the second substrate.

In some embodiments, the first substrate or the second substrate is substantially planar.

In some embodiments, the method further comprises releasing the oligonucleotide molecules from the plurality of beads. In some embodiments, the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene.

In some embodiments, the method further comprises incubating the biological sample on the first substrate. In some embodiments, the method further comprises fixing the biological sample. In some embodiments, the method further comprises permeabilizing the biological sample.

In some embodiments, the method further comprises generating spatial data of the analytes in the biological sample using at least the indexed data and the sequencing data.

In another aspect, provided is a system, comprising: a sequencing platform configured to (i) address individually addressable locations of substrates and (ii) rotate the substrates during dispensing of sequencing reagents to the substrates or during imaging of the substrates or during both; and a first substrate comprising a plurality of beads immobilized to a plurality of individually addressable locations on the substrate, wherein the plurality of beads comprises oligonucleotide molecules coupled thereto, wherein the oligonucleotide molecules comprise barcode sequences.

In some embodiments, the system further comprises indexed data comprising identities and locations of the barcode sequences on the first substrate.

In some embodiments, the system further comprises a second substrate comprising a second plurality of individually addressable locations configured to immobilize a second plurality of beads. In some embodiments, the system further comprises the second plurality of beads.

In some embodiments, the system further comprises a reagent configured to release the oligonucleotide molecules from the plurality of beads. In some embodiments, comprises the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene.

In some embodiments, the system further comprises sequencing reagents. In some embodiments, the sequencing reagents comprise single-base nucleotide mixtures. In some embodiments, a single-base nucleotide mixture of the single-base nucleotide mixtures comprises a mixture of labeled nucleotides and non-labeled nucleotides of a single base. In some embodiments, a single-base nucleotide mixture of the single-base nucleotide mixtures comprises non-terminated nucleotides.

In some embodiments, the system further comprises amplification reagents.

In some embodiments, the system further comprises a biological sample. In some embodiments, the biological sample is a tissue. In some embodiments, the biological sample is fixed. In some embodiments, the biological sample is permeabilized. In some embodiments, the biological sample is loaded on the first substrate.

In some embodiments, a bead of the plurality of beads comprises at least 100,000 oligonucleotide molecules. In some embodiments, the at least 100,000 oligonucleotide molecules comprise a barcode sequence of the barcode sequences that is common and unique to the bead amongst the plurality of beads.

In some embodiments, an oligonucleotide molecule of the oligonucleotide molecules comprises a capture sequence, wherein the capture sequence is configured to hybridize with a sequence of an analyte, or derivative thereof, of a biological sample. In some embodiments, the capture sequence is selected from the group consisting of: a polyT sequence, a polyG sequence, a targeted mRNA sequence, a targeted gDNA sequence, a random n-mer sequence, and a probe sequence.

In some embodiments, the first substrate comprises at least 1,000,000 individually addressable locations immobilizing the plurality of beads. In some embodiments, the first substrate comprises at least 1,000,000,000 individually addressable locations immobilizing the plurality of beads.

In some embodiments, the plurality of beads is immobilized to the individually addressable locations via electrostatic interactions.

In some embodiments, the sequencing platform is configured to perform sequencing by synthesis on the substrates.

In some embodiments, the first substrate is substantially planar.

In another aspect, provided is a kit, comprising: a first substrate comprising a plurality of beads immobilized to a plurality of individually addressable locations on the substrate, wherein the plurality of beads comprises oligonucleotide molecules coupled thereto, wherein the oligonucleotide molecules comprise barcode sequences; indexed data comprising identities and locations of the barcode sequences on the first substrate; and a second substrate comprising a second plurality of individually addressable locations configured to immobilize a second plurality of beads.

In some embodiments, the kit further comprises the second plurality of beads not immobilized to the second substrate.

In some embodiments, the kit further comprises a reagent configured to release the oligonucleotide molecules from the plurality of beads. In some embodiments, the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene.

In some embodiments, the kit further comprises sequencing reagents. In some embodiments, the sequencing reagents comprise single-base nucleotide mixtures. In some embodiments, a single-base nucleotide mixture of the single-base nucleotide mixtures comprises a mixture of labeled nucleotides and non-labeled nucleotides of a single base. In some embodiments, a single-base nucleotide mixture of the single-base nucleotide mixtures comprises non-terminated nucleotides.

In some embodiments, the kit further comprises amplification reagents.

In some embodiments, the kit further comprises a biological sample. In some embodiments, the biological sample is a tissue. In some embodiments, the biological sample is fixed. In some embodiments, the biological sample is permeabilized. In some embodiments, the biological sample is loaded on the first substrate.

In some embodiments, a bead of the plurality of beads comprises at least 100,000 oligonucleotide molecules. In some embodiments, the at least 100,000 oligonucleotide molecules comprise a barcode sequence of the barcode sequences that is common and unique to the bead amongst the plurality of beads.

In some embodiments, an oligonucleotide molecule of the oligonucleotide molecules comprises a capture sequence, wherein the capture sequence is configured to hybridize with a sequence of an analyte, or derivative thereof, of a biological sample. In some embodiments, the capture sequence is selected from the group consisting of: a polyT sequence, a polyG sequence, a targeted mRNA sequence, a targeted gDNA sequence, a random n-mer sequence, and a probe sequence.

In some embodiments, the first substrate comprises at least 1,000,000 individually addressable locations. In some embodiments, the first substrate comprises at least 1,000,000,000 individually addressable locations. In some embodiments, the second substrate comprises at least 1,000,000 individually addressable locations. In some embodiments, the second substrate comprises at least 1,000,000,000 individually addressable locations.

In some embodiments, the plurality of beads is immobilized to the plurality of individually addressable locations via electrostatic interactions.

In some embodiments, the first substrate or the second substrate is substantially planar. In some embodiments, the first substrate and the second substrate are substantially planar. In some embodiments, the first substrate and the second substrate are substantially identical in size.

In another aspect, provided is a kit, comprising: a substrate comprising a plurality of beads immobilized to a plurality of individually addressable locations on the substrate, wherein the plurality of beads comprises oligonucleotide molecules coupled thereto, wherein the oligonucleotide molecules comprise barcode sequences, wherein the oligonucleotide molecules are releasable from the plurality of beads by one or more of the release mechanisms selected from the group consisting of: (a) providing an enzyme mix comprising an enzyme configured to cleave or digest a cleavage site, wherein the oligonucleotide molecules comprises the cleavage site; (b) providing biotin, wherein the oligonucleotide molecules are conjugated to a desthiobiotin moiety, wherein the bead comprises a streptavidin moiety bound to the desthiobiotin moiety; (c) providing an enzyme mix comprising an enzyme configured to cleave or digest a cleavage site and UV light, wherein the oligonucleotide molecules comprises azobenzene and the cleavage site; and (d) providing UV light, wherein the oligonucleotide molecules are conjugated azobenzene, wherein the plurality of beads comprises an alpha-cyclodextrine (a-CD) moiety bound to the azobenzene.

In some embodiments, the kit further comprises indexed data comprising identities and locations of the barcode sequences on the substrate.

In some embodiments, the kit further comprises a second substrate comprising a second plurality of individually addressable locations configured to immobilize a second plurality of beads. In some embodiments, the kit further comprises the second plurality of beads.

In some embodiments, the kit further comprises a reagent configured to release the oligonucleotide molecules from the plurality of beads. In some embodiments, the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene.

In some embodiments, the kit further comprises sequencing reagents. In some embodiments, the sequencing reagents comprise single-base nucleotide mixtures. In some embodiments, a single-base nucleotide mixture of the single-base nucleotide mixtures comprises a mixture of labeled nucleotides and non-labeled nucleotides of a single base. In some embodiments, a single-base nucleotide mixture of the single-base nucleotide mixtures comprises non-terminated nucleotides.

In some embodiments, the kit further comprises amplification reagents.

In some embodiments, the kit further comprises a biological sample. In some embodiments, the biological sample is a tissue. In some embodiments, the biological sample is fixed. In some embodiments, the biological sample is permeabilized. In some embodiments, the biological sample is loaded on the first substrate.

In some embodiments, a bead of the plurality of beads comprises at least 100,000 oligonucleotide molecules. In some embodiments, the at least 100,000 oligonucleotide molecules comprise a barcode sequence of the barcode sequences that is common and unique to the bead amongst the plurality of beads.

In some embodiments, an oligonucleotide molecule of the oligonucleotide molecules comprises a capture sequence, wherein the capture sequence is configured to hybridize with a sequence of an analyte, or derivative thereof, of a biological sample. In some embodiments, the capture sequence is selected from the group consisting of: a polyT sequence, a polyG sequence, a targeted mRNA sequence, a targeted gDNA sequence, a random n-mer sequence, and a probe sequence.

In some embodiments, the substrate comprises at least 1,000,000 individually addressable locations. In some embodiments, the substrate comprises at least 1,000,000,000 individually addressable locations.

In some embodiments, the plurality of beads is immobilized to the individually addressable locations via electrostatic interactions.

In some embodiments, the substrate is substantially planar.

In another aspect, provided herein is a method comprising providing a substrate comprising a cell or tissue sample thereon; subjecting the substrate to rotation to distribute one or more reagents to the cell or tissue sample; and using a detector in optical communication with the cell or tissue sample on the substrate to detect an analyte of the cell or tissue sample.

In another aspect, provided herein is a method comprising providing a substrate comprising a cell or tissue sample thereon; distributing one or more reagents to the cell or tissue sample; and using a detector in optical communication with the cell or tissue sample on the substrate to detect an analyte of the cell or tissue sample, during rotation of the substrate.

In some embodiments, the analyte is within the cell or tissue sample. In some embodiments, the analyte comprises a nucleic acid molecule, a polypeptide molecule, a protein molecule, a carbohydrate molecule, a lipid molecule, any derivative thereof, or any combination thereof. In some embodiments, the analyte comprises the nucleic acid molecule, any derivative thereof, or any combination thereof. In some embodiments, the nucleic acid molecule comprises ribonucleic acid (RNA) or derivative thereof. In some embodiments, the RNA comprises a messenger RNA (mRNA). In some embodiments, the nucleic acid molecule comprises deoxyribonucleic acid (DNA) or derivative thereof. In some embodiments, the analyte comprises a polypeptide molecule or derivative thereof. In some embodiments, the analyte comprises a protein molecule or derivative thereof. In some embodiments, the analyte comprises a carbohydrate molecule. In some embodiments, the carbohydrate comprises a monosaccharide, a polysaccharide or a lignin. In some embodiments, the carbohydrate comprises a monosaccharide. In some embodiments, the carbohydrate comprises a polysaccharide. In some embodiments, the carbohydrate comprises a lignin.

In some embodiments, the one or more reagents comprises a probe configured to detect the analyte. In some embodiments, the probe comprises one or more nucleotides. In some embodiments, the probe comprises one or more nucleic acid molecules. In some embodiments, the probe comprises a padlock probe. In some embodiments, the probe comprises a barcode sequence. In some embodiments, the one or more reagents comprises a second probe that pairs with the probe. In some embodiments, the probe hybridizes to, or adjacent to, the analyte. In some embodiments, the probe hybridizes to, or adjacent to, a derivative of the analyte. In some embodiments, the probe ligates to, or adjacent to, the analyte. In some embodiments, the probe ligates to, or adjacent to, a derivative of the analyte. In some embodiments, the derivative is an amplification product of the analyte. In some embodiments, the derivative is an extension product of the analyte. In some embodiments, the derivative is a circularization product of the analyte. In some embodiments, the probe comprises an optical moiety. In some embodiments, the probe comprises a fluorescent dye.

In some embodiments, the one or more reagents comprises an additional probe configured to detect an additional analyte, wherein the additional probe comprises an additional fluorescent dye. In some embodiments, the fluorescent dye and the additional fluorescent dye are different. In some embodiments, the fluorescent dye and the additional fluorescent dye are the same. In some embodiments, the probe and the additional probe are different. In some embodiments, the probe and the additional probe are the same. In some embodiments, the analyte and the additional analyte are different types of analyte. In some embodiments, the analyte and the additional analyte are the same type of analyte. In some embodiments, the method further comprises, subsequent to (c), (i) delivering an additional probe to the cell or tissue sample, wherein the additional probe is configured to detect an additional analyte, and (ii) using the detector to detect the additional analyte of the cell or tissue sample. In some embodiments, wherein (c) comprises identifying (1) an identity and (2) a location of the analyte in the cell or tissue sample.

In some embodiments, the location is a two dimensional (2D) location. In some embodiments, the location is a three dimensional (3D) location. In some embodiments, the identity comprises a sequence of the analyte. In some embodiments, the identity comprises a type of the analyte. In some embodiments, the substrate is substantially planar. In some embodiments, the substrate is patterned. In some embodiments, the substrate is patterned via surface chemistry. In some embodiments, the substrate has a shape, wherein the shape comprises a regular polygon or an irregular polygon. In some embodiments, the shape comprises the regular polygon. In some embodiments, the regular polygon comprises a rectangle. In some embodiments, the substrate has a shape, wherein the shape comprises a circle or an oval.

In some embodiments, the substrate comprises an array of a plurality of individually addressable locations, wherein in (a) the cell or tissue sample is immobilized to one or more individually addressable locations of the plurality of individually addressable locations. In some embodiments, the array comprises at least 1,000 individually addressable locations. In some embodiments, the array comprises at least 100,000 individually addressable locations. In some embodiments, the array comprises at least 10,000,000 individually addressable locations. In some embodiments, the array comprises at least 50,000,000,000 individually addressable locations. In some embodiments, wherein (a) comprises providing the substrate comprising a plurality of cells or tissue samples thereon. In some embodiments, the plurality of cells or tissue samples comprises at least about 100 to at least about 1,000,000 cells or tissue samples.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example sequencing workflow, as disclosed herein.

FIG. 2 illustrates examples of individually addressable locations distributed on substrates, as described herein.

FIGS. 3A-3G illustrate different examples of cross-sectional surface profiles of a substrate, as described herein.

FIG. 4 shows an example coating of a substrate with a hexagonal lattice of beads, as described herein.

FIGS. 5A-5B illustrate example systems and methods for loading a sample or a reagent onto a substrate, as described herein.

FIG. 6 illustrates a computerized system for sequencing a nucleic acid molecule.

FIGS. 7A-7C illustrate multiplexed stations in a sequencing system.

FIG. 8 illustrates an example method for spatially analyzing a biological sample using molecular labels with substrate-based sample processing systems.

FIGS. 9A-9B illustrate an example method for generating and using a bead comprising molecular labels.

FIGS. 9C-9D illustrate example release mechanisms of oligonucleotide molecules from beads.

FIG. 9E illustrates an example of an additional barcode bead design.

FIG. 10 shows an example substrate with probes at individually addressable locations for capturing analytes from different spatial locations.

FIG. 11 shows an example method of using an array with probes for capturing endogenous analytes with spatial resolution.

FIG. 12A shows an example padlock probe.

FIG. 12B shows an example padlock probe set.

FIG. 13 shows another example probe for detecting an analyte.

FIG. 14 shows an example pseudo-fluorescent labeling process.

FIG. 15 illustrates a computer system that is programmed or otherwise configured to implement methods provided herein.

FIG. 16 shows an example of an image generated by imaging a substrate with an analyte immobilized thereto.

FIGS. 17A and 17B illustrate exemplary data from processed images.

FIG. 18A shows a plot of aligned genomic reads. FIG. 18B shows aligned coverage distribution over a reference genome.

FIG. 19 illustrates a schematic for subjecting a reaction space to electrophoresis.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule and where the single-stranded portion is at a 3′ or 5′ end of the same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, kb, 30 kb, 40 kb, 50 kb, 100 kb, 200 kb, 300 kb, 400 kb, 500 kb, 1 megabase (Mb), Mb, 100 Mb, 1 gigabase or more. A nucleic acid can comprise a sequence of four natural nucleotide bases: adenine (A); cytosine (C); guanine (G); and thymine (T) (or uracil (U) instead of thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:

(e.g., [A T G C A T G C A T G A T G A T G A T
G C A T G C]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow order, consecutively or non-consecutively. Accordingly, the term “flow cycle order,” as used herein, generally refers to an ordering of flow cycles within the flow order, and can be expressed in units of flow cycles. For example, where [A T G C] is identified as a 1^{st} flow cycle, and [A T G] is identified as a 2^{nd} flow cycle, the flow order of [A T G C A T G C A T G A T G A T G A T G C A T G C] may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle]. Alternatively or in addition, the flow cycle order may be described as [cycle 1, cycle, 2, cycle 3, cycle 4, cycle 5, cycle 6], where cycle 1 is the 1^{st} flow cycle, cycle 2 is the 1st flow cycle, cycle 3 is the 2^{nd} flow cycle, etc.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C.C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity, and as such sequences would generally be considered “identical.”

The term “detector,” as used herein, generally refers to a device that is capable of detecting a signal, including a signal indicative of the presence or absence of one or more incorporated nucleotides or fluorescent labels. The detector may simultaneously or substantially simultaneously detect multiple signals. The detector may detect the signal in real-time during, substantially during a biological reaction, such as a sequencing reaction (e.g., sequencing during a primer extension reaction), or subsequent to a biological reaction. In some cases, a detector can include optical and/or electronic components that can detect signals. Non-limiting examples of detection methods, for which a detector is used, include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area where the scanning is electronically synchronized to the image of an object in relative motion. A continuous area scanning detector may comprise a time delay and integration (TDI) charge coupled device (CCD), Hybrid TDI, complementary metal oxide semiconductor (CMOS) pseudo TDI device, or TDI line-scan camera.

### Sample Processing Methods

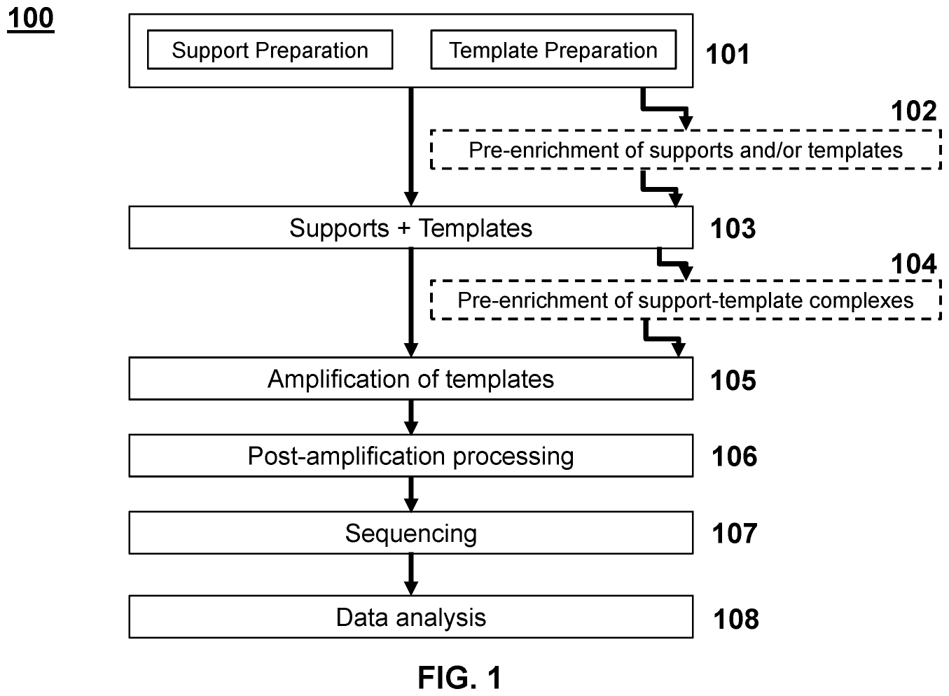

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be prepared and/or provided (101) to be compatible with downstream sequencing operations (e.g., 107). A support (e.g., bead) may be used to help facilitate sequencing of a template nucleic acid on a substrate. The support may help immobilize a template nucleic acid to a substrate, such as when the template nucleic acid is coupled to the support, and the support is in turn immobilized to the substrate. The support may further function as a binding entity to retain molecules of a colony of the template nucleic acid (e.g., copies comprising identical or substantially identical sequences as the template nucleic acid) together for any downstream processing, such as for sequencing operations. This may be particularly useful in distinguishing a colony from other colonies (e.g., on other supports) and generating amplified sequencing signals for a template nucleic acid sequence.

A support that is prepared and/or provided may comprise an oligonucleotide comprising one or more functional nucleic acid sequences. For example, the support may comprise a capture sequence configured to capture or be coupled to a template nucleic acid (or processed template nucleic acid). For example, the support may comprise the capture sequence, a primer sequence, a barcode sequence, a sample index sequence, a unique molecular identifier (UMI), a flow cell adapter sequence, an adapter sequence, a binding sequence for any molecule (e.g., splint, primer, template nucleic acid, capture sequence, etc.), or any other functional sequence useful for a downstream operation, or any combination thereof. The oligonucleotide may be single-stranded, double-stranded, or partially double-stranded.

A support may comprise one or more capture entities, where a capture entity is configured for capture by a capturing entity. A capture entity may be coupled to an oligonucleotide coupled to the support. A capture entity may be coupled to the support. For example, the capturing entity may comprise streptavidin (SA) when the capture moiety comprises biotin. In another example, the capturing entity may comprise a complementary capture sequence when the capture entity comprises a capture sequence (e.g., a capture oligonucleotide that is complementary to the complementary capture sequence). In another example, the capturing entity may comprise an apparatus, system, or device configured to apply a magnetic field when the capture entity comprises a magnetic particle. In another example, the capturing entity may comprise an apparatus, system, or device configured to apply an electrical field when the capture entity comprises a charged particle. In some instances, the capturing entity may comprise one or more other mechanisms configured to capture the capture entity. A capture entity and capturing entity may bind, couple, hybridize, or otherwise associate with each other. The association may comprise formation of a covalent bond, non-covalent bond, and/or releasable bond (e.g., cleavable bond that is cleavable upon application of a stimulus). In some cases, the association may not form any bond. For example, the association may increase a physical proximity (or decrease a physical distance) between the capturing entity and capture entity. In some instances, a single capture entity may be capable of associating with a single capturing entity. Alternatively, a single capture entity may be capable of associating with multiple capturing entities. Alternatively or in addition, a single capturing entity may be capable of associating with multiple capture entities. The capture entity may be capable of linking to a nucleotide. Chemically modified bases comprising biotin, an azide, cyclooctyne, tetrazole, and a thiol, and many others are suitable as capture entities. The capture entity/capturing entity pair may be any combination. The pair may include, but is not limited to, biotin/streptavidin, azide/cyclooctyne, and thiol/maleimide. It will be appreciated that either of the pair may be used as either the capture entity or the capturing entity. In some instances, the capturing entity may comprise a secondary capture entity, for example, for subsequent capture by a secondary capturing entity. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic bead) and the secondary capturing entity can comprise a magnetic system (e.g., magnet, apparatus, system, or device configured to apply a magnetic field, etc.). In some instances, the secondary capture entity can comprise a charged particle (e.g., charged bead carrying an electrical charge) and the secondary capturing entity can comprise an electrical system (e.g., magnet, apparatus, system, or device configured to apply an electric field, etc.).

A support may comprise one or more cleaving moieties. The cleavable moiety may be part of or attached to an oligonucleotide coupled to the support. The cleavable moiety may be coupled to the support. A cleavable moiety may comprise any useful cleavable or excisable moiety that can be used to cleave an oligonucleotide (or portion thereof) from the support. For example, the cleavable moiety may comprise a uracil, a ribonucleotide, or other modified nucleotide that is excisable or cleavable using an enzyme (e.g., UDG, RNAse, endonuclease, exonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethyleneglycol spacer (e.g., Spacer 18), or combinations or analogs thereof. The cleavable moiety may comprise a photocleavable moiety. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APE1, MspJI, etc.). Alternatively, or in addition to, the cleavable moiety may be cleavable using one or more stimuli, e.g., photo-stimulus, chemical stimulus, thermal stimulus, etc.

In some examples, a single support comprises copies of a single species of oligonucleotide, which are identical or substantially identical to each other. In some examples, a single support comprises copies of at least two species of oligonucleotides (e.g., comprising different sequences). For example, a single support may comprise a first subset of oligonucleotides configured to capture a first adapter sequence of a template nucleic acid and a second subset of oligonucleotides configured to capture a second adapter sequence of a template nucleic acid.

In some examples, a population of a single species of supports may be prepared and/or provided, where all supports within a species of supports is identical (e.g., has identical oligonucleotide composition (e.g., sequence), etc.). In some examples, a population of multiple species of supports may be prepared and/or provided. For example, a population of supports may be prepared to comprise a plurality of unique support species, where each unique support species comprises a primer sequence unique to said support species. When attaching template nucleic acids to supports, only a template nucleic acid comprising a given adapter sequence compatible with (e.g., at least partially complementary to) a given primer sequence may be capable of attaching to a given support of a support species comprising the given primer sequence. In another example, a population of supports may be prepared, such that each unique support species comprises a plurality of primer sequences (e.g., a pair of primer sequences) unique to said support species. In some embodiments, the systems and methods disclosed herein can include a population of supports that comprise two, three, four, five, six, seven, eight, nine, ten or more unique support species. Each unique support species can comprise a unique primer sequence that allows selective interactions between the respective support species with an intended binding partner (e.g., a complementary nucleic acid sequence within an adapter region of a template nucleic acid or an intermediary primer sequence which can subsequently bind to a complementary nucleic acid sequence within an adapter region of a sample nucleic acid). A population of multiple species of supports may be prepared by first preparing distinct populations of a single species of supports, all different, and mixing such distinct populations of single species of supports to result in the final population of multiple species of supports. A concentration of the different support species within the final mixture may be adjusted accordingly. Devices, systems, methods, compositions, and kits for preparing and using support species are described in further detail in Patent Pub. Nos. US20220042072A1 and WO2022040557A2, each of which is entirely incorporated herein by reference for all purposes.

A template nucleic acid may include an insert sequence sourced from a biological sample. In some cases, the insert sequence may be derived from a larger nucleic acid in the biological sample (e.g., an endogenous nucleic acid), or reverse complement thereof, for example by fragmenting, transposing, and/or replicating from the larger nucleic acid. The template nucleic acid may be derived from any nucleic acid of the biological sample and result from any number of nucleic acid processing operations, such as but not limited to fragmentation, degradation or digestion, transposition, ligation, reverse transcription, extension, etc. A template nucleic acid that is prepared and/or provided may comprise one or more functional nucleic acid sequences. In some cases, the one or more functional nucleic acid sequences may be disposed at one end of the insert sequence. In some cases, the one or more functional nucleic acid sequences may be separated and disposed at both ends of an insert sequence, such as to sandwich the insert sequence. In some cases, a nucleic acid molecule comprising the insert sequence, or complement thereof, may be ligated to one or more adapter oligonucleotides that comprise such functional nucleic acid sequence(s). In some cases, a nucleic acid molecule comprising the insert sequence, or complement thereof, may be hybridized to a primer comprising such functional nucleic acid sequence(s) and extended to generate a template nucleic acid comprising such functional nucleic acid sequence(s). In some cases, a nucleic acid molecule comprising the insert sequence, or complement thereof, may be hybridized to a primer comprising one or more functional nucleic acid sequence(s) and extended to generate an intermediary molecule, and the intermediary molecule hybridized to a primer comprising additional functional nucleic acid sequence(s) and extended, and so on for any number of extension reactions, to generate a template nucleic acid comprising one or more functional nucleic acid sequence(s). For example, the template nucleic acid may comprise an adapter sequence configured to be captured by a capture sequence on an oligonucleotide coupled to a support. For example, the template nucleic acid may comprise a capture sequence, a primer sequence, a barcode sequence, a sample index sequence, a unique molecular identifier (UMI), a flow cell adapter sequence, the adapter sequence, a binding sequence for any molecule (e.g., splint, primer, template nucleic acid, capture sequence, etc.), or any other functional sequence useful for a downstream operation, or any combination thereof. The template nucleic acid may be single-stranded, double-stranded, or partially double-stranded.

A template nucleic acid may comprise one or more capture entities that are described elsewhere herein. In some cases, in the workflow, only the supports comprise capture entities and the template nucleic acids do not comprise capture entities. In other cases, in the workflow, only the template nucleic acids comprise capture entities and the supports do not comprise capture entities. In other cases, both the template nucleic acids and the supports comprise capture entities. In other cases, neither the supports nor the template nucleic acids comprises capture entities.

A template nucleic acid may comprise one or more cleaving moieties that are described elsewhere herein. In some cases, in the workflow, only the supports comprise cleavable moieties and the template nucleic acids do not comprise cleavable moieties. In other cases, in the workflow, only the template nucleic acids comprise cleavable moieties and the supports do not comprise cleavable moieties. In other cases, both the template nucleic acids and the supports comprise cleavable moieties. In other cases, neither the supports nor the template nucleic acids comprises cleavable moieties. A cleavable moiety may be strategically placed based on a desired downstream amplification workflow, for example.

In some examples, a library of insert sequences are processed to provide a population of template sequences with identical configurations, such as with identical sequences and/or locations of one or more functional sequences. For example, a population of template sequences may comprise a plurality of nucleic acid molecules each comprising an identical first adapter sequence ligated to a same end. In some examples, a library of insert sequences are processed to provide a population of template sequences with varying configurations, such as with varying sequences and/or locations of one or more functional sequences. For example, a population of template sequences may comprise a first subset of nucleic acid molecules each comprising an identical first adapter sequence at a first end, and a second subset of nucleic acid molecules each comprising an identical second adapter sequence at the second end, where the second adapter sequence is different form the first adapter sequence. In some instances, a population of template sequences with varying configurations (e.g., varying adapter sequences) may be used in conjunction with a population of multiple species of supports, such as to reduce polyclonality problems during downstream amplification. A population of multiple configurations of template nucleic acids may be prepared by first preparing distinct populations of a single configuration of template nucleic acids, all different, and mixing such distinct populations of single configurations of template nucleic acids to result in the final population of multiple configurations of template nucleic acids. A concentration of the different configurations of template nucleic acids within the final mixture may be adjusted accordingly.

Optionally, the supports and/or template nucleic acids may be pre-enriched (102). For example, a support comprising a distinct oligonucleotide sequence is isolated from a mixture comprising support(s) that do not have the distinct oligonucleotide sequence. Alternatively, a support population may be provided to comprise substantially uniform supports, where each support comprises an identical surface primer molecule immobilized thereto. For example, template nucleic acids comprising a distinct configuration (e.g., comprising a particular adapter sequence) is isolated from a mixture comprising template nucleic acids that do not have the distinct configuration. Alternatively, a template nucleic acid population may be provided to comprise substantially uniform configurations. In some cases, the capture entit(ies) on the supports and/or template nucleic acids are used for pre-enrichment.

Subsequent to preparation of the supports and template nucleic acids, the two may be attached (103). A template nucleic acid may be coupled to a support via any method(s) that results in a stable association between the template nucleic acid and the support. For example, the template nucleic acid may hybridize to an oligonucleotide on the support. In another example, the template nucleic acid may hybridize to one or more intermediary molecules, such as a splint, bridge, and/or primer molecule, which hybridizes to an oligonucleotide on the support. Alternatively or in addition, a template nucleic acid may be ligated to one or more nucleic acids on or coupled to the support. Alternatively or in addition, a template nucleic acid may be hybridized to an oligonucleotide on a support, which oligonucleotide comprises a primer sequence, and subsequent extension form the primer sequence is performed. Once attached, a plurality of support-template complexes may be generated.

Optionally, support-template complexes may be pre-enriched (104), wherein a support-template complex is isolated from a mixture comprising support(s) and/or template nucleic acid(s) that are not attached to each other. In some cases, the capture entit(ies) on the supports and/or template nucleic acids are used for pre-enrichment.

Subsequent to attachment of the template nucleic acid molecule to the support, the template nucleic acids may be subjected to amplification reactions (105) to generate a plurality of amplification products immobilized to the support. For example, such amplification reactions may comprise performing polymerase chain reaction (PCR) or any other amplification methods described herein, including but not limited to emulsion PCR (ePCR or emPCR), isothermal amplification (e.g., recombinase polymerase amplification (RPA)), bridge amplification, template walking, etc. In some cases, amplification reactions can occur while the support is immobilized to a substrate. In other cases, amplification reactions can occur off the substrate, such as in solution, or on a different surface or platform. In some cases, amplification reactions can occur in isolated reaction volumes, such as within multiple droplets in an emulsion during emulsion PCR (ePCR or emPCR), or in wells. Emulsion PCR methods are described in further detail in Patent Pub. Nos. US20220042072A1 and WO2022040557A2, each of which is entirely incorporated by reference herein.

Subsequent to amplification, the supports (e.g., comprising the template nucleic acids) may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pat. No. 10,900,078 and Patent Pub. Nos. US20210079464A1 and WO2022040557A2, each of which is entirely incorporated by reference herein. For example, an on-substrate enrichment procedure may immobilize only the positive supports onto the substrate surface to isolate the positive supports. In some instances, the positive supports may be immobilized to desired locations on the substrate surface (e.g., individually addressable locations), as distinguished from undesired locations (e.g., spacers between the individually addressable locations). In some instances, positive supports and/or negative supports may be processed to selectively remove unamplified surface primers (on the support(s)), such that a resulting positive support retains the template nucleic acid molecule, and a resulting negative support is stripped of the unamplified surface primers. Subsequently, the template nucleic acid(s) on the positive supports may be used to enrich for the positive supports, e.g., by capturing the template nucleic acids.

Subsequent to post-amplification processing, the template nucleic acids may be subject to sequencing (107). The template nucleic acid(s) may be sequenced while attached to the support. Alternatively, the template nucleic acid molecules may be free of the support when sequenced and/or analyzed. In some instances, the template nucleic acids may be sequenced while attached to the support which is immobilized to a substrate. Examples of substrate-based sample processing systems are described elsewhere herein. Any sequencing method described elsewhere herein may be used. In some cases, sequencing by synthesis (SBS) is performed.

In one example (Example A), an SBS method comprises flowing nucleotide reagents according to a flow order comprising a repeat of one 4-base flow (e.g., [A/T/G/C]), where each nucleotide is reversibly terminated (e.g., dideoxynucleotide), and where each base is labeled with a different dye (yielding different optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of each base can be detected by interrogating the different dyes in 4 channels. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example B), an SBS method comprises flowing nucleotide reagents according to a flow order comprising a repeat of a flow cycle of 4 single base flows (e.g., [A T G C]), where each nucleotide is reversibly terminated, and where each base is labeled with a same dye (yielding same frequency optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of the particular base in that flow can be detected by interrogating the wavelength of the dye. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example C), an SBS method comprises flowing nucleotide reagents according to a flow order comprising a repeat of a flow cycle of 4 single base flows (e.g., [A T G C]), where each nucleotide is not terminated, and where each base is labeled with a same dye (yielding same frequency optical signals). With each flow, other sequencing reagents, e.g., sequencing primer, polymerase, buffer, etc. are present to provide sufficient conditions for incorporation of the labeled nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example D), an SBS method comprises flowing nucleotide reagents according to a flow order comprising a repeat of a flow cycle of 4 single base flows (e.g., [A T G C]), where each nucleotide is not terminated, and where only a fraction of the bases in each flow (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals). With each flow, other sequencing reagents, e.g., sequencing primer, polymerase, buffer, etc. are present to provide sufficient conditions for incorporation of the nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example E), an SBS method comprises flowing nucleotide reagents according to a flow order comprising a repeat of a flow cycle of 8 single base flows, with each of the 4 canonical base types flowed twice consecutively within the flow cycle, (e.g., [A A T T G G C C]), where each nucleotide is not terminated, and where only a fraction of the bases in every other flow in the flow cycle (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals) and the nucleotides in the alternating other flow is unlabeled. With each flow, other sequencing reagents, e.g., sequencing primer, polymerase, buffer, etc. are present to provide sufficient conditions for incorporation of the nucleotide into a growing strand hybridized to a template nucleic acid. After one or both of the flows for each canonical base type, an incorporation event or lack thereof of the particular base in that flow can be detected by interrogating the wavelength of the dye. Because the nucleotides are not terminated, if the growing strand is extending through a homopolymer region (e.g., polyT region) of the template nucleic acid, multiple nucleotides may be incorporated during one flow. A first flow of a canonical base type (e.g., A) followed by a second flow of the same canonical base type (e.g., A) may help facilitate completion of incorporation reactions across each growing strand such as to reduce phasing problems. After each or one or more detection events, the labels may be removed (e.g., dyes are cleaved) to reduce signal noise for the next detection.

Labeled nucleotides may comprise a dye, fluorophore, or quantum dot. Non-limiting examples of dyes include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto 390, 425, 465, 488, 495, 532, 565, 594, 633, 647, 647N, 665, 680 and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BHI-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q. In some cases, the label may be one with linkers. For instance, a label may have a disulfide linker attached to the label. Non-limiting examples of such labels include Cy5-azide, Cy-2-azide, Cy-3-azide, Cy-3.5-azide, Cy5.5-azide and Cy-7-azide. In some cases, a linker may be a cleavable linker. In some cases, the label may be a type that does not self-quench or exhibit proximity quenching. Non-limiting examples of a label type that does not self-quench or exhibit proximity quenching include Bimane derivatives such as Monobromobimane. Alternatively, the label may be a type that self-quenches or exhibits proximity quenching. Non-limiting examples of such labels include Cy5-azide, Cy-2-azide, Cy-3-azide, Cy-3.5-azide, Cy5.5-azide and Cy-7-azide. In some instances, a blocking group of a reversible terminator may comprise the dye. The labels (e.g., dyes, fluorophores, etc.) described herein may be coupled to any probe, such as a nucleotide or oligonucleotide.

It will be appreciated that the combinations of termination states on the nucleotides, label types (e.g., types of dye or other detectable moiety), fraction of labeled nucleotides within a flow, type of nucleotide bases in each flow, type of nucleotide bases in each flow cycle, and/or the order of flows in a flow cycle and/or flow order, other than enumerated in Examples A-E, can be varied for different SBS methods.

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from. In some cases, the sequencing reads may be processed to generate spatial data.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed.

The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates and detectors that can be used in the sample processing system are described in further detail in Patent Pub. Nos. US20200326327A1, US20210354126A1, and US20210079464A1, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 micrometers (μm), at least 200 μm, at least 500 μm, at least 1 mm, at least 2 millimeters (mm), at least 5 mm, at least 10 mm, or more. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least 1 mm, at least 2 mm, at least 5 mm, at least 10 mm, at least 20 mm, at least 50 mm, at least 100 mm, at least 200 mm, at least 500 mm, at least 1,000 mm, or more.

One or more surfaces of the substrate may be exposed to a surrounding open environment, and accessible from such surrounding open environment. For example, the array may be exposed and accessible from such surrounding open environment. In some cases, as described elsewhere herein, the surrounding open environment may be controlled and/or confined in a larger controlled environment.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing.

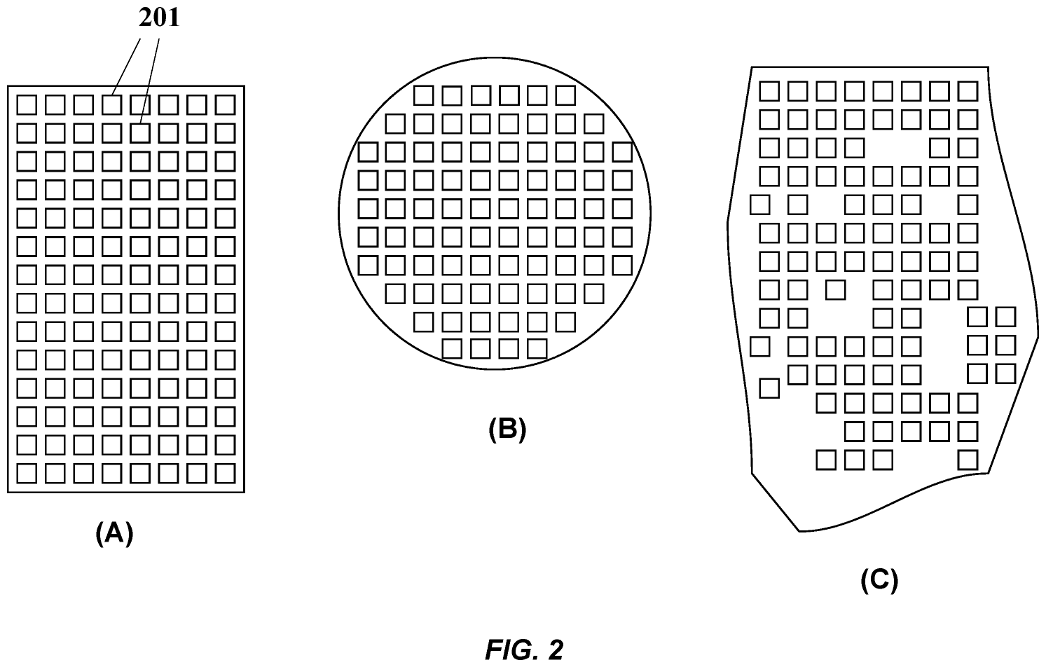

The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays. The substrate may have any number of individually addressable locations, for example, at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more individually addressable locations. The substrate may have a number of individually addressable locations that is within a range defined by any two of the preceding values.

Each individually addressable location may have the general shape or form of a circle, pit, bump, rectangle, or any other shape or form (e.g., polygonal, non-polygonal). A plurality of individually addressable locations can have uniform shape or form, or different shapes or forms. An individually addressable location may have any size. In some cases, an individually addressable location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 μm², about 3.75 μm², about 4 μm², about 4.25 μm², about 4.5 μm², about 4.75 μm², about 5 μm², about 5.5 μm², about 6 μm², or more. An individually addressable location may have an area that is within a range defined by any two of the preceding values. An individually addressable location may have an area that is less than about 0.1 μm² or greater than about 6 μm².

The individually addressable locations may be distributed on a substrate with a pitch determined by the distance between the center of a first location and the center of the closest or neighboring individually addressable location. Locations may be spaced with a pitch of about 0.1 micron (μm), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some cases, the locations may be positioned with a pitch that is within a range defined by any two of the preceding values. The locations may be positioned with a pitch of less than about 0.1 μm or greater than about 10 μm. In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). In some cases, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents, such as via the support. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.). In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location.

A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

In some cases, an individually addressable location may comprise a distinct surface chemistry. The distinct surface chemistry may distinguish between different addressable locations. The distinct surface chemistry may distinguish an individually addressable location from a surrounding location on the substrate. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object due to different surface chemistries. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. For example, a first location type or region type may comprise a positively charged surface chemistry and a second location type or region type may comprise a negatively charged surface chemistry. In another example, a first location type or region type may comprise a hydrophobic surface chemistry and a second location type or region type may comprise a hydrophilic surface chemistry. In another example, a first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., a reference bead that always emits a detectable signal during detection), and the like, and the individually addressable locations may be indexed with reference to such demarcations. As will be appreciated, a combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

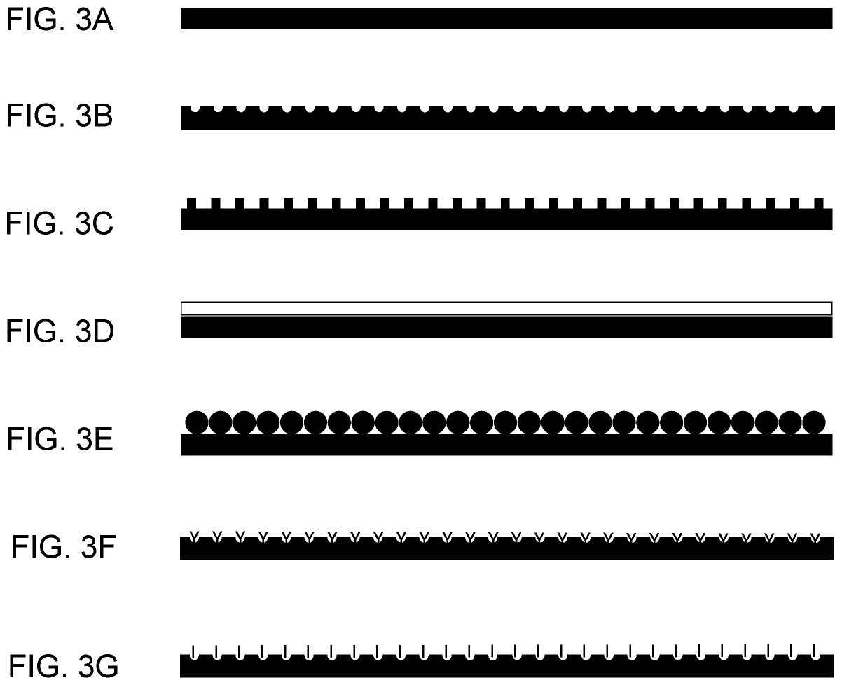

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 3A-3G illustrate different examples of cross-sectional surface profiles of a substrate. FIG. 3A illustrates a cross-sectional surface profile of a substrate having a completely planar surface. FIG. 3B illustrates a cross-sectional surface profile of a substrate having semi-spherical troughs or grooves. FIG. 3C illustrates a cross-sectional surface profile of a substrate having pillars, or alternatively or in conjunction, wells. FIG. 3D illustrates a cross-sectional surface profile of a substrate having a coating. FIG. 3E illustrates a cross-sectional surface profile of a substrate having spherical particles. FIG. 3F illustrates a cross-sectional surface profile of FIG. 3B, with a first type of binders seeded or associated with the respective grooves. FIG. 3G illustrates a cross-sectional surface profile of FIG. 3B, with a second type of binders seeded or associated with the respective grooves.

A binder may be configured to immobilize an analyte or reagent to an individually addressable location. In some cases, a surface chemistry of an individually addressable location may comprise one or more binders. In some cases, a plurality of individually addressable locations may be coated with binders. In some cases, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the total number of individually addressable locations, or of the surface area of the substrate, are coated with binders. The binders may be integral to the array. The binders may be added to the array. For instance, the binders may be added to the array as one or more coating layers on the array. The substrate may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the substrate may comprise an order of magnitude of at most about 10¹¹, 10¹⁰, 10⁹, 10⁸, 10⁷, 10⁶, 10⁵, 10⁴, 10³, 100, 10 or fewer binders.

The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis. The axis of rotation may or may not be an axis through the center of the substrate. In some instances, the systems, devices, and apparatus described herein may further comprise an automated or manual rotational unit configured to rotate the substrate. The rotational unit may comprise a motor and/or a rotor to rotate the substrate. For instance, the substrate may be affixed to a chuck (such as a vacuum chuck). The substrate may be rotated at a rotational speed of at least 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with a rotational velocity that is within a range defined by any two of the preceding values. The substrate may be configured to rotate with different rotational velocities during different operations described herein. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For controlled dispensing, the substrate may be rotating with a rotational frequency of no more than 60 rpm, no more than 50 rpm, no more than 40 rpm, no more than 30 rpm, no more than 25 rpm, no more than 20 rpm, no more than 15 rpm, no more than 14 rpm, no more than 13 rpm, no more than 12 rpm, no more than 11 rpm, no more than 10 rpm, no more than 9 rpm, no more than 8 rpm, no more than 7 rpm, no more than 6 rpm, no more than 5 rpm, no more than 4 rpm, no more than 3 rpm, no more than 2 rpm, or no more than 1 rpm. In some cases the rotational frequency may be within a range defined by any two of the preceding values. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, etc.).

In some cases, the substrate may be movable in any vector or direction. For example, such motion may be non-linear (e.g., in rotation about an axis), linear, or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. Alternatively or in addition, one or more conditions may be applied to attain a film thickness of at most 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm) or less. One or more conditions may be applied to attain a film thickness that is within a range defined by any two of the preceding values. The thickness of the film may be measured or monitored by a variety of techniques, such as thin film spectroscopy with a thin film spectrometer, such as a fiber spectrometer. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

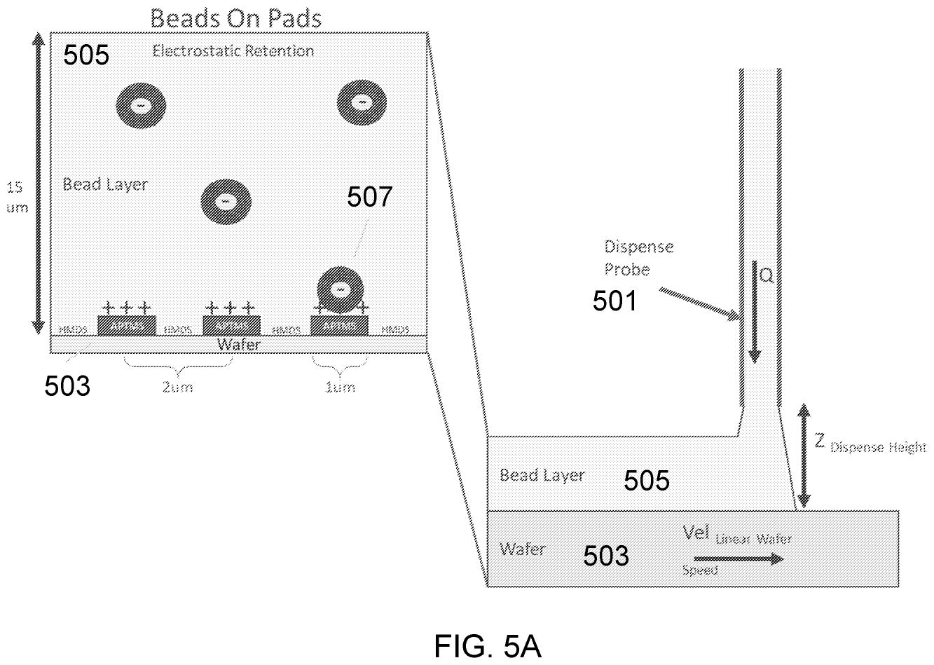

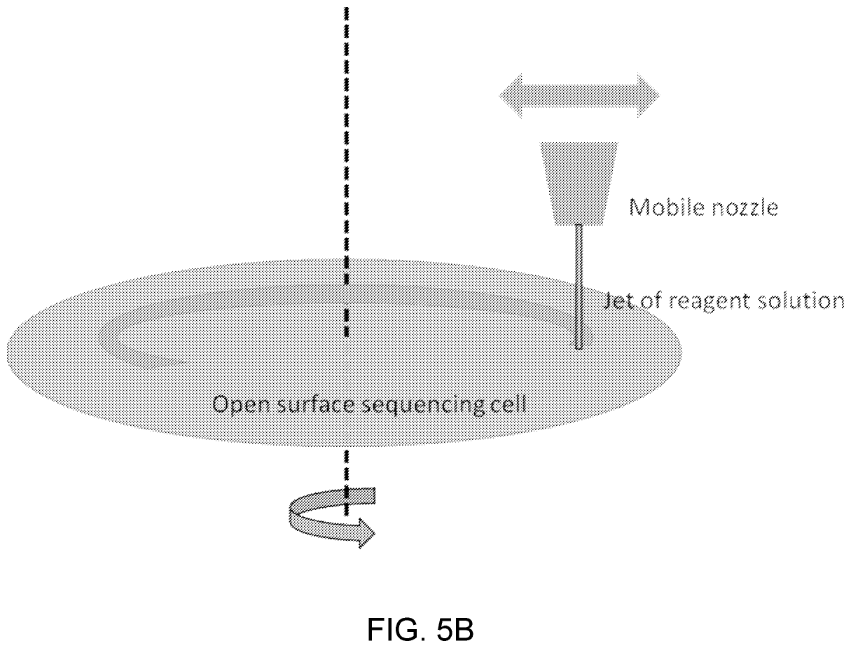

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. In some embodiments, two or more solutions or reagents are delivered to the substrate using the same or different delivery methods. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, the solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. In some embodiments, dispensing of each component of the two or more components may be temporally separated. Dispensing of each component may be performed using the same or different delivery methods. In some embodiments, direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, or the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

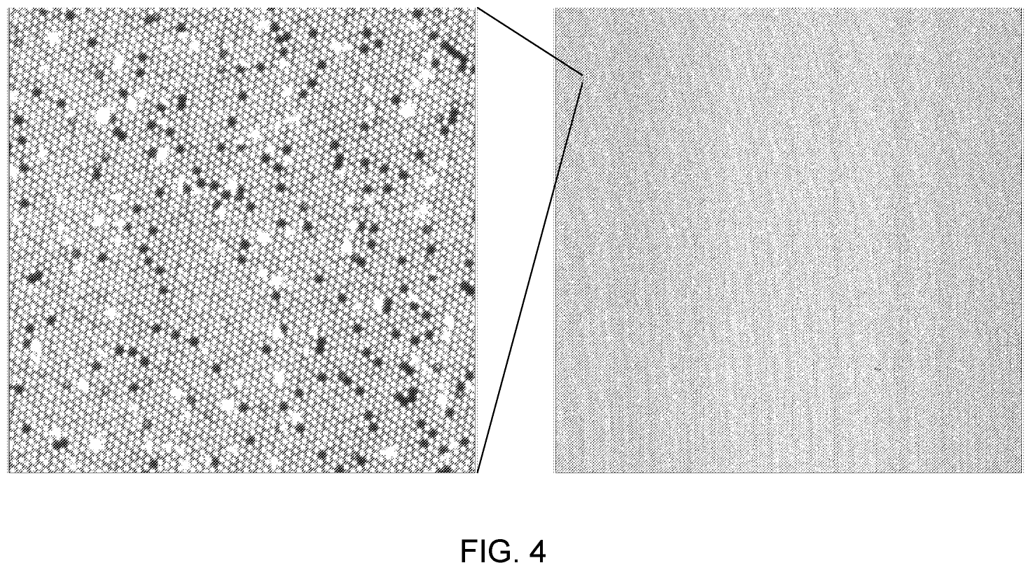

In some cases, a sample may be diluted such that the approximate occupancy of the individually addressable locations is controlled. In some cases, a sample may comprise beads, as described elsewhere herein, for example beads comprising nucleic acid colonies bound thereto. In some cases, an order of magnitude of at least about 10, 100, 1000, 10,000, 100,000, 1,000,000, 10,000,000, 100,000,000, 1,000,000,000, 10,000,000,000, 100,000,000,000 or more beads may be loaded on the substrate, such as to immobilize to as many individually addressable locations. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag that identifies a bead amongst a plurality of beads. FIG. 4 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. In some cases, after sample loading, a “bead occupancy” may generally refer to the number of individually addressable locations of a type comprising at least one bead out of the total number of individually addressable locations of the same type. A bead “landing efficiency” may generally refer to the number of beads that bind to the surface out of the total number of beads dispensed on the surface.

In some cases, beads may be dispensed to the substrate according to one or more systems and methods shown in FIGS. 5A-5B. As shown in FIG. 5A, a solution comprising beads may be dispensed from a dispense probe 501 (e.g., a nozzle) to a substrate 503 (e.g., a wafer) to form a layer 505. The dispense probe may be positioned at a height (“Z”) above the substrate. In the illustrated example, the beads are retained in the layer 505 by electrostatic retention, and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 505 a bead may successfully land on a first location of the first location type (as in 507). In the illustrated example, the location size is 1 micron, the pitch between the different locations of the same location type (e.g., first location type) is 2 microns, and the layer has a depth of 15 micron. FIG. 5B illustrates a reagent (e.g., beads) being dispensed along a path on an open surface of the substrate. As shown in FIG. 5B, a reagent solution may be dispensed from a dispense probe (e.g., a nozzle). The reagent may be dispensed on the surface in any desired pattern or path. This may be achieved by moving one or both of the substrate and the dispense nozzle. The substrate and the dispense probe may move in any configuration with respect to each other to achieve any pattern (e.g., linear pattern, substantially spiral pattern, etc.).

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A detectable signal, such as an optical signal (e.g., fluorescent signal), may be generated upon a reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by a detector (e.g., one or more sensors). For example, an optical signal may be detected via one or more optical detectors in an optical detection scheme described elsewhere herein. The signal may be detected during rotation of the substrate. The signal may be detected following termination of the rotation. The signal may be detected while the analyte is in fluid contact with a solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any optical source. In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise a controller. The controller may be operatively coupled to the one or more sensors. The controller may be programmed to process optical signals from each region of the rotating substrate. For instance, the controller may be programmed to process optical signals from each region with independent clocking during the rotational motion. The independent clocking may be based at least in part on a distance of each region from a projection of the axis and/or a tangential velocity of the rotational motion. The independent clocking may be based at least in part on the angular velocity of the rotational motion. While a single controller has been described, a plurality of controllers may be configured to, individually or collectively, perform the operations described herein.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate

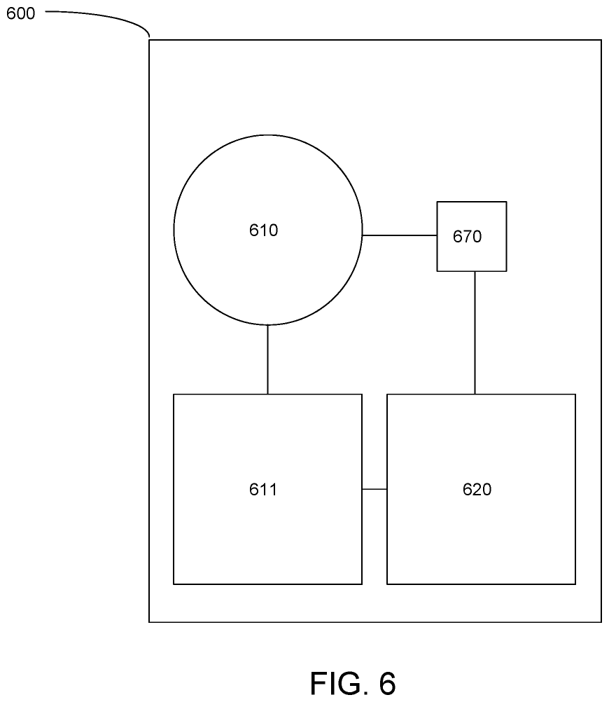

FIG. 6 shows a computerized system 600 for sequencing a nucleic acid molecule. The system may comprise a substrate 610, such as any substrate described herein. The system may further comprise a fluid flow unit 611. The fluid flow unit may comprise any element associated with fluid flow described herein. The fluid flow unit may be configured to direct a solution comprising a plurality of nucleotides described herein to an array of the substrate prior to or during rotation of the substrate. The fluid flow unit may be configured to direct a washing solution described herein to an array of the substrate prior to or during rotation of the substrate. In some instances, the fluid flow unit may comprise pumps, compressors, and/or actuators to direct fluid flow from a first location to a second location. The fluid flow unit may be configured to direct any solution to the substrate 610. The fluid flow system may be configured to collect any solution from the substrate 610. The system may further comprise a detector 670, such as any detector described herein. The detector may be in sensing communication with the substrate surface.

The system may further comprise one or more processors 620. The one or more processors may be individually or collectively programmed to implement any of the methods described herein. For instance, the one or more processors may be individually or collectively programmed to implement any or all operations of the methods of the present disclosure. In particular, the one or more processors may be individually or collectively programmed to: (i) direct the fluid flow unit to direct the solution comprising the plurality of nucleotides across the array during or prior to rotation of the substrate; (ii) subject the nucleic acid molecule to a primer extension reaction under conditions sufficient to incorporate at least one nucleotide from the plurality of nucleotides into a growing strand that is complementary to the nucleic acid molecule; and (iii) use the detector to detect a signal indicative of incorporation of the at least one nucleotide, thereby sequencing the nucleic acid molecule.

### High Throughput

An open substrate system of the present disclosure may comprise a barrier system configured to maintain a fluid barrier between a sample processing environment and an exterior environment. The barrier system is described in further detail in US20210354126A1, which is entirely incorporated herein by reference. A sample environment system may comprise a sample processing environment defined by a chamber and a lid plate, where the lid plate is not in contact with the chamber. The gap between the lid plate and the chamber may comprise the fluid barrier. The fluid barrier may comprise fluid (e.g., air) from the sample processing environment and/or the exterior environment and may have lower pressure than the sample environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Alternatively or in addition, for an operation, the systems (or any element thereof) may be maintained at a temperature of at most 100° C., 95° C., 90° C., 85° C., 80° C., 75° C., 70° C., 65° C., 60° C., 55° C., 50° C., 45° C., 40° C., 35° C., 30° C., 25° C., 20° C., or less. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation. In one example, a sample processing environment comprising a substrate as described elsewhere herein may be environmentally controlled from an exterior environment. The sample processing environment may be further divided into separate regions which are maintained at different local temperatures and/or relative humidities, such as a first region contacting or in proximity to a surface of the substrate, and a second region contacting or in proximity to a top portion of the sample processing environment (e.g., a lid). For example, the local environment of the first region may be maintained at a first set of temperatures and first set of humidities configured to prevent or minimize evaporation of one or more reagents on the surface of the substrate, and the local environment of the second region may be maintained at a second set of temperatures and second set of humidities configured to enhance or restrict condensation. The first set of temperatures may be the lowest temperatures within the sample processing environment and the second set temperatures may be the highest temperatures within the sample processing environment.

In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

In some instances, an open substrate is retained in the same or approximately the same physical location during processing of an analyte and subsequent detection of a signal associated with a processed analyte.

In some instances, different operations on or with the open substrate are performed in different stations. Different stations may be disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The barrier systems may be used to maintain different physical conditions of one or more different stations of the system, or portions thereof, as described elsewhere herein.

The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity.

In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

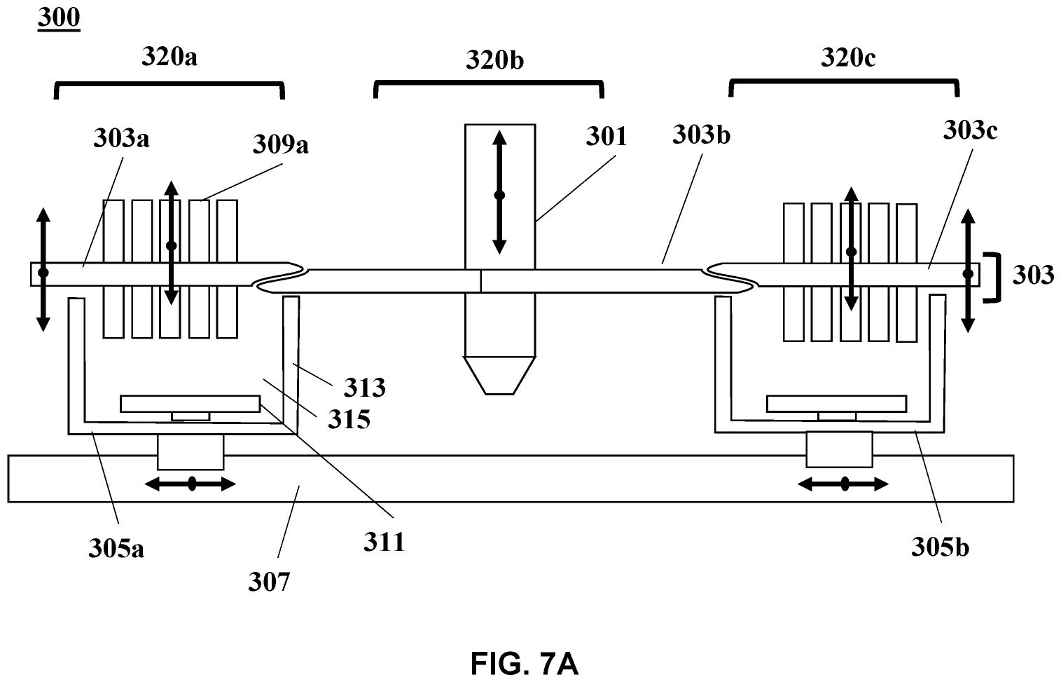

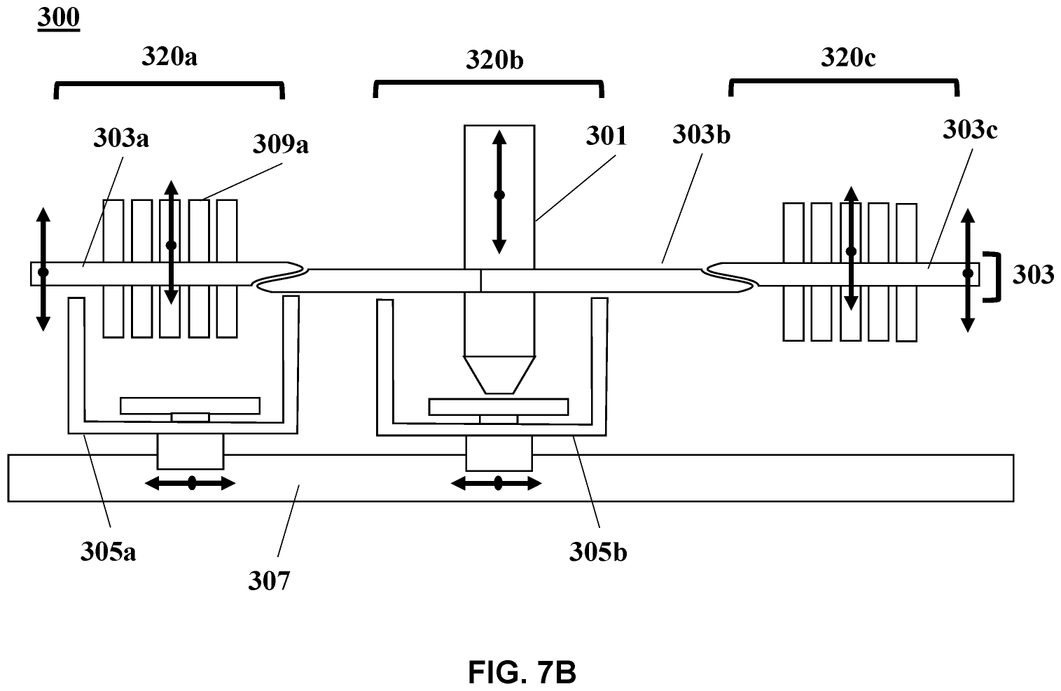

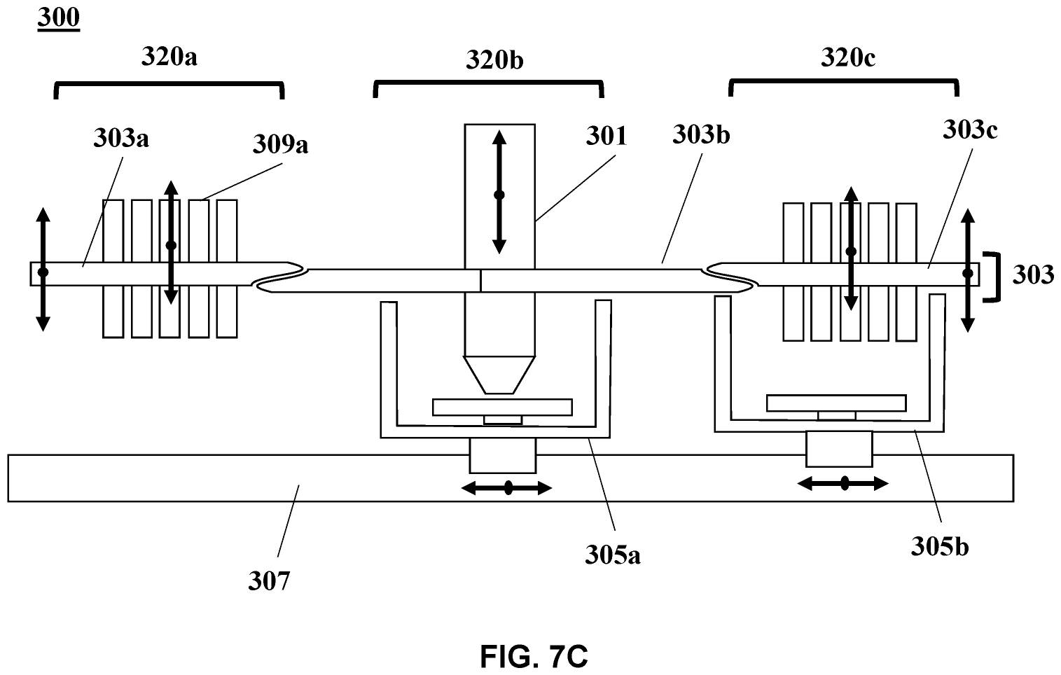

One or more modular sample environment systems (each having its own barrier system) can be used between the different stations. In some instances, the systems described herein may be scaled up to include two or more of a same station type. For example, a sequencing system may include multiple processing and/or detection stations. FIGS. 7A-7C illustrate a system 300 that multiplexes two modular sample environment systems in a three-station system. In FIG. 7B, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 7C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 7B, and this re-stationing can be repeated (e.g., between the configurations of FIGS. 7B and 7C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 7A.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

Provided herein are devices, systems, methods, compositions, and kits for spatial screening and spatial analysis of one or more analytes. Such devices, systems, methods, compositions, and kits can be applied in conjunction with, alternatively, or in addition to one or more operations in the sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein, also generally referred to herein as sequencing platforms.

### Spatial Screening

The systems, devices, and methods described herein may be useful for various spatial screening applications, such as to determine or identify information related to a spatial resolution within or of an analyte and/or sample.

Disclosed are methods and compositions to carry out omic-based studies, such as the characterization, measurement, and/or quantification of analytes of biological samples, with location information, using systems and methods described herein. In some cases, omic-based studies, such as the characterization, measurement, and/or quantification of analytes, may comprise genomics, epigenomics, lipidomics, proteomics, glycomics, transcriptomics, metabolomic, microbiomics, connectomics, or any derivatives thereof. In some cases, omic-based approach may study, characterize, measure, and/or quantify analytes or derivatives thereof of biological samples. An analyte may be or comprise any analyte described herein. In some instances, an analyte may comprise a nucleic acid, polypeptide, protein, carbohydrate, lipid, derivatives thereof, or any combinations thereof. A nucleic acid may be or comprise any nucleic acid molecule described herein. In some cases, a nucleic acid may comprise deoxyribonucleic acid (DNA), ribonucleic acid (RNA), locked nucleic acid (LNA), peptide nucleic acid (PNA), modified nucleic acid (XNA), derivative thereof, or any combination thereof. In some cases, a nucleic acid may comprise an endogenous or exogenous chemical modification. Such a modification may comprise a sugar modification, a base modification, a backbone modification, or an unnatural base pairing. For example, DNA may be methylated or acetylated. In some cases, a carbohydrate may comprise a monosaccharide, a polysaccharide, a lignin, derivatives thereof, or any combinations thereof. In some cases, a peptide or a protein may be modified. A modification of a protein or a peptide, in some cases, may comprise an ubiquitylation, sumolyation, ubiquitin-like modification, phosphorylation, methylation, acetylation, proteolysis, glycosylation, isoprenylation, deamidation, eliminylation, AMPlyation, ADP-ribosylation, redox, any derivatives thereof, or any combinations thereof.

A biological sample for spatial screening may be or comprise any biological sample described herein. In some cases, a biological sample may comprise a single cell, a group of cells, a tissue, an organ, or derivatives thereof. For example, a cell may comprise a blood cell or a tumor cell. In some cases, a biological sample may comprise a tissue or a portion thereof obtained by a biopsy. In some cases, a biological sample may be in a healthy state. In other cases, a biological sample may be in a disease state when compared to another biological in a healthy state. In some cases, a biological sample may comprise a tumor. In some instances, a biological sample may comprise multiple cells from a same organism. In other cases, a biological sample may comprise multiple cells from more than one organism. In one case, a biological sample may comprise an infected tissue. In instances, a biological sample may originate in in vitro culture. One such example may comprise an organoid.

In some cases, a biological sample may comprise a plurality of cells or tissue samples. A plurality of cells or tissue samples, in some cases, may comprise at least about 1×10² cells or tissue samples, at least about 1×10³ cells or tissue samples, at least about 1×10⁴ cells or tissue samples, at least about 1×10⁵ cells or tissue samples, at least about 1×10⁶ cells or tissue samples, at least about 1×10⁷ cells or tissue samples, at least about 1×10⁸ cells or tissue samples, at least about 1×10⁹ cells or tissue samples, at least about 1×10¹⁰ cells or tissue samples, at least about 1×10¹¹ cells or tissue samples, or at least about 1×10¹² cells or tissue samples. In other cases, a plurality of cells or tissue samples may comprise from about 5×10¹ to about 1×10² cells or tissue samples, from about 1×10² to about 5×10² cells or tissue samples, from about 5×10² to about 1×10³ cells or tissue samples, from about 1×10³ to about 5×10³ cells or tissue samples, from about 5×10³ to about 1×10⁴ cells or tissue samples, from about 1×10⁴ to about 5×10⁴ cells or tissue samples, from about 5×10⁴ to about 1×10⁵ cells or tissue samples, from about 1×10⁵ to about 5×10⁵ cells or tissue samples, from about 5×10⁵ to about 1×10⁶ cells or tissue samples, from about 1×10⁶ to about 5×10⁶ cells or tissue samples, from about 5×10⁶ to about 1×10⁷ cells or tissue samples, from about 1×10⁷ to about 5×10⁷ cells or tissue samples, from about 5×10⁷ to about 1×10⁸ cells or tissue samples, from about 1×10⁸ to about 5×10⁸ cells or tissue samples, from about 5×10⁸ to about 1×10⁹ cells or tissue samples, from about 1×10⁹ to about 5×10⁹ cells or tissue samples, from about 5×10⁹ to about 1×10¹⁰ cells or tissue samples, from about 1×10¹⁰ to about 5×10¹⁰ cells or tissue samples, from about 5×10¹⁰ to about 1×10¹¹ cells or tissue samples, from about 1×10¹¹ to about 5×10¹¹ cells or tissue samples, or from about 5×10¹¹ to about 1×10¹² cells or tissue samples.

In some instances, a cell or other sample may be deconstructed into individual analytes after each analyte is encoded with a location. Such deconstruction may allow the analytes to be processed by the systems and methods described herein. Such deconstruction may also facilitate or improve the processing by the systems and methods described herein. Once processed, the analytes can be reconstructed (e.g., spatially reconstructed) by detecting and digitally decoding the location using molecular labels encoding the respective locations of the analytes. In other cases, a biological sample may not undergo deconstruction to be processed by the systems and methods described herein.

The location of an analyte may comprise the spatial origin information of the analyte in a biological sample. The location of an analyte may comprise any location information of the analyte with respect to a biological sample. In some cases, a location may be a two-dimensional location. In other cases, a location may be a three-dimensional location. A location of an analyte may be encoded, such as via molecular encoding or digital encoding.

Encoding a location may comprise recording or specifying an addressable location of an analyte with a spatial reference system. A spatial reference system may comprise a Cartesian coordinate system (e.g., comprising 2 axes or 3 axes). A 2-axis coordinate system may specify an analyte on a two dimensional space. A 3-axis coordinate system may specify an analyte on a three dimensional space. In some cases, a spatial reference system may also comprise a pixel of an image. A spatial reference system may comprise any other coordinate system (e.g., polar coordinate system). A spatial reference system may comprise a reference point (e.g., origin, or non-origin point).

Molecular encoding of a location of an analyte may comprise linking a molecular label encoding a location to the analyte. Linking a molecular label to an analyte may comprise, for example, formation of a covalent or non-covalent bond, a binding between the molecular label and the analyte, a hybridization between the molecular label and the analyte, and/or generating a derivative of the analyte using the molecular label. A molecular label may comprise a barcode sequence.

In some cases, a molecular label encoding a location of an analyte may be the same molecular species as the analyte (e.g., a nucleic acid molecule encoding the location information of another nucleic acid molecule). In some cases, a molecular label encoding a location of an analyte may be the derivative of the analyte (e.g., a DNA molecule encoding the location information of a cDNA molecule reverse transcribed from an RNA molecule). In other cases, a molecular label encoding a location of an analyte may be a different molecular species from the analyte (e.g., a nucleic acid molecule encoding the location information of a peptide molecule). In some instances, a molecular label encoding a location of an analyte may comprise one molecule. In some cases, a molecular label encoding a location of an analyte may comprise more than one molecule. In some cases, a molecular label encoding a location of an analyte may comprise 2, 3, 4, 5, 6, 7, 8, 9, 10 or more molecules.

In some cases, a molecular label encoding a location of an analyte may encode the location of only the analyte. In other cases, a molecular label encoding a location of an analyte may also encode the location of another analyte. For example, the location of an analyte may be used to decode the location of its neighboring analyte or otherwise an additional analyte within proximity. In some cases, a molecular label encoding a location of an analyte may also encode the spatial information of analytes sharing the same molecular origin. For example, a molecular label of a nucleic acid of a cell can encode the location information of other nucleic acids from the same cell. In some instances, an analyte and a molecular label encoding a location information of the analyte may originate from the same sample. In some cases, an analyte and a molecular label encoding a location of the analyte may originate from two different samples. For example, the expression level of a marker nucleic acid in one biological sample may be used to identify a single cell based on the expression of the marker gene from the single cell sequencing result of another biological sample. In some cases, when an analyte and a molecular label encoding a location of the analyte originate from two different samples, the analysis of the location of the analyte may comprise use of an algorithm.

A molecular label encoding a location of an analyte may comprise a barcode sequence, a peptide sequence, a hybridization pattern, a fluorescence measurement, a fluorophore identification, an enzymatic reaction, derivatives thereof, or any combination thereof.

In some cases, encoding a location of an analyte may occur before the analyte is amplified. In some cases, encoding a location of an analyte may occur after the analyte is amplified. In some cases, encoding a location of an analyte may occur simultaneously when the analyte is amplified. In some cases, encoding a location of an analyte may occur without the analyte being amplified. In some cases, a molecular label encoding a location of an analyte may be amplified. In other cases, a molecular label encoding a location of an analyte may not be amplified. In some cases, an amplification reaction or process may comprise any amplification reaction or process described herein. In some instances, an analyte or a molecular label encoding a location of the analyte may also be extended, ligated, cleaved, circularized, linearized, or activated. In some instances, a photoactivation may be required to extend, ligate, cleave, or activate an analyte or a molecular label encoding a location of the analyte.

In some instances, the molecular label encoding a location of an analyte may associate with the analyte. In other cases, the molecular label may associate with a derivative of the analyte. In some instances, the molecular label encoding a location of an analyte may not associate with the analyte or a derivative of the analyte.

In some instances, the molecular label encoding a location of an analyte may link to one region of the analyte. In other cases, the molecular label encoding a location of an analyte may link to more than one region of the analyte. For example, a nucleic acid sequence encoding a location of an analyte may be designed to bind to multiple regions of another nucleic acid molecule.

In some instances, an analyte and a molecular label encoding a location of the analyte may be processed or analyzed in the same reaction. For example, a first nucleic acid molecule and a second nucleic molecule encoding a location of the first nucleic acid molecule may be processed by a sequencing reaction. In other cases, an analyte and a molecular label encoding a location of the analyte may be processed or analyzed in different reactions. For example, a first nucleic acid molecule may be processed by a sequencing reaction while a second nucleic acid molecule encoding a location of the first nucleic acid molecule may be processed by fluorescence hybridization and imaging.

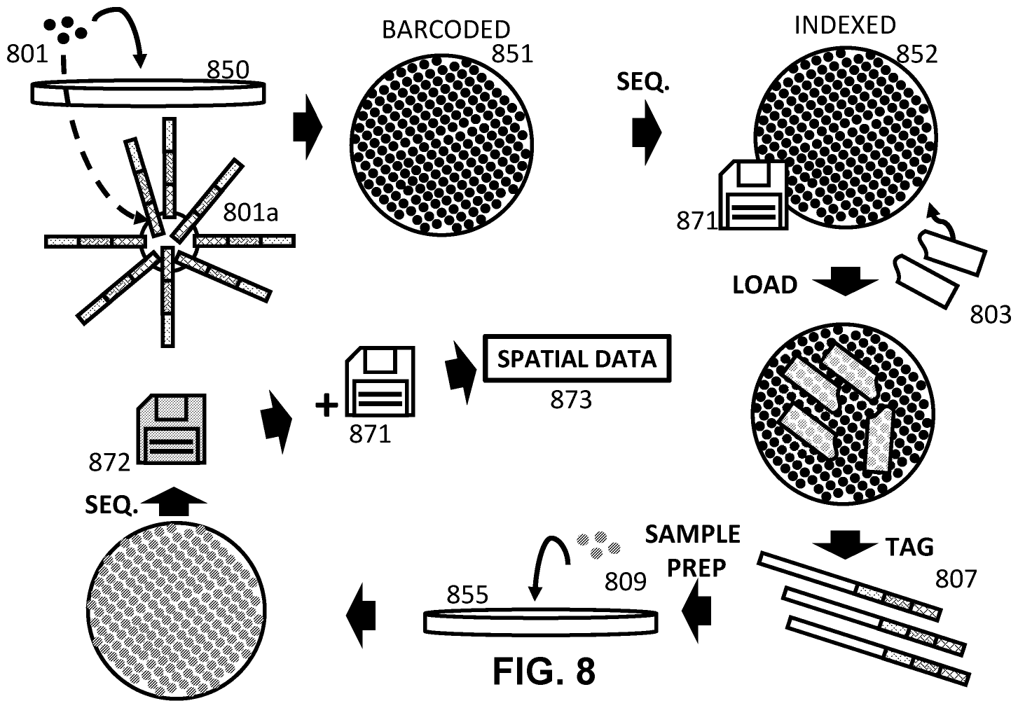

FIG. 8 illustrates an example method for spatially analyzing a biological sample using molecular labels with substrate-based sample processing systems. The method may comprise (i) immobilizing a plurality of beads 801 to individually addressable locations of a substrate 850 to provide a barcoded substrate 851. Each bead of the plurality of beads may comprise oligonucleotide molecules coupled thereto (e.g., 801a). The oligonucleotide molecules coupled to the beads may comprise barcode sequences, for example such that each bead comprises a different barcode sequence amongst the plurality of beads. In some cases, each bead may comprise a colony of oligonucleotide molecules comprising an identical barcode sequence that is unique to said bead amongst the plurality of beads. The oligonucleotide molecules may comprise additional functional sequences, such as a primer sequence, adapter sequence, additional barcode sequence, and/or capture sequence. The oligonucleotide molecules may function as molecular labels. The method may then comprise (ii) sequencing the oligonucleotide molecules on the barcoded substrate 851 using one or more operations of the sequencing workflow 100 (e.g., sequencing 107 and data analysis 108) and the sample processing systems described herein, to generate indexing data 871 which catalogs or indexes the individually addressable locations of the substrate with the barcode sequences of the oligonucleotide molecules, to provide an indexed, barcoded substrate 852. That is, the identity of a particular barcode sequence may be indexed to a particular individually addressable location. The method may comprise (iii) loading a biological sample 803 to the indexed, barcoded substrate 852 to tag analytes in the biological sample with the oligonucleotide molecules in a spatially dependent manner, to provide spatially tagged analytes 807 that comprise the barcode sequences or derivatives thereof. That is, analytes within the biological sample that are located at a particular individually addressable location (as loaded on the indexed, barcoded substrate 852) may be contacted with the oligonucleotide molecules located and indexed at the particular individually addressable location. The method may comprise (iv) processing the spatially tagged analytes using one or more operations of the sequencing workflow 100 (e.g., any or all of operations 101-108) and the sample processing systems described herein, to generate template nucleic acids immobilized to a second plurality of beads 809, the template nucleic acids comprising (1) sequences associated with the analytes (e.g., sequence of the analyte itself, sequence associated with a probe used to capture the analyte, etc.) and (2) sequences associated with the barcode sequences, where the second plurality of beads 809 is immobilized to individually addressable locations of a second substrate 855. The method may comprise (v) sequencing the template nucleic acids on the second substrate 855 using one or more operations of the sequencing workflow 100 (e.g., sequencing 107 and data analysis 108) and the sample processing systems described herein, to generate sequencing data 872 for the template nucleic acids. The method may comprise (vi) using the sequencing data 872 to spatially resolve the analytes based at least in part on identifying the barcode sequences and their indexed locations on the substrate using the indexed data 871, to generate spatial data 873 of the analytes and/or the biological sample.

After the biological sample 803 is loaded to the indexed, barcoded substrate 852, the biological sample may be incubated with the reagents to permit contact between the oligonucleotide molecules with the analytes, or derivatives thereof, thereby tagging the analytes, or derivatives thereof. Such contact may occur via the oligonucleotide molecules traveling to the analytes or derivatives thereof, after their release from the beads 801; via the analytes or derivatives thereof traveling to the oligonucleotide molecules pre-release or post-release of the oligonucleotide molecules from the beads; and/or via both. The sample may be incubated with the reagents (e.g., oligonucleotide molecules, release-facilitating reagents such as enzymes, UV light, etc.) for any period of time. For example, the sample may be incubated for at least about 1 minute (min), 2 min, 3 min, 4 min, 5 min, 6 min, 7 min, 8 min, 9 min, 10 min, 20 min, 30 min, 40 min, 50 min, 60 min, 90 min, 120 min, 150 min, 180 min, 4 hours (h), 5 h, 6 h, 7 h, 8 h, 9 h, 10 h, 11 h, 12 h or more. Alternatively or in addition, the sample may be incubated at most about 12 h, 11 h, 10 h, 9 h, 8 h, 7 h, 6 h, 5 h, 4 h, 180 min, 150 min, 120 min, 90 min, 60 min, 50 min, 40 min, 30 min 20 min, 10 min, 9 min, 8 min, 7 min, 6 min, 5 min, 4 min, 3 min, 2 min, 1 min or less. Incubation may be performed at various reaction conditions (e.g., temperature, pH, salt concentration, etc.). The spatially tagged analytes may be processed or repaired prior to, during, or subsequent to isolation, for example to perform one or more of the following operations: cleaving at a cleavage site such as to remove a blocking group, ligating, denaturing, performing extension reactions, and other operations.

The oligonucleotide molecules may be single-stranded. The oligonucleotide molecules may be double-stranded. The oligonucleotide molecules may be partially double-stranded. A bead (e.g., 801) may comprise any number of oligonucleotide molecules attached thereto, for example on the order of 10, 10², 10³, 10⁴, 10⁵, or more.

A barcode sequence on an oligonucleotide molecule may comprise at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more bases. Alternatively or in addition, the barcode sequence may comprise at most about 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or fewer bases. Any functional sequence on an oligonucleotide molecule may comprise at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more bases. Alternatively or in addition, any functional sequence may comprise at most about 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or fewer bases. The barcode sequence may be unique and common to a bead amongst a plurality of beads. In some cases, the barcode sequence may be substantially unique to a bead amongst a plurality of beads such that at least about 50%, 55%, 60%, 65%, 70%, 75%, 80%, 85%, 90%, 91%, 92%, 93%, 94%, 95%, 96%, 97%, 98%, 99% or more of beads in the plurality of beads comprise unique barcode sequences not contained by any other bead in the plurality of beads. The methods, systems, compositions, and kits of the present disclosure may comprise a set of beads comprising any number of beads. The methods, systems, compositions, and kits of the present disclosure may comprise at least two sets of beads comprising any number of beads. In some cases, there may be a number of beads within a set of beads at least on the order of 10², 10³, 10⁴, 10⁵, 10⁶, 10⁷, 10⁸, 10⁹, 10¹⁰, 10¹¹, 10¹², 10¹³, 10¹⁴, or more beads. In some cases, there may be at least a number of beads within a set of beads to correspond to a number of individually addressable locations available on the substrate to which the beads are loaded.

The two substrates referred to in the methods described with respect to FIG. 8 may be identical in shape, size, material, and/or form. The two substrates may be processable (e.g., analytes thereon sequence-able) by the same sequencing platform, such as the sample processing systems described herein.

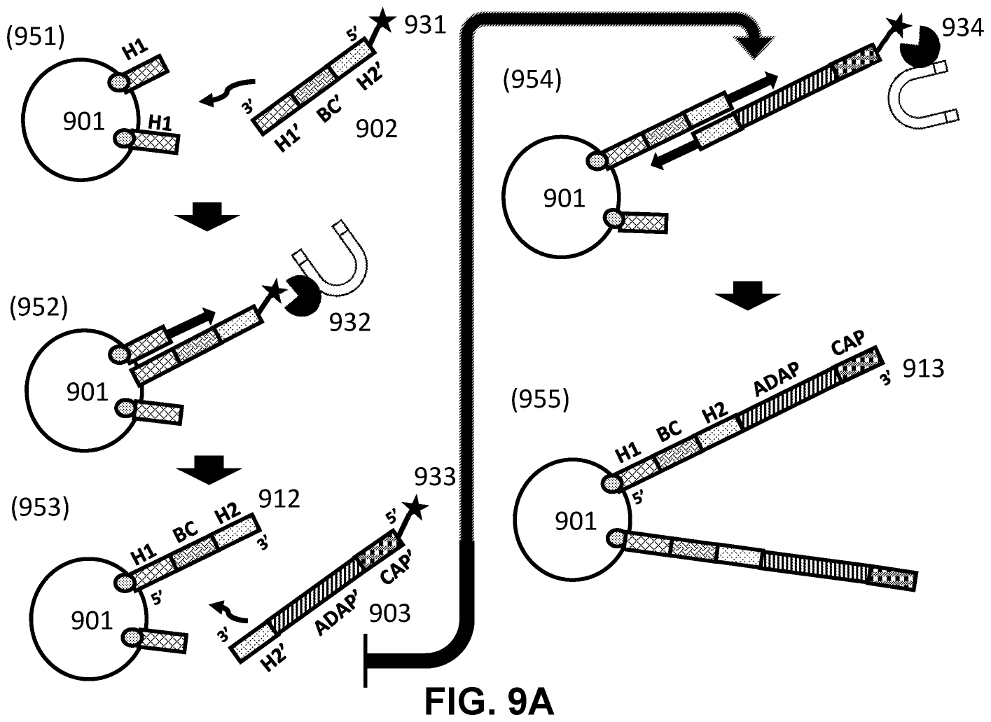

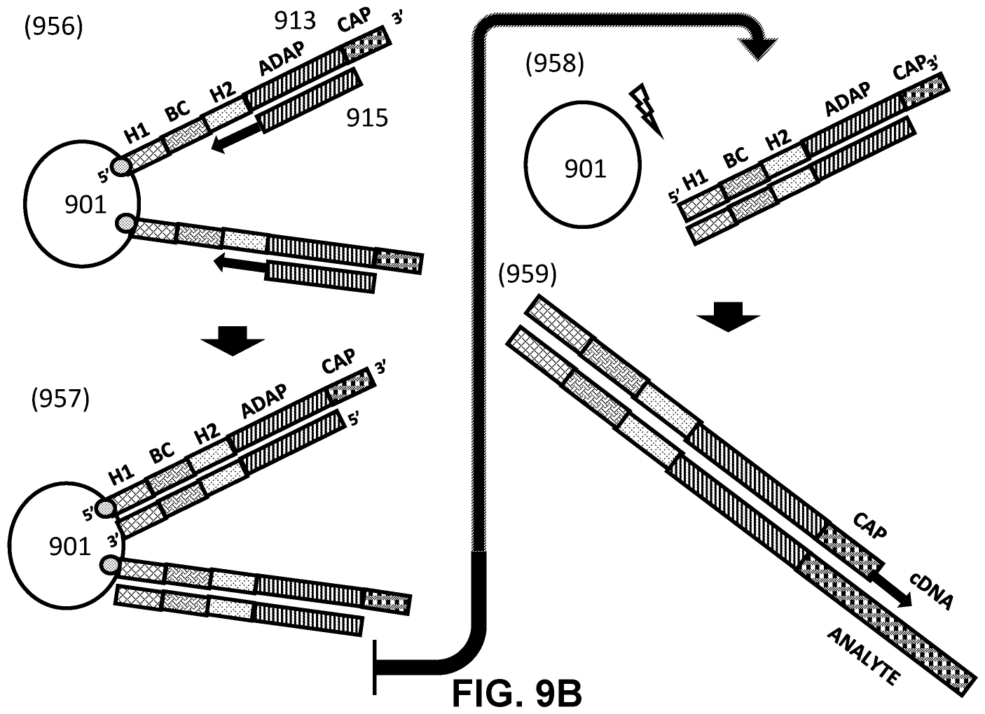

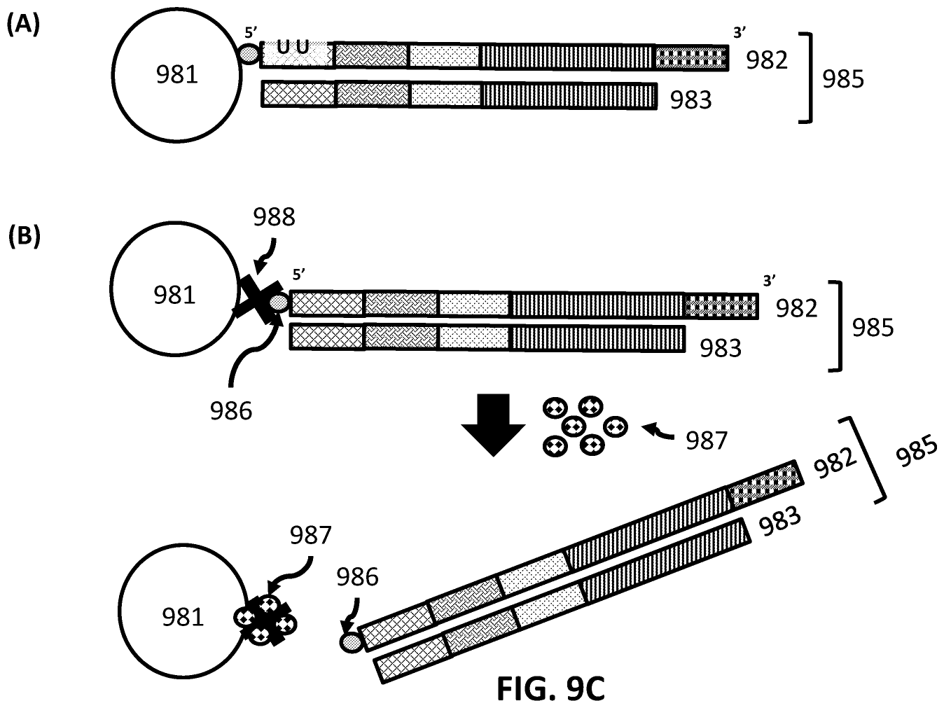

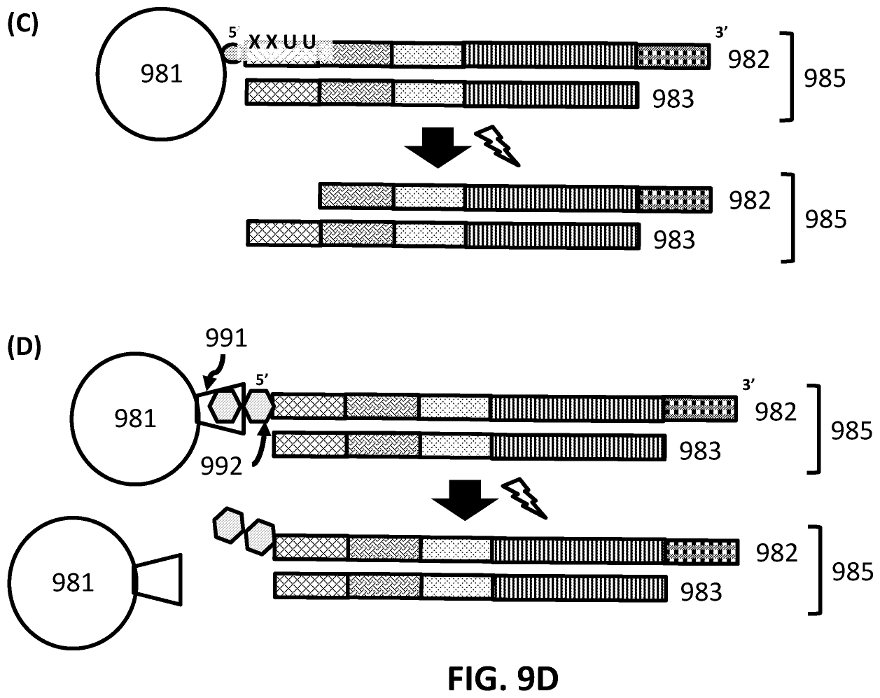

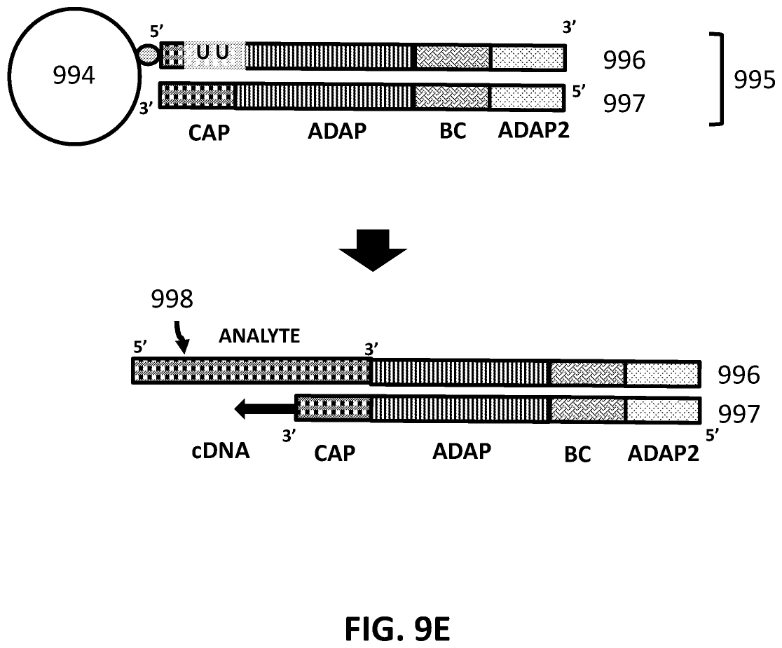

FIGS. 9A-B illustrate an example method for generating and using a bead comprising molecular labels. The bead comprising molecular labels may be used, for example, in the methods described with respect to FIG. 8 as the plurality of beads 801.

In (951), a bead 901 comprising a plurality of oligonucleotide molecules may be contacted with a barcode library molecule 902. The plurality of oligonucleotide molecules may each comprise a common first handle (H1). In some cases, the plurality of oligonucleotide molecules (and/or complementary strands hybridized thereto) may be releasable from the bead 901, such as via a cleavage site (e.g., abasic site). Various releasing and/or cleaving mechanisms between oligonucleotide molecules (or strands thereof) and beads are described elsewhere herein. A barcode library molecule 902 may comprise, from 3′ to 5′, a complement of the first handle (H1′), a complement of a bead barcode sequence (BC′), and a complement of a second handle (H2′) sequence. The complement of the bead barcode sequence (BC′) may vary across different barcode library molecules that are contacted to different beads, such that each bead is contacted with a unique complement of the bead barcode sequence.

In (952), an oligonucleotide molecule of the bead 901 may hybridize to the barcode library molecule 902 and be extended to generate a barcode molecule 912 coupled to the bead 901 as shown in (953), the barcode molecule 912 comprising, from 5′ to 3′, the first handle sequence (H1), a bead barcode sequence (BC), and the second handle sequence (H2). In some cases, the barcode library molecule 902 may comprise a capture moiety 931 (e.g., biotin) which may be captured by a capturing group 932 (e.g., streptavidin coupled to a magnet) at some time after hybridization between the oligonucleotide molecule and the barcode library molecule. A subset of beads which have hybridized to barcode library molecules (e.g., 902) each comprising the capture moiety (e.g., 931) may be isolated from, and thus enriched from, a larger population of beads. Various capture mechanisms that can be used for enrichment are described elsewhere herein. In some cases, when a population of beads is processed simultaneously, the population of beads may be provided with a predetermined relative concentration of barcode library molecules such that each bead is hybridized to at most one barcode library molecule, or only a small fraction of beads is hybridized to more than one barcode library molecule. For example, the beads may be provided at a much higher concentration than the barcode library molecules (e.g., 2:1, 3:1, 4:1, 5:1, 6:1, 7:1, 8:1, 9:1, 10:1, or higher beads: barcode library molecules). Beneficially, this may prevent polyclonality where a single bead contains two or more bead barcode sequences (BCs).

In (953), the bead 901 comprising the barcode molecule 912 may be subjected to amplification, such as ePCR, with a capture barcode library molecule 903 to generate a plurality of capture barcode molecules (e.g., 913) coupled to the bead. The capture barcode library molecule 903 may comprise, from 3′ to 5′, the complement of the second handle (H2′) sequence, a complement of an adapter (ADAP′) sequence, and a complement of a capture (CAP′) sequence. In some cases, ePCR amplification may comprise partitioning a plurality of beads (e.g., beads comprising the barcode molecules, e.g., 912) with a plurality of capture barcode library molecules (e.g., 903), and performing extension and/or amplification reactions within the partitions (e.g., droplets). That is, operations (951)-(952) (contacting of beads with barcode library molecules to generate beads comprising barcode molecules) may occur outside partitions, such as in bulk solution reaction environments, while operations (953)-(954) (contacting of beads comprising barcode molecules with capture barcode library molecules to generate capture barcode molecules) occur in partitions (e.g., droplets). In some cases, ePCR amplification may comprise partitioning a plurality of beads (e.g., beads comprising the oligonucleotide molecules) with a plurality of barcode library molecules (e.g., 902) and a plurality of capture barcode library molecules (e.g., 903), and performing extension and/or amplification reactions within the partitions (e.g., droplets). That is, in some cases, operations (951)-(954) (contacting of beads with barcode library molecules and capture barcode library molecules to generate beads comprising capture barcode molecules) may occur within partitions.

During amplification, in (954), a barcode molecule (e.g., 912) of the bead 901 may be hybridized to the capture barcode library molecule 903 and extended to generate a capture barcode molecule 913 coupled to the bead 901 as shown in (955), the capture barcode molecule 913 comprising, from 5′ to 3′, the first handle sequence (H1), the bead barcode sequence (BC), the second handle sequence (H2), the adapter sequence (ADAP), and a capture sequence (CAP). In some cases, when a population of beads is processed simultaneously, the population of beads may be provided with a predetermined relative concentration of capture barcode library molecules such that each bead is hybridized to at most one capture barcode library molecule, or only a small fraction of beads is hybridized to more than one capture barcode library molecule. For example, the beads may be provided at a much higher concentration than the capture barcode library molecules (e.g., 2:1, 3:1, 4:1, 5:1, 6:1, 7:1, 8:1, 9:1, 10:1, or higher beads: capture barcode library molecules). In some cases, the capture barcode library molecule 903 may comprise a capture moiety 933 (e.g., biotin) which may be captured by a capturing group 934 (e.g., streptavidin coupled to a magnet). A subset of beads which have hybridized to capture barcode library molecules (e.g., 903) each comprising the capture moiety (e.g., 933) may be isolated from, and thus enriched from, a larger population of beads. Various capture mechanisms that can be used for enrichment are described elsewhere herein. After amplification, in (955), the bead 901 may comprise double-stranded oligonucleotide molecules (e.g., comprising a first strand comprising a copy of the capture barcode molecule 913 and a second strand comprising its reverse complement), which may be denatured or otherwise processed such that the bead comprises single-stranded oligonucleotide molecules that are copies of the capture barcode molecule 913.

The capture sequence may be configured to capture an analyte, such as an RNA molecule. For example, the capture sequence may comprise a polyT sequence configured to capture a polyA tail sequence of an mRNA molecule. In another example, the capture sequence may comprise a random sequence, a targeted sequence, or any other sequence designed to capture an analyte sequence, or derivative thereof. In some cases, the capture sequence may comprise a random n-mer sequence. In some examples, for targeted mRNA assays, the capture sequence may comprise a target mRNA sequence (or derivative thereof). In some examples, for targeted genomic DNA (gDNA) assays, the capture sequence may comprise a target gDNA sequence (or derivative thereof). In some examples, for antibody assays, the capture sequence may comprise a sequence configured to capture an oligonucleotide conjugated to one or more antibodies (e.g., DNA capture tags), or a derivative thereof. In some examples, for assays that utilize template switching reactions, the capture sequence may comprise a sequence configured to capture a product of a reverse transcription reaction, such as a polyG sequence. In some examples, for assays that utilize one or more probes, the capture sequence may comprise a sequence corresponding to a sequence of the probe, to a molecule associated with the probe, or derivative thereof. In some examples, the capture sequence may be part of a single strand portion, a double strand portion, or partially double-stranded complex. In some examples, the capture sequence may be part of a hybrid DNA/RNA complex. In some examples, for a transposition assay concerning gDNA analytes, after a transposition reaction (e.g., subsequent to Tn5 transposase treatment of gDNA, where the Tn5 transposase comprises one or more barcode and/or adapter sequences), a partially double-stranded analyte may be generated. In some examples, at least one end of the partially double-stranded analyte may comprise an overhang comprising a barcode and/or adapter sequence. A capture sequence may be configured to capture the overhang of the partially double-stranded analyte comprising the barcode and/or adapter sequence. One or more gap filling and/or ligation reactions may be performed to join the partially double-stranded bridge construct and transposition analyte.

It will be appreciated that the barcode library molecule 902 and/or the capture barcode library molecule 903 may be designed to include any functional sequence in a predetermined order within the capture barcode molecules in the amplified bead. For example, the capture barcode molecules may comprise a primer sequence, adapter sequence, additional barcode sequence (e.g., sample barcode sequence), unique molecular identifier (UMI) sequence (e.g., which is unique to a single molecule amongst multiple molecules coupled to a bead), index sequence, and/or capture sequence in any desired order. For example, FIG. 9E shows an example of a bead with a different barcode design, which is described elsewhere herein.

The amplified beads comprising the capture barcode molecules (e.g., 913) may be used in accordance with the methods and systems described herein, for example, as the plurality of beads 801 in the methods described with respect to FIG. 8. In one example, as shown in FIGS. 9A-9B, the capture barcode molecules may comprise a capture sequence at a 3′ end and a sequencing primer sequence that is 5′ to the capture sequence. For example, the sequencing primer sequence may be or be a part of the adapter sequence (ADAP). According to the methods described in FIG. 8, during sequencing and indexing of the barcoded substrate in which the barcode sequences are identified, as shown in (956), a sequencing primer 915 may be hybridized to the sequencing primer sequence (e.g., ‘ADAP’) and thus initiate sequencing of the barcode library molecule without having to read through the capture sequence. After sequencing, as shown in (957), at least a portion of the capture barcode molecules (e.g., 913) may be hybridized to a complementary strand which are the extension products of sequencing primers (e.g., 915). In (958), one or both strands of the oligonucleotide molecules coupled to the bead may be released. For example, the oligonucleotide molecules may be released, and the released molecules may capture analytes of a biological sample. In another example, the oligonucleotide molecules may be released post-capture of analytes onto the bead. In one example, as shown in (959), an analyte molecule hybridizes to the capture sequence (CAP) of the oligonucleotide molecule, and the first strand (comprising the capture sequence) is extended using the analyte molecule as a template to generate cDNA.

In another example, as shown in FIG. 9E, a capture barcode molecule 995 of bead 994 may comprise a capture sequence (CAP) proximal to the bead. The capture barcode molecule 995 may comprise a first strand 996 that is coupled to the bead 994, and comprising from 5′ to 3′, a complement of a capture sequence (CAP′), a complement of an adapter sequence (ADAP′), a complement of a barcode sequence (BC′), and a complement of a sequencing primer sequence (ADAP2′). The second strand 997 may comprise a reverse complement of the first strand 996, comprising from 3′ to 5′, the capture sequence (CAP), the adapter sequence (ADAP), the barcode sequence (BC), and the sequencing primer sequence (ADAP2). In some cases, the first strand 996 may comprise a cleavage site, denoted ‘U,’ at or adjacent to the 5′ end. The oligonucleotide molecule 995 may be released from the bead 994 by cleaving at the cleavage site. The released molecule may hybridize with an analyte sequence 998, and the second strand 997 may be extended using the analyte as a template to generate cDNA.

In some cases, after generating cDNA (e.g., FIG. 9B, 9E) and/or after an analyte sequence has been barcoded, a template switching reaction may be performed. The cDNA transcript may comprise an additional sequence (e.g., polyC sequence) at the end of the reverse transcription reaction. For example, the additional sequence may comprise bases that are added as a result of terminal transferase activity. A template switching oligonucleotide may be provided, the template switching oligonucleotide comprising a switch sequence (e.g., polyG sequence, poly rG sequence, etc.) at a 3′ end that is configured to bind to the additional sequence (e.g., polyC sequence). The template switching oligonucleotide may comprise one or more functional sequences (e.g., barcode sequence, adapter sequence, index sequence, etc.) at the 5′ end. After binding the template switching oligonucleotide to the cDNA transcript, the template switching oligonucleotide may be extended. In another example, a strand of a barcoded analyte, or derivative thereof, can be contacted with a single Tn5 adapter, reverse transcription performed, and PCR performed. In another example, a strand of a barcoded analyte, or derivative thereof, may be contacted with an enzyme to shear the nucleic acid molecule, a splint adapter may be ligated, and PCR performed. One or more downstream processes may comprise multiple rounds of PCR. One or more downstream processes may comprise enzymatic fragmentation. One or more downstream processes may comprise end repair of the A-tail.

An oligonucleotide molecule may be releasably coupled to the bead to allow for future cleavage or release from the bead. The oligonucleotide molecules coupled to the beads, as described herein, may exist in single-stranded or double-stranded form. Various release or cleavage mechanisms may be used. For example, one or more stimuli may be applied, including light stimuli, heat stimuli, chemical stimuli, enzymatic stimuli, magnetic stimuli, electrical stimuli, and other stimuli, or combination thereof to cleave or effect cleavage of the oligonucleotide molecule from the bead, or if the oligonucleotide molecule is at least partially double-stranded molecule, cleave or effect cleavage of one or both strands from the bead.

For example, where the oligonucleotide molecule is a double-stranded molecule, a strand displacing polymerase (e.g., a polymerase with relatively strong ability to strand displacement compared to polymerases that lack strand displacement activity such as T4 and T7 DNA polymerases) and a primer may be provided under conditions sufficient to displace one of the strands from the oligonucleotide molecule (e.g., displace the strand comprising the capture sequence). Examples of strand displacing polymerases include, but are not limited to, Bst DNA polymerase, large fragment polymerase, and Φ29 polymerase. In some cases, for strand displacement in the 5′ to 3′ direction, the strand displacing polymerase may generate or leverage a flap at the 5′ end. Where strand displacement is in the 3′ to 5′ direction, the strand displacing polymerase may generate or leverage a flap at the 3′ end. In another example release mechanism, an enzyme may be provided to digest at least a portion of a strand to release a remaining portion of that strand or the other strand. The enzyme may comprise 5′ to 3′ exonuclease activity. Examples of enzymes with 5′ to 3′ exonuclease activity include, but are not limited to, Lambda Exonuclease, T7 Exonuclease, T5 Exonuclease, Exonuclease V, and Exonuclease VIII. The enzyme may comprise 3′ to 5′ exonuclease activity. Examples of enzymes with 3′ to 5′ exonuclease activity include, but are not limited to, Exonuclease III and Exonuclease V. In one example, a 5′ end of the second strand may be phosphorylated and subjected to Lambada Exonuclease.

In some cases, an oligonucleotide molecule may be immobilized to the bead via a cross-link or other linker. The cross-link or other linker may be cleaved to release the oligonucleotide molecules, or portion thereof, such as by applying one or more stimuli, including light stimuli, heat stimuli, chemical stimuli, magnetic stimuli, electrical stimuli, and other stimuli, or combination thereof. In some cases, the oligonucleotide molecules may be immobilized to the bead via a photo-cross-link. A photo-cross-link may be generated by a photo cross-linking reaction. In some instances, an oligodeoxynucleotide (ODN) comprising 3-cyanovinylcarbazole nucleoside (^{CNV}K) can be subjected to photoirradiation conditions to photo-cross-link a target pyrimidine and the ^{CNV}K. In some instances, irradiation is provided at 366 nm for about 1 second for photo-cross-linking to thymine, and for up to about 25 seconds for photo-cross-linking to cytosine. In this example, irradiation provided at 312 nm for about 3 minutes can reverse the cross-link. Various other cross-linking reagents may be used to generate a cross-link (e.g., chemical cross-link). In some cases, heat may be applied to denature a double-stranded molecule to facilitate release of at least one of the strands from the bead. The heat stimulus can be combined with cross-linking reactions. For example, a strand may be released from the bead by applying a heat stimulus. In some cases, additionally, a primer may be cross-linked (e.g., photo-cross-linked) to the oligonucleotide molecule and extended, such as to prevent re-hybridization of the released strand to the remaining strand.

In some cases, a strand of the oligonucleotide molecule may comprise one or more features (e.g., a blocking group) that prevent digestive activity by an enzyme on that strand. In some cases, a strand of the oligonucleotide molecule may comprise a cleavage site. In some cases, the oligonucleotide molecule may be capped by an uracil, a ribonucleotide, or other modified nucleotide to facilitate digestion of the second strand to release the first strand. In some cases, the oligonucleotide molecule may comprise one or more nicks (or nicks may be created) to facilitate strand displacement and/or digestive activity by an enzyme. In some cases, the oligonucleotide molecule may comprise one or more cleavable or digestible moieties (e.g., ribonucleotides, uracil, etc.) for cleavage or digestion by one or more enzymes (e.g., RNase HII). In some examples, a USER cleavage reaction may be performed to process the cleavable or digestible moieties from the double stranded molecules and release the remaining molecule from the bead. In the USER cleavage reaction, a USER (uracil-specific excision reagent) enzyme may generate a nucleotide gap at a location of a uracil base (e.g., dU) in the molecule and facilitate cleavage.

FIGS. 9C-9D illustrates various bead release mechanisms. For all panels, shown is a bead 981 comprising a double-stranded oligonucleotide molecule 985, which comprises a first strand 982 and a second strand 983.

In panel (A), the first strand 982 may comprise a cleavage site, denoted “U”, at or adjacent to the 5′ terminus. The cleavage site may be cleaved to release the first strand from the bead. One or both strands may have one or more cleavage sites. In some cases, a USER enzyme mix may be used for the cleavage reaction. The USER enzyme mix may comprise uracil DNA glycosylase (UDG), which removes the sugar and creates an abasic site (AP site), and endonuclease (e.g., endonuclease VIII), which binds to the AP Site and cleaves. In some cases, to accelerate cleavage, the components of the USER enzyme mix may be divided and provided independently, and later combined. For example, with reference to the methods of FIG. 8, the UDG may be provided separately to the indexed, barcoded substrate 852 comprising the beads immobilized thereto, prior to loading of the sample (e.g., tissue), and the endonuclease may be provided separately to the sample (e.g., tissue). The cleavage reaction may take effect after the sample is loaded onto the indexed, barcoded substrate. Beneficially, the AP sites may be created as a substrate-priming step, prior to loading of the sample. In some cases, the endonuclease may be replaced with an APE1 enzyme in the USER enzyme mix, which cleaves multiple times and is Mg²⁺-dependent (as opposed to endonuclease VIII which is Mg²⁺-independent). In an example, with reference to the methods of FIG. 8, the UDG and APE1 enzyme may be provided to the indexed, barcoded substrate 852 comprising the beads immobilized thereto, without Mg²⁺, and prior to loading of the sample, to prime the substrate and form the AP sites. The APE1 enzyme may bind to the AP sites, without cleavage activity due to the lack of the Mg²⁺. Separately, Mg²⁺ may be provided separately to the sample (e.g., tissue). The cleavage reaction may take effect after the sample is loaded onto the indexed, barcoded substrate. It will be appreciated that different components of an enzyme mix may be provided to (1) the barcoded substrate, and (2) the sample, other than the specific examples (e.g., a first type of enzyme to the barcoded substrate, and a second type of enzyme to the sample; two types of enzymes to the barcoded substrate, and a catalyst or other reagent to the sample, or vice versa, etc.).

Panels (B)-(D) describe non-enzymatic release mechanisms. In panel (B), the oligonucleotide molecule 985 may be coupled to (e.g., conjugated to) a desthiobiotin moiety 986 (a biotin analog which lacks the sulfur atom), and the bead 981 may be coupled to a streptavidin moiety 988. The desthiobiotin moiety 986 and the streptavidin moiety 988 may be bound together to couple the oligonucleotide molecule 985 to the bead 981, though at less binding strength (e.g., with disassociation constant (K_{d}) on the order of 10⁻¹¹ M) than that between streptavidin and biotin moieties (e.g., with K_{d} on the order of 10⁻¹⁵ M). Upon provision of a high concentration of biotin moieties 987, the biotin-streptavidin bonds may displace the desthiobiotin-streptavidin bonds to release the oligonucleotide molecule 985 from the bead 981. Such non-enzymatic release mechanisms may be beneficial over enzymatic release mechanisms. For example, it may result in shorter release time (faster than enzymatic cleavage time, e.g., using USER cleave); it may reduce cost (biotin is cheaper compared to enzyme reagents); it may improve diffusion in hydrogel environments as biotins are much smaller in size than enzymes; it may reduce waste by permitting recycling of streptavidin-coupled beads (by extracting the biotin from the used beads, and attaching new desthiobiotin-conjugated oligonucleotide molecules).

In panel (C), the first strand 982 may comprise one or more azobenzene (denoted ‘X’) and a cleavage site, denoted “U”, at or adjacent to the 5′ terminus. For example, the cleavage site may comprise 1, 2, 3, 4, or more uracil residues. Azobenezene is a light-sensitive molecule that changes between a trans-form and a cis-form under certain light and/or heat conditions. For example, azobenzene changes from trans-form to cis-form under UV light, and changes from cis-form to trans-form under VIS light and/or heat. Azobenzene may enable fast photoswitch of hybridization states of at least a segment of two strands of nucleic acid molecules. Azobenzene may be incorporated between the nucleotides of the first strand. The melting temperature (Tₘ) between the first strand 982 and the second strand 983 may be significantly reduced when the azobenzene is in cis-form, as compared to the Tₘ between the two strands without azobenzene and as compared to the Tₘ when the azobenzene is in trans-form, as it weakens the hydrogen bonds between the two strands. The Tₘ between the first strand the second strand may not vary as much when the azobenzene is in trans-form as compared to the Tₘ between the two strands without the azobenzene. Thus, providing UV light stimulus (e.g., 365 nm) to the bead 981 may change the azobenzene to cis-form and de-hybridize, destabilize, or facilitate de-hybridizing or destabilizing of the two strands at the location of the azobenzene incorporations. With reference to the methods described with respect to FIG. 8, after the substrate is indexed, the beads may be subjected to a USER enzyme mix, as described elsewhere herein, to form nicked strands (e.g., on strand 982) at the cleavage sites. The bead may be subjected to UV light to trigger the azobenzene, resulting in fast release of the nicked strands from the bead.

In panel (D), the oligonucleotide molecule 985 may be coupled to (e.g., conjugated to) an azobenzene moiety 992 at a 5′ terminus, which azobenzene moiety is hydrophobic, and the bead 981 may be coupled to (e.g., conjugated to) an alpha-cyclodextrine (a-CD) moiety 991, which is a barrel protein with a hydrophilic outer shell and a hydrophobic core. The photoswitchable trans-form and cis-form of azobenzene has been described elsewhere herein. In trans-form, azobenzene may enter the a-CD core via hydrophobic interaction, and thus bind the oligonucleotide molecule 985 and the bead 981 in a non-covalent bond. In cis-form, azobenzene may exit the a-CD core and detach from the a-CD, thus releasing the oligonucleotide molecule 985 from the bead 981. The transition from trans-form to cis-form of azobenzene may be triggered by UV light.

The methods described herein may further comprise methods for attenuation or prevention of long distance diffusion by reagents, such as by attenuating diffusion altogether or by attenuating diffusion along a certain direction(s) on the substrate, to retain spatial information of a sample (after diffusion begins). It may be undesirable for reagents to diffuse too far in a direction that is along an axis or plane contained in a final spatial map generated (e.g., x-y plane) as it may confuse proximity data that is later used to reconstruct the spatial map. The methods may prevent small particles that tend to diffuse relatively fast (e.g., DNA), compared to the duration of various reactions described herein (e.g., barcode release by USER enzyme, capture of analytes, etc.), from diffusing too far from an originating location before tagging of the analyte occurs, increasing the accuracy of a final spatial map. In some cases, diffusion can be attenuated by adding viscous reagents (e.g., PEG, etc.) and/or modulating one or more other reaction conditions (e.g., temperature).

In some cases, diffusion can be attenuated by encapsulating a reaction space in a gel, hydrogel (e.g., PEG hydrogel), or other mesh or matrix (e.g., polymer mesh matrix) to hinder particle movement therethrough. The encapsulation may be reversible. For example, the mesh or matrix may be degradable, such as after a certain period of time and/or upon application of one or more stimuli (e.g., chemical stimulus to induce, e.g., hydrolysis, enzymatic stimulus, photo stimulus, etc.). In an example, the reaction space comprising the sample and beads is crosslinked with a hydrophilic polymer to create a mesh that attenuates diffusion throughout the reaction space. The mesh may be nanoscale. In an example, a 4-arm PEG-acrylate macromer and PEG-dithioglycolate crosslinker is used to form a PEG hydrogel that is degradable. In some cases, protein (e.g., bovine serum albumin (BSA) protein) or other solutes may be embedded or entrapped within the mesh network to increase a crowding effect to further attenuate diffusion. A pore size may be tuned by selecting component of changing length (molecular weight). Examples include tetra-acrylate PEG, cross-linked by thiol-PEG-thiol elements.

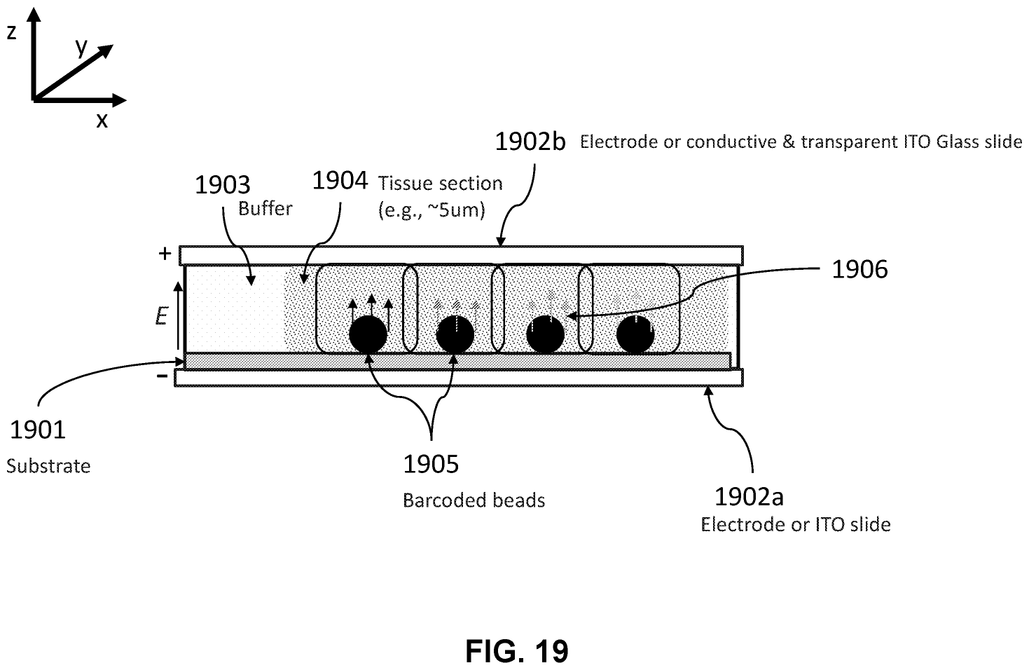

Alternatively or in addition, the reaction space may be subjected to electrophoresis to accelerate movement of charged particles (e.g., DNA, mRNA, spatial tags, etc.) along a direction of the electric field, such as along the z-axis when an x-y plane spatial map is generated, to attenuate diffusion along non-z-axis directions. FIG. 19 illustrates a schematic for subjecting the reaction space to electrophoresis. The beads 1905, each comprising the releasable oligonucleotide molecules 1906, and sample 1904 (e.g., 5 micron tissue) may be loaded onto a substrate 1901 as described elsewhere herein. An appropriate buffer 1903 (e.g., (Tris base/acetic acid/EDTA (TAE), Tris/borate/EDTA (TBE), etc.) may be added between two electrodes 1902a and 1902b that sandwich the sample-loaded substrate to facilitate electrophoresis. An electrode (conductive material) may be or comprise an indium tin oxide (ITO) slide. In some cases, the electrode is substantially transparent and conductive. The electric field may be activated with or prior to release of the oligonucleotide molecules 1906 of the beads 1905. In some cases, the electric field may be activated after release of the oligonucleotide molecules. The released oligonucleotide molecules may be directed to diffuse primarily along a direction of the electric field (e.g., along z-axis), instead of in the x- or y-axis directions. In an example, mRNA analytes and barcode oligonucleotide molecules move toward the positive electrode. The electric field may be maintained until substantial completion of all tagging reactions. FIG. 19 illustrates a box around each bead to represent the diffusion cloud of the oligonucleotide molecules (in the x-z plane). A reaction space may be both encapsulated in a mesh or matrix (e.g., PEG hydrogel) as described elsewhere herein and subjected to electrophoresis.

Enrichment may be facilitated by various capture mechanisms, such as a pair of a capture entity and a capturing entity. The capture entity may comprise or be biotin, a capture sequence (e.g., nucleic acid sequence) which may be hybridized to a strand of the oligonucleotide molecule or which may be part of another nucleic acid molecule conjugated to the oligonucleotide molecule, a magnetic particle capable of capture by application of a magnetic field, a charged particle capable of capture by application of an electric field, a combination thereof, or one or more other mechanisms configured for, or capable of, capture by a capturing entity. The capturing entity may comprise or be streptavidin when the capture moiety comprises biotin, a complementary capture sequence when the capture entity comprises a capture sequence, an apparatus, system, or device configured to apply a magnetic field when the capture entity comprises a magnetic particle, an apparatus, system, or device configured to apply an electrical field when the capture entity comprises a charged particle, a combination thereof, and/or one or more other mechanisms configured to capture the capture entity. In some instances, the capturing group may comprise a secondary capture entity, for example, for subsequent capture by a secondary capturing entity. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic bead) and the secondary capturing entity can comprise a magnetic system (e.g., magnet, apparatus, system, or device configured to apply a magnetic field, etc.). In some instances, the secondary capture entity can comprise a charged particle (e.g., charged bead carrying an electrical charge) and the secondary capturing entity can comprise an electrical system (e.g., magnet, apparatus, system, or device configured to apply an electric field, etc.). In some instances, the capture moiety comprises biotin, the capturing moiety comprises streptavidin coupled to a secondary capture entity, a magnetic bead, and the secondary capturing entity comprises a magnetic system.

### In Situ Capturing of Analytes

In some instances, an analyte may be captured in situ for recording a location before further processing.

In some instances, an analyte may be detected by a probe. In some cases, detecting an analyte by a probe may comprise capturing the analyte by the probe. In some cases, capturing an analyte of a biological sample may comprise contacting the biological sample with a probe linked to a barcode. In some cases, a probe may comprise a barcode to record and encode a location of a captured analyte (e.g., location within a sample). In some instances, a probe may be immobilized on a solid support. The solid support may comprise any of the substrates described elsewhere herein. The solid support, in some cases, may comprise a slide. In some cases, a slide may comprise an array. The slide may be a glass slide or be composed of other material. In some cases, the solid support may comprise a plurality of probes. For example, a slide may comprise a plurality of probes. In some cases, each probe on a solid support may be arranged uniformly. For example, each probe may be separated from each other by a uniform distance. Alternatively, the probes may be arranged non-uniformly. In some cases, each probe may be arranged in a rectangular grid. Other arrangements, such as a circular grid or any customized format, may also be used in some instances. A probe immobilized on the solid support may be or comprise any of the binders or linkers described herein. For example, a surface of the substrate may be modified to comprise the probes, or the probes may be added as an additional layer or coating on the substrate. Where the solid support is a slide, the slide may be integrated as a part of the substrate surface or otherwise coupled (e.g., as another layer) to the substrate. The substrate may be substantially planar, as described elsewhere herein.

In other instances, an analyte may be bound to a probe, and that probe may be captured by a binding molecule, such as any of the binders or linkers described elsewhere herein.

In some instances, a location may comprise a coordinate in an absolute coordinate reference system. In some cases, a Cartesian coordinate system may be used as a coordinate for an analyte. A Cartesian coordinate of an analyte, in some instances, may comprise an x and y coordinate on a two dimensional plane. In some cases, a Cartesian coordinate of an analyte may comprise an x, y, and z coordinate on a three dimensional plane. In some cases, a spatial coordinate may refer to the location on a solid support where a barcode is immobilized on. For example, in a two dimensional image of an array slide comprising a plurality of barcodes, each spatial coordinate may represent each location where each barcode sequence is located on the array slide.

In some instances, a molecular label encoding a location may be attached to a substrate. In some cases, a molecular label encoding a location may be immobilized on a substrate, such as directly onto the surface of the substrate, or via an intermediary medium such as a solid support. For example, the molecular label may be immobilized to the solid support, which solid support is immobilized on the substrate. In some cases, the intermediary medium (e.g., solid support) may comprise a bead, a glass slide, an array slide, a gel bead, a magnetic bead, a microwell plate, or any combination thereof. An array slide, in some instances, may comprise a microarray slide. The substrate may comprise a plurality of individually addressable locations, as described elsewhere herein.

In some instances, a plurality of individually addressable locations may be smaller than the biological sample being analyzed such as that information extracted from the plurality of individually addressable locations may be used to identify one sample. In some cases, a biological sample bigger than one substrate comprising a plurality of individually addressable locations may be analyzed by more than one such substrate for analysis. For example, at least two substrates, each comprising a plurality of individually addressable locations, may be required to cover a biological sample comprising a plurality of analytes. Each substrate may contact a non-overlapping or overlapping section of the biological sample, contacting or capturing a non-overlapping or overlapping subset of analytes. The result obtained from the two substrates may be combined to reconstruct the spatial location of each analyte in the biological sample.

In some instances, a molecular label encoding a location may be attached to an individually addressable location on a substrate. In some instances, a probe comprising a barcode sequence may be attached to an individually addressable location on a substrate. In some instances, a probe comprising a unique barcode sequence may be attached or immobilized to an individually addressable location on a substrate. In some cases, a first probe comprising a first unique barcode sequence may be attached or immobilized to a first individually addressable location while a secondary probe comprising a second unique barcode sequence may be attached or immobilized to a second individually addressable location on a substrate. In some instances, a substrate may be planar. The surface of a substrate may be substantially planar. The surface of the substrate may be patterned or textured, such as to comprise a plurality of wells. The surface of a substrate, in some cases, may also comprise any cross-sectional surface profiles described herein, such as but not limited to those in FIGS. 3A-3G. Patterning of the surface of a substrate, in some instances, may comprise surface chemistry. In some cases, any surface chemistry described herein may be used to pattern the surface of a substrate.

In some instances, a substrate may have any shape as described herein. In some cases, a shape of a substrate may comprise a regular polygon or an irregular polygon. A polygon, in some cases, may comprise a triangle, square, rectangle, quadrilateral, pentagon, hexagon, heptagon, octagon, nonagon, or decagon. A polygon, in other cases, may comprise a number of sides. A number of sides for a polygon, in some cases, is 3, 4, 5, 6, 7, 8, 9, 10, 11, 12, 13, 14, 15, 16, 17, 18, 19, or 20. In some cases, a shape of a substrate may be an ellipse. An ellipse, in some cases, may comprise a circle or an oval.

In some instances, each barcode sequence of a probe may comprise a unique molecular identity. For example, each barcode sequence may comprise a unique nucleotide sequence. Each unique nucleotide sequence may allow each barcode sequence to be distinguished from other barcode sequences. In some cases, the identity of each barcode sequence is pre-determined as the probe is immobilized. In other cases, the identity of each barcode sequence is determined after the probe is immobilized. Other identities based on unique physical, chemical, or biological attributes may also be used as a molecular identity.

In some instances, a barcode sequence on a substrate may be part of a probe. In some cases, a barcode sequence on a substrate may be linked to a probe. Such linkage may comprise a covalent or a non-covalent bond. For example, a probe may comprise a barcode sequence and a capture sequence. For example, a probe may comprise a barcode sequence and an oligo(dT) primer sequence. The oligo(dT) primer sequence may capture an endogenous mRNA sequence. In some cases, other capture sequences may be used. For example, a capture sequence that can capture an intron, nucleosome, or peptide sequence. In one case, a capture sequence may comprise a sequence specific to one group of genes, transcripts, or other nucleic acid species) (e.g., genes or transcripts sharing a similar sequence). Such a capture sequence may enrich the group of genes, transcripts, or other nucleic acid species captured. In some instances, the capture of an analyte in a biological sample by a probe with a barcode may link the analyte to the barcode. Linking the analyte to the barcode, in some cases, may record or assign the location of the barcode to the analyte in the biological sample being captured. For example, if mRNA A is captured by a probe with a barcode encoding location B but not C, mRNA A is encoded with location B but not C. In some cases, a capture sequence and a barcode sequence may comprise the same modalities (e.g., nucleic acid sequence). In other cases, a capture sequence may be replaced by other modalities or moieties for capturing an analyte. Other modalities or moieties for capturing an analyte may comprise an antibody, affinity tag, peptide, lipid, carbohydrate, any derivatives thereof, or any combination thereof. For example, a probe may comprise a nucleic acid barcode sequence (e.g., DNA) and an affinity peptide (e.g., an antibody). Other modalities or moieties for capturing an analyte, in some instances, may also comprise an organic or inorganic compound. In some cases, the modalities or moieties for capturing an analyte may enrich the species of analytes being analyzed. For example, a capture modality comprising an antibody targeting a type of proteins may the type of proteins.

In some instances, a substrate may comprise a plurality of sets of probes, each detecting one type of analytes. For example, a substrate may comprise both oligo(dT) primer sequence probes to capture endogenous mRNA sequences and nucleosome-targeting sequence probes to capture nucleosomes. In some cases, probe sequences targeting different types of analytes with the same location may have the same barcode sequence.

In some instances, a substrate may comprise a cluster of probes with barcode sequences for each type of probe. In some cases, a cluster of probes with barcode sequences may comprise about 1×10¹⁰ molecules, about 1×10¹ molecules, about 1×10² molecules, about 1×10³ molecules, about 1×10⁴ molecules, about 1×1⁵ molecules, about 1×10⁶ molecules, about 1×10⁷ molecules, about 1×10⁸ molecules, about 1×10⁹ molecules, about 1×10¹⁰ molecules, about 1×10¹¹ molecules, about 1×10¹² molecules, about 1×10¹³ molecules, about 1×10¹⁴ molecules, about 1×10¹⁵ molecules, about 1×10¹⁶ molecules, about 1×10¹⁷ molecules, about 1×10¹⁸ molecules, about 1×10¹⁹ molecules, or about 1×10²⁰ molecules.

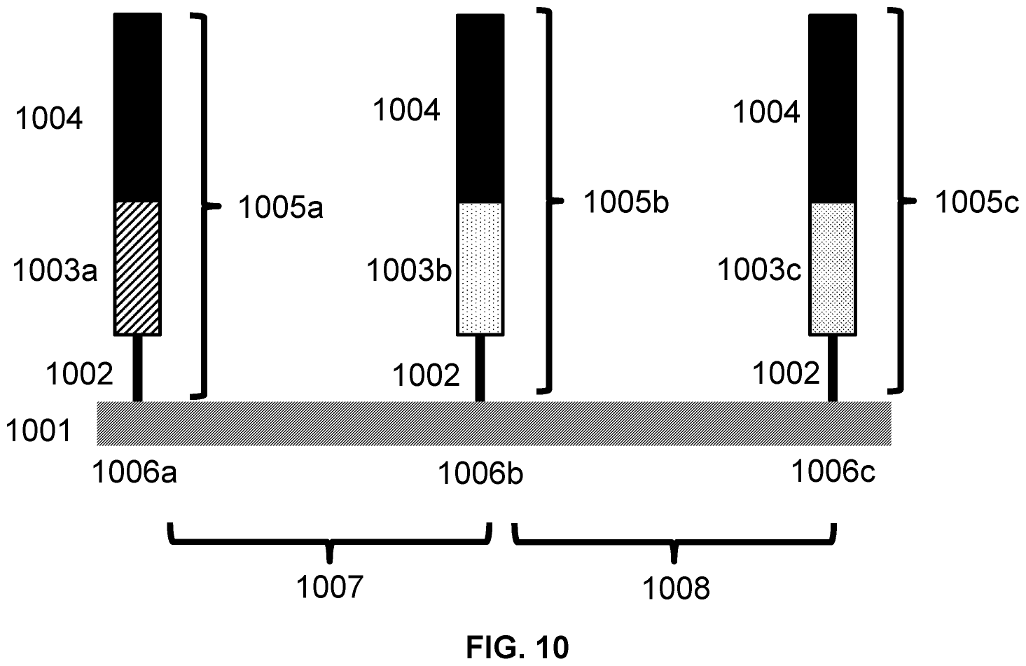

FIG. 10 shows an example substrate with probes at addressable locations for capturing analytes from different spatial locations. Three probes 1005a, 1005b, and 1005c are attached to a substrate 1001 (e.g., slide, wafer, etc.) at individually addressable locations 1006a, 1006b, and 1006c, respectively. Individually addressable locations 1006a and 1006b are separated by a distance 1007, and individually addressable locations 1006b and 1006c are separated by a distance 1008. Distances 1007 and 1008 may be the same or may vary depending on the application. Each probe is attached to substrate 1001 via linker 1002. In some cases, the linker 1002 may be cleavable by a stimulus (e.g., physical or chemical stimulus). Each probe contains capture sequence 1004 (e.g., oligo-dT sequences). Capture sequence 1004 may vary to capture different types of analytes such as protein, polypeptide, carbohydrate, or lipids. At each addressable location, each probe also contains a unique barcode sequence. For example, probe 1005a at individually addressable location 1006a contains barcode sequence 1003a. Probe 1005b at individually addressable location 1006b contains barcode sequence 1003b. Probe 1005c at individually addressable location 1006c contains barcode sequence 1003c. Each barcode may encode various types of information. For example, the barcode sequence 1003a may comprise a spatial barcode specific to individually addressable location 1006b. The barcode sequence 1003a may comprise a unique molecular identifier. The barcode sequence 1003a may comprise a sample or analyte identifier. While this example describes the probe as a nucleic acid, comprising a nucleic acid capture sequence (e.g., 1004) and a nucleic acid barcode sequence (e.g., 1003a-c), it will be appreciated that the probe may comprise non-nucleic acids configured to capture any type of analyte, and the barcode sequence may be replaced with other methods of encoding (e.g., dye, tag, etc.). As will be appreciated and described elsewhere herein, the probes may be immobilized to the substrate via an intermediary substrate (e.g., beads, slides, etc.).

In some instances, a biological sample is treated by a fixative, or fixated, before contacting the probe. Fixation, in some cases, may render the location of an analyte invariable. For example, an mRNA molecule may not diffuse away from its location in a tissue after fixation. Contacting the fixed tissue to a substrate (e.g., an array slide, wafer, etc.) with a plurality of probes with barcodes may allow the probe to capture the endogenous mRNA at its fixed location in the tissue. In some cases, a permeabilization step of a fixed biological sample may facilitate the contacting between the probe and the analyte. In some cases, a permeabilization of a biological sample may release an analyte. For example, permeabilization of a fixed tissue may release an mRNA vertically downward, e.g., via gravity, so that it can contact the probe. Once captured, the endogenous mRNA is encoded with the location information encoded by the barcode sequence.

In some instances, a biological sample may be contacted with more than one set of probes, wherein each set of probes is immobilized on a different substrate or different regions of a same substrate. In some cases, a biological sample may be contacted with two sets of probes. The two sets of probes may detect or capture the same analyte. In some cases, detecting the same analyte by two sets of probes may facilitate an analysis of the analyte. For example, an analyte may be assigned to an addressable location with higher confidence level if the analyte is detected by two sets of probes assigned to the same addressable location on two different substrates (or to corresponding addressable locations on two different regions of a same substrate), compared to an analyte that is only detected by one set of probes. In some instances, two sets of probes of two different substrates assigned to the same addressable location (or same corresponding addressable locations) may detect or capture different analytes. For example, a first substrate or first substrate region may comprise oligo(dT) primer sequence probes to capture endogenous mRNA sequences. A second substrate or second substrate region may comprise nucleosome-targeting sequence probes to capture nucleosomes. In some cases, probe sequences targeting different types of analytes with the same location may have the same barcode sequence.

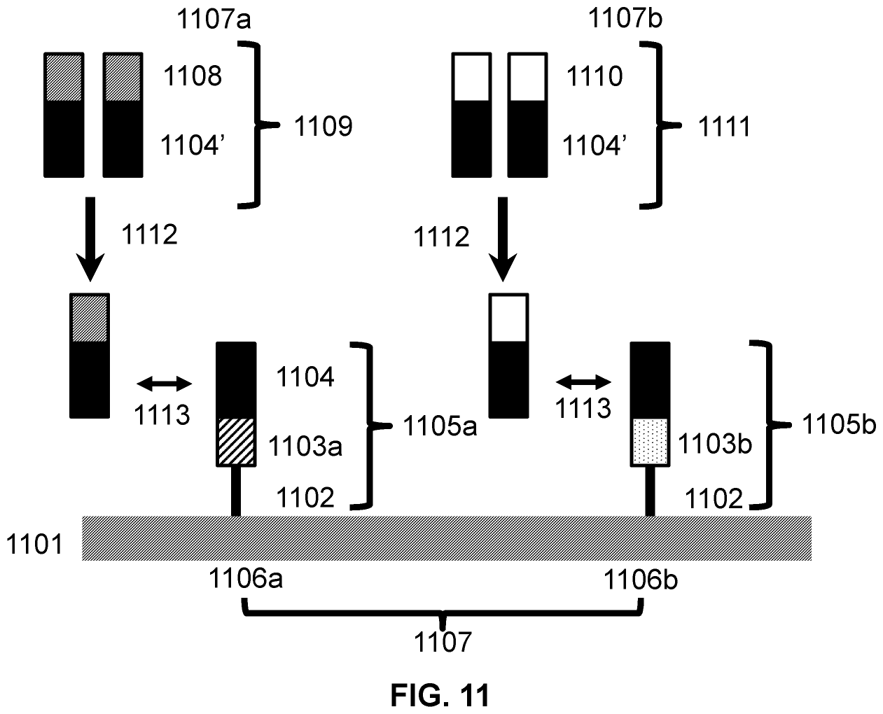

FIG. 11 shows an example method of using an array with probes to capture endogenous mRNAs with spatial resolution. Two probes 1105a and 1105b are attached to substrate 1101 at individually addressable locations 1106a and 1106b, respectively. Individually addressable locations 1106a and 1106b are separated by a distance 1107. Each probe is attached to substrate 1101 (e.g., wafer, array slide, etc.) via linker 1102. In some cases, the linker 1102 may be cleavable by a stimulus (e.g., physical or chemical stimulus). Each probe contains capture sequence 1104 (e.g., oligo-dT sequences). Probe 1105a at individually addressable location 1106a contains barcode sequence 1103a. Probe 1105b at individually addressable location 1106b contains barcode sequence 1103b. A biological sample may comprise mRNAs 1109 and 1111 at endogenous locations 1107a and 1107b in the biological sample. mRNAs 1109 and 1111 contain coding sequences 1108 and 1110, respectively. mRNAs 1109 and 1111 also share poly-A sequence 1104′ that can bind to the capture sequence 1104. When the biological sample is permeabilized, mRNAs 1109 and 1111 will be released to contact probes 1105a and 1105b, respectively, such as by gravity 1112. Binding of probe 1105a-mRNA 1109 and probe 1105b-mRNA 1111 at individually addressable locations 1106a and 1106b will barcode mRNAs 1109 and 1111 with barcode sequences 1103a and 1103b, respectively, by a downstream reaction such as a reverse transcription reaction and/or an amplification reaction (e.g., RCA amplification). Once barcoded, the extension product and/or amplification product can be cleaved at linker 1102 to facilitate downstream processing. In some cases, a probe may be released from the substrate prior to coupling with the analyte (e.g., mRNA). The distance that the probe may travel between post-release and analyte capture may be limited, thus retaining spatial information. As will be appreciated and described elsewhere herein, the probes may be immobilized to the substrate via an intermediary substrate (e.g., beads, slides, etc.).

In some instances, a probe linked to an analyte may be released from a solid substrate. In some cases, a probe may be cleavable or otherwise releasable. In some cases, a probe may comprise a cleavable linker. In some cases, a probe may be cleavable by a stimulus (e.g., physical or chemical stimulus). The probe may be released prior to, during, or subsequent to binding to an analyte. In some cases, once a probe binds to an analyte(s), the probe may be released from the solid substrate by cleaving the probe. In some cases, a probe may be released from a solid substrate once the probe has a link with or otherwise encodes the spatial information location to an analyte. In some cases, a captured analyte may be released from a probe once the analyte is encoded with a spatial location. For example, a bead comprising a probe that encodes spatial location (e.g., via a barcode sequence on an oligonucleotide molecule) may be contacted with an analyte to link the probe with the analyte (e.g., a target nucleic acid may hybridize to the probe), and optionally the complex may be further processed (e.g., subjected to an extension reaction), after which the probe-analyte complex, or derivative thereof, is cleaved from the substrate (e.g., wafer, bead, etc.).

In some instances, a probe may be used in a downstream reaction after capturing an analyte. For example, an oligo(dT) primer sequence may become a primer for a reverse transcription reaction once it captures an mRNA molecule. In some instances, a downstream reaction may not proceed or be triggered until a reagent is supplied. In some cases, a reagent to trigger a downstream reaction may be supplied by the systems and methods described herein or thereof. For example, a reverse transcription may not proceed after the oligo(dT) sequence captures an mRNA molecule until the nucleotides, enzymes, buffers, or any combination thereof are provided to the hybridized oligo(dT)-mRNA sequences. In some instances, a downstream reaction may create derivatives of an analyte. Such derivatives may facilitate other processing, such as but not limited to, an amplification or sequencing reaction. For example, an amplification may comprise a reverse transcription, primer extension, PCR, LCR, helicase-dependent amplification, asymmetric amplification, RCA, RPA, LAMP, NASBA, 3SR, HCR, MDA, derivatives thereof, or any combination thereof. An amplification may also be a linear or non-linear amplification. In one case, a reverse transcription of an mRNA molecule may create a cDNA molecule, wherein the cDNA molecule may be amplified, e.g., by PCR or RCA, and sequenced by a sequencer. In some cases, a sequencing reaction may be carried out as described herein (e.g., Examples).

In some instances, the identity of an endogenous mRNA or a linked barcode sequence may be derived by the identity of its derivatives. For example, the sequence of a cDNA molecule derived from a mRNA molecule and a probe with a barcode sequence may identify the nucleotide sequence of the mRNA molecule and the barcode sequence. In some instances, an analyte, its barcode sequence, or derivatives thereof may be processed or analyzed in the same reaction (e.g., a sequencing reaction). In other cases, an analyte, its barcode sequence, or derivatives thereof may be processed or analyzed in different reactions.

In some instances, a biological sample may be dissected, dissociated, digested, or degraded after an analyte is encoded with the location information. In some cases, a biological sample may be dissected, dissociated, digested, or degraded after a probe captures an analyte and a barcode is linked to the analyte. Dissection, dissociation, digestion, or degradation of a biological sample may facilitate the processing of the processing of an analyte. Once linked with a barcode, in some cases, the location of an analyte may be retained and decoded afterwards. For example, dissection, dissociation, digestion, or degradation of a biological sample may not remove the encoded location information of an analyte. The location of each analyte may be reconstructed digitally by decoding the barcode of the analyte.

In some instances, a biological sample may be aligned with the contacted solid support with a plurality of barcode sequences to facilitate the mapping of the analytes to their locations in the biological sample. For example, a tissue is contacted with a substrate comprising a plurality of probes comprising oligo(dT) capture primers and barcode sequences, arranged in a rectangular grid, to capture the mRNA molecules. The tissue is then imaged to align the rectangular grid on the tissue. Since the location and sequence of each barcode on the rectangular grid is known, the location of the mRNA molecules in the tissue can be identified by decoding the barcode sequences and overlaying them on the imaged tissue with the rectangular grid.

In some instances, a probe may be attached to beads. In some cases, probes attached to beads may be dispensed on a substrate (e.g., a wafer, a glass slide, or a microwell plate). The probe-bound beads may be dispensed in a similar manner as sample-bound beads are dispensed, as described elsewhere herein. In some cases, dispensing probes attached to beads on a solid support may facilitate an increase of the density of the probes compared to that when the probe is attached directly to a slide or glass slide. Such an increase in density may be 1-fold, 10-fold, 100-fold, 100-fold, 1,000-fold, 10,000-fold, or more.

In some instances, dispensing beads with probes on a substrate may create a random distribution of probes with different barcode sequences on the solid support. In some instances, beads comprising probes with different barcode sequences may be deposited into microwells of a microwell plate. In some cases, the addressable location of a barcode sequence of a probe may not be pre-determined when a bead comprising the probe is dispensed or deposited. In some cases, the addressable location of a barcode sequence of a probe may be determined once a bead comprising the probe is dispensed on a substrate. For example, the nucleotide sequence of a barcode sequence may be identified by sequencing. Such sequencing may comprise Sequencing by Oligonucleotide Ligation and Detection (SOLiD). In some cases, a sequencing reaction may be carried out as described herein (e.g., Examples). In some cases, the nucleotide sequence of a barcode may be identified by sequential rounds of fluorescence hybridizations. For example, each round of hybridization may comprise contacting a set of additional probes. An additional probe, in some cases, may comprise an optical moiety. In some cases, an additional probe may also comprise a fluorescent dye. In some cases, an additional probe may comprise a complementary nucleotide sequence to a probe on a substrate. Each additional probe may hybridize to a barcode sequence or a portion thereof on the probe. An image may be taken, and the solid support stripped for another round of hybridization. Since each barcode may be designed to comprise a unique set of hybridization patterns in the sequential rounds of hybridizations, each location for each barcode may be determined. In some cases, the barcode may also be identified by the methods and compositions described herein (e.g., the methods and compositions described in “Fluorescent hybridization”).

In some instances, any staining technologies or fluorescent hybridization methods disclosed and thereof herein may be applied in combination with technologies such as Slide-seq to enable high throughput on-substrate sample processing. The general principle of the method is similar to those described in Rodrigues et al., 2009, Science 363 (6434): 1463-1467, which is hereby incorporated herein in its entirety. In some cases, a biological sample (e.g., a tissue sample) may be positioned on previously immobilized beads where each immobilized bead bears surface primers with a particular barcode sequence or a combination of barcode sequences. The surface may be the probe described elsewhere in this disclosure. Implementation in the current system, however, may include aspects that drastically enhance processing scale and efficiency. As disclosed herein, beads may include any number of barcode sequence combinations, depending on the scale of the analysis and number of potential target transcripts. For example, beads made with different barcode sequences may be obtained by mixing different groups of beads, each bead within a group has surface primers that include the same barcode sequence. In some cases, between 2,000 and 50 billion beads may be immobilized on a substrate. In some cases, surface primers on the beads may include dozens, hundreds, thousands, tens and hundreds of thousands, up to millions or hundreds of millions, or more of different barcode sequences. Any embodiments contemplated herein may be applied in connection with the particular implementation. In some cases, surface primers on a bead may comprise only one barcode sequence. In some cases, a bead may comprise a plurality of different barcode sequences. In some cases, a bead may comprise surface primers including two or more tandemly connected barcode sequences; e.g., two, three, four, five, six, seven, eight, nine, or ten or more barcode sequences, which may be the same or difference on the same bead. For example, each barcode sequence segment may be connected during a combinatorial indexing or combinatorial barcoding scheme to generate a diverse population of final barcode sequences. Alternatively, the barcode sequence segments may be connected in non-combinatorial schemes, such as in bulk. The barcode sequence, in segments, in combination, or in sub-combination, may comprise one or more types of barcode sequences, such as one or more of a sample barcode (e.g., unique to a sample), a bead barcode (e.g., unique to a bead), a unique molecular identifier (UMI) barcode (e.g., unique to a molecule), etc. In some cases, two adjacent barcode sequences may be connected via a pre-defined sequence. In such cases, one of the barcode sequences may be a sample barcode sequence specifying the sample source.

For example, beads may be divided into sub-groups and dispensed onto different locations on the substrate. In each sub-group, the surface primers on the beads may include the same sample barcode sequence, and connected to one or more additional barcode sequences (e.g., random sequences, primer sequences, unique molecular identifier sequences, functional sequences, etc.). Once sequences of the surface primers are determined, the beads may be mapped onto the substrate based on their respective locations (e.g., based on optical or fluorescent imaging) and barcode sequences. Subsequently, tissue sections may be transferred onto the immobilized beads. In some cases, the tissue sections may comprise a thin sheet of the specimen, e.g., comprising only a single layer of cells. The cells in the tissue may be lysed in a permeabilization step. An analyte in the cells may be released during the permeabilization step to contact the surface primer.

In some instances, a probe may be used in a downstream reaction after capturing an analyte. For example, an oligo(dT) primer sequence may become a primer for a reverse transcription reaction once it captures an mRNA molecule. In some instances, a downstream reaction may not proceed or be triggered until a reagent is supplied. In some cases, a reagent to trigger a downstream reaction may be supplied by the systems and methods described herein or thereof. For example, a reverse transcription may not proceed after the oligo(dT) sequence captures an mRNA molecule until the nucleotides, enzymes, buffers, or any combination thereof are provided to the hybridized oligo(dT)-mRNA sequences. In some instances, a downstream reaction may create derivatives of an analyte. Such derivatives may facilitate other processing, such as but not limited to, an amplification or sequencing reaction. For example, an amplification may comprise a reverse transcription, primer extension, PCR, LCR, helicase-dependent amplification, asymmetric amplification, RCA, RPA, LAMP, NASBA, 3SR, HCR, MDA, derivatives thereof, or any combination thereof. An amplification may also be a linear or non-linear amplification. In one case, a reverse transcription of an mRNA molecule may create a cDNA molecule, wherein the cDNA molecule may be amplified by PCR or RCA and sequenced by a sequencer. In some cases, a sequencing reaction may be carried out as described herein (e.g., Examples).

In some instances, a biological sample may be dissected, dissociated, digested, or degraded after an analyte is encoded with a barcode sequence specifying a spatial location. In some cases, a biological sample may be dissected, dissociated, digested, or degraded after a probe captures an analyte and a barcode sequence is linked to the analyte. Dissection, dissociation, digestion, or degradation of a biological sample may facilitate the processing of the processing of an analyte. Once linked with a barcode sequence, in some cases, the location of an analyte may be retained and decoded afterwards. For example, dissection, dissociation, digestion, or degradation of a biological sample may not remove the encoded location information of an analyte. The location of each analyte may be reconstructed digitally by decoding the barcode of the analyte.

In some cases, fluorescent imaging analysis may be performed prior to the cells being lysed. Fluorescent labels or fluorescent dyes may be used to identify cell types or other properties of individual cells based on, for example, specific cell surface markers. In some cases, cell specific markers may comprise proteins such as proteins, glycoproteins, phosphorylated proteins. In some cases, small molecules or macromolecules (e.g., antibodies) that specifically bind to specific cell surface markers may be used to locate cells and identify cell types. In some cases, two or more markers may be used in combination. For example, labeled antibodies that are raised against specific markers may be used to locate and identify specific types of cells.

In some instances, the fluorescent image analysis may be performed on a selected subset of a biological sample. In some cases, the fluorescent image analysis may be performed on a selected subset of cells or tissue sections. In some cases, a biological sample may be divided into a plurality of sub-samples. In some cases, a substrate may be divided in a plurality of sections. In other cases, a biological sample may be divided into a plurality of sections if the biological sample is in contact with a substrate. In one case, a section of a substrate may comprise a sub-sample of a biological sample. In some cases, each of the plurality of sections may be labeled by a section-specific labeling agent. Such a section-specific labeling agent may comprise a surface marker of a cell. Such a surface marker for section-specific labeling may be identified by immunostaining or fluorescent in-situ hybridization. Such a surface marker for section-specific labeling may also be identified by methods and compositions described here (e.g., the methods and compositions described in “Fluorescent hybridization”). In some instances, the results of the analyses described thereof may be combined with a section of a substrate, wherein the section comprises beads, surface primers, or barcode sequences being mapped on the section of the substrate based on the locations and identities of the barcode sequences.

In some instances, at least about 100 sections may be processed simultaneously by systems and methods disclosed thereof. In some cases, at least about 200 sections may be processed simultaneously by systems and methods disclosed thereof. In some cases, at least about 300 sections may be processed simultaneously by systems and methods disclosed thereof. In some cases, at least about 400 sections may be processed simultaneously by systems and methods disclosed thereof. In some cases, at least about 500 sections may be processed simultaneously by systems and methods disclosed thereof. In some cases, at least about 600 sections may be processed simultaneously by systems and methods disclosed thereof. In some cases, at least about 700 sections may be processed simultaneously by systems and methods disclosed thereof. In some cases, at least about 800 sections may be processed simultaneously by systems and methods disclosed thereof. In some cases, at least about 900 sections may be processed simultaneously by systems and methods disclosed thereof. In some cases, at least about 1000 sections may be processed simultaneously by systems and methods disclosed thereof. In some cases, at least about 1100 sections may be processed simultaneously by systems and methods disclosed thereof. In some cases, at least about 1200 sections may be processed simultaneously by systems and methods disclosed thereof. In some cases, at least about 1300 sections may be processed simultaneously by systems and methods disclosed thereof. In some cases, at least about 1400 sections may be processed simultaneously by systems and methods disclosed thereof. In some cases, at least about 1500 sections may be processed simultaneously by systems and methods disclosed thereof. In some cases, at least about 1600 sections may be processed simultaneously by systems and methods disclosed thereof. In some cases, at least about 1700 sections may be processed simultaneously by systems and methods disclosed thereof. In some cases, at least about 1800 sections may be processed simultaneously by systems and methods disclosed thereof. In some cases, at least about 1900 sections may be processed simultaneously by systems and methods disclosed thereof. In some cases, at least about 2000 sections may be processed simultaneously by systems and methods disclosed thereof. In some cases, at least about 2100 sections may be processed simultaneously by systems and methods disclosed thereof. In some cases, at least about 2200 sections may be processed simultaneously by systems and methods disclosed thereof. In some cases, at least about 2300 sections may be processed simultaneously by systems and methods disclosed thereof. In some cases, at least about 2400 sections may be processed simultaneously by systems and methods disclosed thereof. In some cases, at least about 2500 sections may be processed simultaneously by systems and methods disclosed thereof. In some cases, at least about 2600 sections may be processed simultaneously by systems and methods disclosed thereof. In some cases, at least about 2700 sections may be processed simultaneously by systems and methods disclosed thereof. In some cases, at least about 2800 sections may be processed simultaneously by systems and methods disclosed thereof. In some cases, at least about 2900 sections may be processed simultaneously by systems and methods disclosed thereof. In some cases, at least about 3000 sections may be processed simultaneously by systems and methods disclosed thereof. In some cases, systems and methods disclosed herein can process dozens, hundreds, or thousands of standard tissue sections (e.g., at the same time, including but not limited to about 20, 50, 100, 200, 300, 500, 600, 800, 1,000, 1,500, 2,000, 3,000, 7,000, 10,000 or more. In some embodiments, tens of thousands or more tissue sections can be processed. As described elsewhere in this disclosed, a standard tissue section can be of a size that is comparable to tissue size founded in conventional Formalin-Fixed, Paraffin-Embedded (FFPE) tissue slides.

In some cases, a plurality of sections of a substrate may be reconstructed after the fluorescent image analysis. Reconstruction of the substrates, in some cases, may reconstruct a biological sample. The reconstructed biological sample may retain the same biological context of the biological sample before the reconstruction. Such a biological context, in some cases, may comprise the composition of the biological sample, the location of each constituent of the biological sample, the identity of each constituent of the biological sample, or any combinations thereof. For example, a tumor sample comprising a heterogeneity of cancer cell types may undergo the fluorescent image analysis described in this disclosure. Each cell or subset of cells of the tumor sample may be processed by the fluorescent image analysis simultaneously. After the fluorescent image analysis, the analyzed cells or subsets of cells of the tumor may be reconstructed to represent the cellular context of the tumor, i.e., the heterogeneity of the cancer cell types of the tumor sample. In other cases, a biological sample may comprise more than one tissue. For example, a tumor sample may comprise cancer cell and normal cells. In other cases, a tumor sample may comprise a tumor and a normal tissue.

In some instances, a biological sample may be processed while on a substrate. In some cases, a biological sample may be processed while immobilized on a substrate. In some cases, a reagent may be dispensed over the biological sample using the systems described in this disclosed. For example, a reagent for permeabilization may be dispensed on a tissue on an immobilized substrate. The cells in the tissue may then be lysed on the substrate. In other cases, other reagents, such as those for sequencing; fluorescent hybridizations; or other described methods that can identify the spatial barcode of an analyte, the sections of the substrate, the sub-samples of a biological sample, or the cells of the biological sample may also be dispensed on the substrate or biological sample. Beneficially, the on-substrate processing can make reagent distribution highly efficient, and obviate the need to transfer a sample or analyte from one container (or location) to another container (or location). In some cases, a reagent may be dispensed over at least about 1000, at least about 5000, at least about 10000, at least about 50000, at least about 100000, at least about 500000, at least about 1000000, at least about 5000000, at least about 10000000, at least about 50000000, at least about 100000000, at least about 500000000, at least about 1000000000, at least about 5000000000, at least about 10000000000, or at least about 50000000000 addressable locations of substrate. In some cases, a reagent may be dispensed over at least about 1000, at least about 5000, at least about 10000, at least about 50000, at least about 100000, at least about 500000, at least about 1000000, at least about 5000000, at least about 10000000, at least about 50000000, at least about 100000000, at least about 500000000, at least about 1000000000, at least about 5000000000, at least about 10000000000, or at least about 50000000000 analytes of a substrate. In some cases, a reagent may be dispensed over at least about 1000, at least about 5000, at least about 10000, at least about 50000, at least about 100000, at least about 500000, at least about 1000000, at least about 5000000, at least about 10000000, at least about 50000000, at least about 100000000, at least about 500000000, at least about 1000000000, at least about 5000000000, at least about 10000000000, or at least about 50000000000 analytes of a biological sample. In some cases, a reagent may be dispensed over at least about 1000, at least about 5000, at least about 10000, at least about 50000, at least about 100000, at least about 500000, at least about 1000000, at least about 5000000, at least about 10000000, at least about 50000000, at least about 100000000, at least about 500000000, at least about 1000000000, at least about 5000000000, at least about 10000000000, or at least about 50000000000 sections of a substrate. In some cases, a reagent may be dispensed over at least about 10, at least about 50, at least about 100, at least about 500, at least about 1000, at least about 5000, at least about 10000, at least about 50000, at least about 100000, at least about 500000, at least about 1000000, at least about 5000000, at least about 10000000, at least about 50000000 cells of a tissue.

In some instances, the systems and methods disclosed herein may quantify the number of a plurality of analytes. In other cases, the systems and methods disclosed herein may quantify the number of a plurality of cells. In some cases, the systems and methods disclosed herein also may the number of a plurality of sub-samples or sections. Each of the plurality of analytes or cells may comprise a marker. Such a marker may comprise a chemical moiety. In some cases, the marker may comprise a chemical moiety present in the analytes or cells. The chemical moiety may comprise a cell surface marker described elsewhere in the disclosure. In other cases, a marker may also comprise a barcode sequence described elsewhere in the disclosure.

In some instances, the determination of the location of barcode sequences and the identification of analytes may be carried out in multiple biological samples. For example, a first tissue may contact a substrate with a plurality of beads with probes to capture the mRNA in the tissues, and a second tissue may contact the same solid support to identify the location of each probes. In some cases, multiple biological samples for the determination of the location of barcodes and the identification of analytes may be highly similar. For example, a first section of tissues may be used for the determination of the location of barcode sequences, and an immediately adjacent section of tissues may be used for the identification of analytes.

In some instances, a protein or peptide may be used to spatially barcode an analyte. Such a protein or peptide, in some cases, may comprise an enzyme. In some cases, an enzyme may be endogenous. In other cases, an enzyme may be recombinant. Such a recombinant enzyme may comprise domain or sequence that determines its location. In some cases, a location may have a subcellular resolution. In some cases, a recombinant enzyme may be localized to different subcellular compartments comprising plasma membrane, nucleus, endosome, Golgi bodies, ER, nucleolus, lysozyme, mitochondria, cytoskeleton, centriole, ribosome, stress granules, RNA granules, cytoplasm, vacuole, or chloroplast. In some instances, an enzyme may process an analyte. Such an enzyme may comprise the APEX2 enzymatic domain or activity. In some cases, an enzyme with the APEX2 domain or activity, when supplied with biotin-phenol and hydrogen peroxide, may couple a biotin molecule to an RNA molecule. In some cases, the amount of processing an analyte undergoes may depend on the amount of enzyme in vicinity. For example, a recombinant APEX2 enzyme may only process nearby RNA molecules but not those away from the enzyme. In some cases, an analyte having processed by an enzyme may suggest that the RNA molecule shares a similar location as the enzyme. For example, an RNA molecule labeled with a biotin molecule by a nuclear recombinant APEX2 may suggest that the RNA molecule localizes to the nucleus. In some instances, a labeled analyte may be purified before further processing (e.g., sequencing). For example, an RNA molecule labeled with a biotin molecule may be purified using streptavidin beads. In some instances, a labeled analyte may not be purified before further processing. A purified labeled analyte may undergo a reaction. Such a reaction may comprise a nucleic acid amplification described herein. In some cases, a labeled analyte may be identified by a sequencing reaction. In some cases, a sequencing reaction may be carried out as described herein (e.g., Examples).

### In Situ Sequencing

In some instances, an analyte or its derivative of a biological sample may be screened while it remains in its endogenous context or structure of the biological sample. In some cases, an analyte or its derivative of a biological sample may be screened without being dissociated from its endogenous biological context or structure in the biological sample. In some cases, an analyte or its derivative of a biological sample may be screened while other molecules in the biological sample are removed from the same biological context or structure in the biological sample. For example, a nucleic acid molecule or its derivative may be sequenced while it is still attached to the tissue where it originates from. In some instances, screening an analyte or its derivative of a biological sample while it remains in its endogenous biological context or structure may retain the location of the analyte of the biological sample.

In some instances, an analyte may be converted to a derivative. In other cases, an analyte may be converted to a series of derivatives. In some cases, an analyte may be converted to a first derivative, and the first derivative may then be converted to a second derivative. For example, an RNA molecule may be converted to a cDNA molecule via a reverse transcription. In another example, an RNA molecule may be converted to a cDNA molecule via a reverse transcription, and the cDNA molecule may be converted to an RCA product via an RNA amplification.

In some instances, an analyte or its derivative may be processed. In some cases, a processing may comprise binding an analyte or its derivative with a probe. A probe may comprise a nucleic acid, a polypeptide, a carbohydrate, a chemical, derivatives thereof, or any combinations thereof. A nucleic acid probe, in some cases, may comprise a linear or circular nucleic acid probe. In some cases, a linear nucleic acid probe may comprise a padlock probe. A padlock probe, in some cases, may comprise a gapped padlock probe.

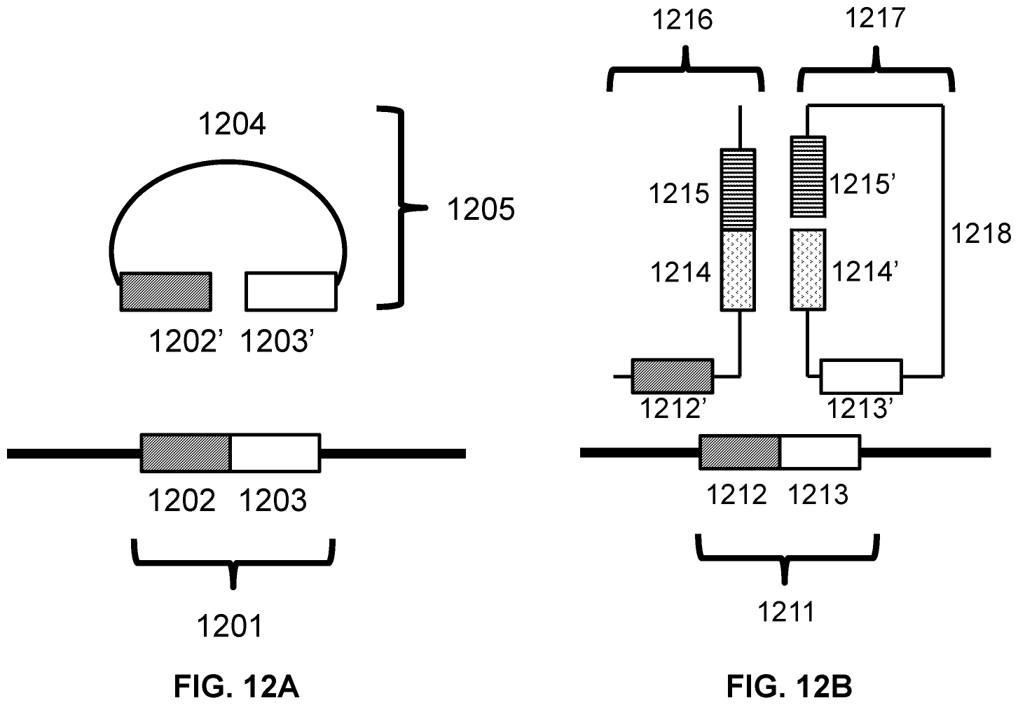

In some instances, a binding of a probe with an analyte or its derivative may initiate or trigger a downstream reaction. In some cases, a binding of a padlock probe with a target nucleic acid molecule (e.g., cDNA molecule) may initiate or trigger a downstream reaction. FIG. 12A shows an example padlock probe for detection of an analyte. Target 1201 may comprise target sequence 1202 and 1203 located adjacent to each other at one end. Padlock probe 1205 may comprise sequence 1202′ and 1203′ that can bind to target sequences 1202 and 1203, respectively. Sequences 1202′ and 1203′ are arranged in an opposite orientation with respect to that of target sequences 1202 and 1203 such that their binding will bring the two ends of padlock probe 1205 in proximity. Such proximity may allow a first downstream reaction such as a ligation or nucleotide extension. Padlock probe 1205 also contains sequence 1204 that may contain sequences that is detectable by a second downstream reaction such as hybridization with a fluorescent labeled probe. Both the first and second downstream reaction can facilitate the detection of padlock probe 1205.

In some instances, a binding of a first region of a padlock probe with a target nucleic acid molecule and a binding of a second region of the probe with another nucleic molecule may initiate or trigger a downstream reaction. Such a nucleic acid molecule may comprise a second probe. In some cases, a downstream reaction may only be triggered when at least three bindings occur: a binding between a first region of a padlock probe with a first region of a target nucleic acid molecule; a binding between a second region of the target nucleic acid molecule and a first region of a second probe; and a binding between a second region of the second probe and a second region of the padlock probe.

In some instances, a downstream reaction may comprise a chemical reaction within a probe, within an analyte or its derivative, between a probe and an analyte or its derivative, or any combination thereof. In some cases, a downstream reaction may comprise an extension reaction between two hydroxyl ends of a probe. In some cases, a downstream reaction may comprise a circularization reaction between two hydroxyl ends of a probe. In some cases, a downstream reaction may comprise a ligation reaction between two hydroxyl ends of a probe. For example, padlock probe may undergo a ligation or a nucleotide extension between its two hydroxyl ends upon binding to its target nucleic acid molecule. In other cases, a padlock probe may undergo a ligation between its two hydroxyl ends when a first region of the padlock probe binds to a first region of a nucleic acid molecule; a second region of the nucleic acid molecule binds to a first region of a primer; and a second region of the primer binds to a second region of the padlock probe.

FIG. 12B shows an example padlock probe set for detection of an analyte. Target 1211 may comprise target sequences 1212 and 1213 located adjacent to each other at one end. A first probe 1217 contains sequence 1213′ that can bind to target sequence 1213. A second probe 1216 contains sequence 1212′ that can bind to target sequence 1212. The above-described binding events bring probes 1216 and 1217 in proximity. The second probe 1216 contains sequences 1214 and 1215. The first probe 1217 contains sequences 1214′ and 1215′ that can bind to sequences 1214 and 1215, respectively. Since sequences 1214′ and 1215′ are orientated in an opposite direction to that of sequences 1214 and 1215, their binding will bring sequences 1214′ and 1215′ in proximity. The binding of sequences 1212-1212′, 1213′1213′, 1214-1214′, and 1215-1215′ will trigger a downstream reaction such as an extension and/or an amplification (e.g., an RCA amplification) of the first probe 1217 and facilitate its detection by hybridization with a fluorescent labeled probe using sequence 1218.

In some instances, a downstream reaction may comprise an amplification reaction. In some cases, a downstream reaction may comprise a nucleic acid amplification reaction. For example, an amplification may comprise a reverse transcription, primer extension, PCR, LCR, helicase-dependent amplification, asymmetric amplification, RCA, RPA, LAMP, NASBA, 3SR, HCR, MDA, derivatives thereof, or any combination thereof. An amplification, in some instances, may also be a linear or non-linear amplification. In some cases, a nucleic acid analyte may be amplified to form nanoballs for further processing. A nanoball, in some cases, may be single stranded or double stranded.

In some instances, an analyte or its first derivative may be modified to a second derivative during a conversion reaction described herein. In some cases, a target nucleic acid molecule or its first derivative may be modified to a second derivative during an amplification reaction. One such modification may comprise a chemically modified base (e.g., an amine-modified base) during the amplification. In some cases, a modification to a second derivative may facilitate embedding of the second derivative to the endogenous context or structure of its original target nucleic acid molecule. In some cases, a modification to a second derivative may facilitate a cross-linking of the second derivative to the cellular protein matrix. In some cases, amine-based modification may link in situ synthesized second derivative to a cellular polymer at the endogenous location of its original target nucleic acid molecule. In some cases, a modification to a second derivative may also facilitate removal of other undesirable molecules. Such undesirable molecule may comprise any molecules other than the second derivative (e.g., proteins or lipids). In some instances, an analyte may be removed from a biological sample or degraded after being processed. In some cases, an analyte may be removed from a biological sample or degraded after being converted to a derivative. For example, an RNA molecule may be removed after it is converted to a cDNA molecule via a reverse transcription reaction. In other cases, a first derivative of an analyte may be removed from a biological sample or degraded after being modified to a second derivative. In some cases, an analyte may be removed from a biological sample or degraded after being converted to a derivative. In some cases, even after an analyte or a first derivative of the analyte is removed, a subsequent derivative of the analyte or the first derivative may retain the endogenous location of the analyte.

In some instances, an analyte or its derivative in a biological sample may be determined by a sequencing reaction using methods and systems as described herein (e.g., Examples) while the analyte or the derivate remains in its endogenous location the biological sample. For example, a cDNA molecule, an RCA amplification product, or a nanoball, converted from an RNA molecule in a tissue sample, may be sequenced. In some cases, an analyte or its derivative in a biological sample may also be determined by sequencing-by-synthesis (SBS) or its derivatives while the analyte or the derivate remains in its endogenous location the biological sample. In some cases, a biological sample may be processed to allow sequencing using methods and systems as described herein (e.g., Examples) while the analyte or the derivate remains in its endogenous location the biological sample. For example, a tissue sample with a cDNA molecule, an RCA amplification product, or a nanoball may be placed on 310 of FIG. 3 for sequencing.

In some instances, the binding of a probe with an analyte or its derivative may also label an analyte. In some cases, labeling an analyte or its derivative may comprise a probe with a molecular identifier. In some cases, labeling an analyte or its derivative may also comprise a nucleic acid probe (e.g., a primer) with a molecular identifier. Such a molecular identifier, in some cases, may comprise a barcode sequence. In some cases, screening an analyte or its derivative may only comprise screening a molecular identifier of the analyte or the derivative. For example, a nucleic acid probe, each with a unique barcode sequence, may bind to a cDNA molecule derived from an RNA molecule in a tissue sample. A sequencing reaction using methods and systems as described herein (e.g., Examples) may only identify the barcode sequence in the tissue sample to denote the location of the nucleic acid probe in the tissue sample. Such a nucleic acid probe, in some cases, may comprise a linear or circular nucleic acid probe. In some cases, a linear nucleic acid probe may comprise a padlock probe. A padlock probe, in some cases, may comprise a gapped padlock probe.

In some instances, an analyte or its derivative in a biological sample may be removed from its endogenous location in the biological sample after the location of the analyte or the derivative is denoted. Denoting the location of an analyte or its derivative, in some cases, may comprise utilizing a molecular identifier as described herein, such as but not limited to a barcode sequence. In some cases, removal of an analyte or its derivative from its endogenous location in a biological sample may facilitate subsequent processing. Such a subsequent processing, in some cases, may comprise an identification of an analyte or its derivative. In some instances, a molecular identifier may be denoted and subsequently processed to link an analyte or its derivative to an endogenous location in a biological sample. For example, a cDNA molecule or RCA product derived from an RNA molecule in a tissue may be labeled with an oligonucleotide barcode primer during the conversion from the RNA molecule. The oligonucleotide barcode may be sequenced in a first sequencing reaction while it remains in the tissue and its location is denoted. Subsequently, the cDNA molecule or RCA product, comprising the oligonucleotide barcode, may be removed from the tissue and subjected to a second sequencing reaction. The identity of the oligonucleotide barcode and the cDNA molecule or RCA product may then be identified. The cDNA molecule or RCA product may be mapped to its endogenous location by the location of the oligonucleotide barcode denoted in the first round of sequencing reaction.

### Fluorescent Hybridization

In some instances, an identification of an analyte or its derivative at its endogenous location in a biological sample may require a pre-determination or a priori determination of the analyte or the derivative. In some cases, a pre-determination or a priori determination of an analyte may comprise a molecular identifier of the analyte. Such a molecular identifier, in some cases, may comprise an endogenous or an exogenous molecular identifier. In some cases, an endogenous molecular identifier may comprise a unique molecular attribute of an analyte. For example, a unique barcode sequence may be a molecular identifier of a target nucleic acid molecule. In some cases, an exogenous molecular identifier may comprise modifying an analyte. Such a modification may comprise a covalent or non-covalent modification. For example, a probe comprising a unique barcode sequence may bind to a target nucleic acid molecule. Such a probe, in some cases, may comprise attributes for further processing. For example, a probe may comprise sequences that bind to an additional probe, such as a fluorescent-labeled hybridization probe for screening or detecting an analyte or the probe binding to the analyte. In one example, a nucleic acid probe comprising a barcode sequence may be used to bind and hybridize an RNA molecule in a tissue sample in situ. The nucleic probe may comprise any configurations described herein. In some cases, the nucleic acid probe may also trigger any downstream reaction as described herein. In other cases, the nucleic acid probe may also facilitate any conversion of a target nucleic acid molecule to any derivative described herein. For example, the nucleic acid probe may be a primer for a reverse transcription extension to convert an RNA molecule into a cDNA molecule. The nucleic acid probe may also be a primer to amplify a cDNA molecule during a PCR or RCA amplification reaction.

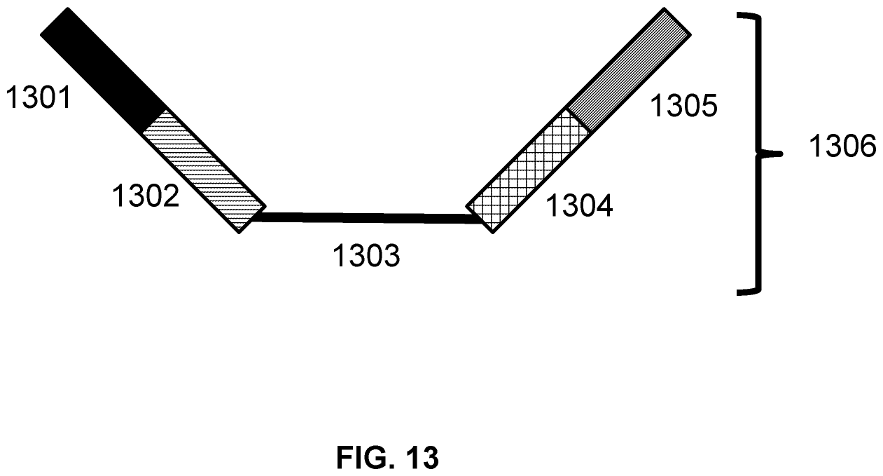

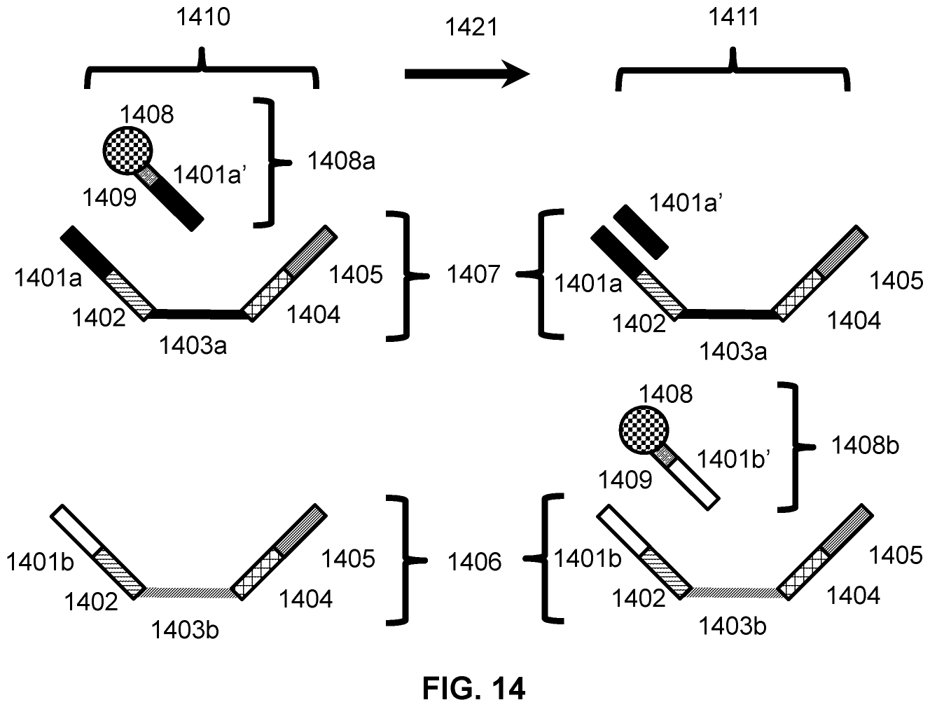

In some instances, an additional probe may comprise one nucleotide sequence for detection. In some cases, an additional probe may comprise two different nucleotide sequences for detection. In some cases, an additional probe may comprise three different nucleotide sequences for detection. In some cases, an additional probe may comprise four different nucleotide sequences for detection. In some cases, an additional probe may comprise 5, 6, 7, 8, 9, or 10 different nucleotide sequences for detection. FIG. 13 shows an example additional probe. Additional probe 1306 contains capture sequence 1303 that can bind to a target nucleic acid sequence. 1303 is flanked by four sets of barcode oligonucleotide sequences 1301, 1302, 1303, and 1304. In some cases, an additional probe may be processed by fluorescent hybridization. In some cases, an additional probe may be processed according to the workflows described with respect to FIG. 14. In some cases, an additional probe may be hybridized by a third probe. The third probe may not comprise a label, such as a fluorescent label. The third probe may be subjected to another round of hybridization with a fourth probe. In some cases, the fourth probe may be labeled by a fluorescent label. In some cases, detection of the third probe by the fourth probe may detect the additional probe. The detection of the third probe, in some cases, may provide a stronger signal than that of the additional probe. In other cases, the additional probe or the third probe may be subjected to any amplification process as described herein before detection. In some instances, an additional probe may be processed with any sequencing methods and systems described herein. In some cases, an additional probe may be processed to allow sequencing using methods and systems as described herein (e.g., Examples). In other cases, an additional probe may also be processed to allow sequencing using methods and systems for in situ sequencing as described herein. In some cases, a barcode oligonucleotide sequences may be a unique molecular identifier.

In some instances, a unique molecular identifier may identify an analyte or its derivative. In some cases, the number of barcode oligonucleotide sequences (B) may equal to the number of target nucleic acid molecules (M) (i.e., B=M). In other cases, B may be less than M (i.e., B<M). In some cases, a number of iterations of tagging (t) with barcode oligonucleotide sequences may create a number of unique combinations of barcode oligonucleotide sequences (C) such that B^{t}=C. In some cases, a unique combination of barcode oligonucleotide sequences may identify target nucleic acid molecules (i.e., B^{t}=C>=M). For example, with 4 sets of tagging, each set comprising 10 unique barcode oligonucleotide sequences, 10⁴=10,000 unique combinations of barcode oligonucleotide sequences may be created to label 10,000 target nucleic acid molecules. In some cases, B may be more than M (i.e., B>M). Having more B than M, in some cases, may facilitate error-correction. For example, a probe with a unique barcode oligonucleotide sequence may bind to a nucleic acid molecule other than a pre-determined target nucleic acid molecule. However, two probes, each with a unique barcode oligonucleotide sequence, may only bind to one pre-determined target nucleic acid molecule and not any other nucleic acid molecules.

In some instances, a first set of molecular identifiers may be connected to a second set of molecular identifiers. In some instances, a first set of barcode oligonucleotide sequences may be continuous with a second set of barcode oligonucleotide sequences. For example, the 3′ end of a first barcode oligonucleotide sequence may be directly adjacent to the 5′ end of a second barcode oligonucleotide sequence. In some cases, a first set of barcode oligonucleotide sequences may not be connected to a second set of barcode oligonucleotide sequences. In other cases, a first set of barcode oligonucleotide sequences may not be continuous with a second set of barcode oligonucleotide sequences. For example, the 3′ end of a first barcode oligonucleotide sequence to the 5′ end of a second barcode oligonucleotide sequence may be separated by at least one nucleotide.

In some instances, identifying an analyte may comprise fluorescent labeling a nucleic acid probe bound to the analyte with an additional probe. In some cases, a unique fluorescent label may identify a probe with a barcode. In some cases, the number of unique fluorescent labels (F) may equal to B (i.e., F=B). In other cases, F may be less than B (i.e., F<B). In some cases, a number of pseudo-fluorescent labeling rounds (L) of F may create a number of unique pseudo-fluorescent labels (P) such that F×L=P. Such a unique pseudo-fluorescent label may not differ from another unique pseudo-fluorescent label by fluorescence or colorimetric measurement. In some cases, each round of pseudo-fluorescent labeling may differ from another round of pseudo-fluorescent labeling temporally or sequentially. For instance, a fluorescent label may be measured at time point 1 in a first round of pseudo-fluorescent labeling, and the same fluorescent label may be measured at time point 2 in a second round of pseudo-fluorescent labeling. The first and second rounds of pseudo-fluorescent labeling may create two pseudo-fluorescent labels distinguishable by at which time point a measurement is taken, even though the two pseudo-fluorescent labels have the same fluorescence or colorimetric measurement at each time point. In some cases, a unique pseudo-fluorescent label may identify a unique barcode oligonucleotide sequence (i.e., F×L=P=B). For example, one unique fluorescent label may create 10 pseudo-fluorescent labels to label 10 unique barcode oligonucleotide sequences.

In some instances, each round of pseudo-fluorescent labeling may comprise contacting and hybridizing an additional probe comprising a fluorescent label to a probe bound to an analyte (e.g., a target nucleic acid molecule); and removing the probe or the additional probe from the analyte. In some cases, removing a fluorescent label may comprise stripping a biological sample chemically to release the probe or the additional probe. In some cases, removing a fluorescent label may comprise a chemical cleavage reaction of a probe or additional probe. For example, the link between a fluorescent label and a probe or additional probe may be cleaved in a chemical cleavage reaction of the probe or additional probe. In one case, a mild reducing agent may be used in a chemical cleavage reaction to cleave a disulfide link between a fluorescent label and a probe or additional probe. One such mild reducing agent may comprise Tris(2-carboxyethyl)phosphine (TCEP). In other cases, removing a fluorescent label may comprise specifically degrading the probe or the additional probe. For example, a DNase (e.g., DNase I) may be used to degrade a DNA probe bound to an RNA molecule in a tissue sample. In some cases, removing a fluorescent label may comprise extinguishing the fluorescent signal (e.g., quenching the fluorescent signal) of the fluorescent label. In one example, extinguishing the fluorescent of the fluorescent label may comprise photobleaching.

FIG. 14 shows an example pseudo-fluorescent labeling process. Additional probes 1406 and 1407 have two different target nucleic acid binding sequence 1403a and 1403b, respectively. 1406 and 1407 share three barcode oligonucleotide sequences 1402, 1404, and 1405. 1406 has a unique barcode oligonucleotide sequence 1401b while 1407 has another unique barcode oligonucleotide sequence 1401a. A first hybridization round 1410 comprises hybridizing a first detection probe 1408a with 1406 and 1407. 1408a contains fluorescent label 1408, disulfide link 1409, and sequence 1401a′ that can bind to 1401a but not 1401b. In the first hybridization round, 1408a will bind and detect 1407. 1406 and 1407 will then undergo a chemical cleavage reaction 1421 to remove 1408 from 1408a, leaving 1401a′ bound to 1401a. In a second hybridization 1411, a second detection probe 1408b is used to hybridize 1406 and 1407. 1408b contains 1408, disulfide link 1409, and sequence 1401b′ that can bind to 1401b but not 1401a. In the second hybridization round, 1408b will bind and detect 1406. By recording the fluorescent labeled hybridization using the same fluorescent label 1408 in 1410 and 1411, the same fluorescent label creates two pseudo-fluorescent labels to detect two additional probes.

In some instances, pseudo-fluorescent labeling and sets of unique molecular identifier (e.g., unique barcode oligonucleotide sequence) tagging may be used together to detect a plurality of analytes (e.g., target nucleic acid molecules). In one example, 10,000 different target RNA molecules may be identified by 40 unique DNA barcode oligonucleotide sequences and 4 unique fluorescent labels. 10,000 DNA probes may bind 10,000 different target RNA molecules. Each DNA probe comprise 4 sets of barcode oligonucleotide sequences. Each set of barcode oligonucleotide sequences comprises 10 different unique barcode oligonucleotide sequences. Each set of unique barcode oligonucleotide sequences may be detected by 10 additional probes in 10 sequential hybridization rounds. Each additional probe, while sharing the same fluorescent label, may only bind to 1 of each set of barcode oligonucleotide sequences in the DNA probe. Each hybridization round may comprise hybridizing an additional probe to the DNA probe and removing the additional probe by fluorescent quenching, thereby creating a unique pseudo-fluorescent label for each barcode oligonucleotide sequence of the DNA probe. By repeating the hybridization rounds 4 times (for the 4 sets of barcode oligonucleotide sequences), 10,000 RNA sequences may be identified by 40 unique barcode oligonucleotide sequences and 4 unique fluorescent labels. In some instances, detecting a fluorescent label or pseudo-fluorescent label may comprise methods and systems, or derivatives, as described herein (e.g., Example 1, 5, or 6).

### Spatial Isolation of Analytes

In some instances, the location of an analyte may be determined by spatial specific isolation of the analyte. In some cases, an analyte located at a specific location of a biological sample may be isolated from the other analytes not located at the same location. In other cases, a subset of analytes sharing a similar location may be isolated together. For example, a thin section of a tissue may be dissected from the rest of tissue. In some instances, an isolated subset of analytes sharing a similar specific location may be identified in bulk. In other cases, an isolated subset of analytes may be divided into further subsets. For example, a thin section of tissue may be further into individual cells, wherein each cell is processed in partition. In some cases, an isolated subset of analytes may undergo further processing. Such a processing may comprise tagging the subset of analyte with a barcode sequence described herein, with or without a substrate. For example, the mRNA molecules from each thin section of a tissue may be linked with a unique barcode sequence. The barcode may encode the location for each mRNA molecule. In some cases, different subsets of analytes may be processed in bulk after each subset is linked with a barcode sequence.

In some instances, an analyte or subset of analytes sharing the same location may not comprise any molecular label identifying its location. In some cases, the location of an analyte or subset of analytes may not need to be encoded or decoded when the analyte or subset of analytes is identified in isolation. For example, the nucleic acid molecules from the anterior section of an embryo may be isolated from the section and sequenced in a sequencing reaction. The sequencing reads obtained from such sequencing reaction may be identified as the anterior section of the embryo.

In some instances, a series of subsets of analytes from a biological sample, each with a determined specific location, may be reconstructed to represent the original spatial distribution of the analytes in the biological sample. In some cases, a reconstructed spatial distribution of analytes in a biological sample may comprise one axis. In other cases, a reconstructed spatial distribution of analytes in a biological sample may comprise two axes. In some cases, a reconstructed spatial distribution of analytes in a biological sample may comprise three axes.

In some cases, for each axis of spatial distribution of analytes, a series of subsets of analytes may be isolated along that axis. For example, to reconstruct the anterior-posterior distribution of mRNA molecules in an embryo, a series of section of embryo along the anterior-posterior axis may be isolated. The mRNA molecules from each section may then be isolated and identified. In other instances, to reconstruct the spatial distributions of analytes on more than one axis, the analytes identified on one axis may overlap with those of the other axis. In some cases, a reconstructed spatial distribution of analytes may comprise one axis. In some cases, a reconstructed spatial distribution of analytes may comprise two axes. In some cases, a reconstructed spatial distribution of analytes may comprise three axes.

In some instances, a sample may be divided into a series of biological samples. In some cases, a biological sample may be a subset of a sample. Such a sample may be a tissue, organ, or a group of cells. In some cases, a sample may be divided into a plurality of biological samples through sectioning, wherein each cut section may be one of the plurality of biological samples. In one example, a three-dimensional tissue may be cut and divided into multiple layers. In some cases, the order, location, or geographical reference to other biological samples of a biological sample may be recorded. Such a record may be used to align the plurality of biological samples. In some instances, a plurality of analytes in each of the plurality of biological samples maybe may be encoded with a spatial location using the methods and compositions described herein. In some cases, the spatial location of each analyte in a sample may be reconstructed by combining the spatial location of each analyte in each of the plurality of biological samples and the alignment of the plurality of biological samples according to the order of the biological samples.

In some instances, a series of subsets of a biological sample, each comprising a group of analytes localizing at a specific location, is isolated. In some cases, a subset of a biological sample may be dissected. In some cases, dissecting a subset of a biological sample may comprise laser capture microdissection (LCM). In some cases, LCM may comprise using a laser beam to dissect and isolate a subset of a biological sample. Such a subset of biological sample may comprise a tissue region of a tissue. In some cases, multiple LCM may be performed to dissect and isolate multiple subsets of a biological sample. For example, a tissue sample may be divided into multiple tissue regions, wherein each region may be isolated by LCM. LCM, in some cases, may be combined with other analyte screening method. For example, LCM may be combined with single cell RNA-sequencing to identify the spatial distribution of single cell transcriptomes of a tissue.

In some instances, a subset of a biological sample may be isolated by a dissociation process. A dissociation process may comprise an enzymatic digestion reaction. Such an enzymatic digestion reaction may comprise a protease. In some instances, a dissociation process may comprise a limited dissociation process to preserve endogenous structure. In some cases, a subset of biological sample under limited dissociation process may comprise analytes that physically interact or share a location. In one case, a tissue may be digested in a condition that prevents an enzyme to be fully active. Such a condition may comprise incubating the digestion reaction in a suboptimal pH, temperature, ionic strength, time, quantity of enzyme, or any combinations thereof.

In some cases, a subset of a biological sample may express a marker. A marker, in some cases, may be endogenous or exogenous. In some cases, a marker may be constitutive active or situational active. For example, a marker may comprise a photoactivatable green fluorescent protein (GFP). Only a group of cells of at a specific location may express the photoactivatable GFP. Such a group of cells may be isolated by any dissection or dissociation methods described herein.

### Spatial Markers for Analytes

In some instances, the location of a group of analytes may be determined by a marker. In some cases, the location of a group of analytes may be determined by more than one marker. In some cases, the spatial distribution of a group of analytes may be determined by 1-10, 5-15, 10-20, 15-25, 20-30, 25-35, 30-40, 35-45, 40-50, or more markers. In some instances, an analyte of a group of analytes may be used as a marker for the group of analytes.

In some instances, a group of analytes of a biological sample and a marker for the biological sample may be analyzed using the same biological sample. In some cases, a group of analytes of a biological sample and a marker for the biological sample may be analyzed using two biological samples. The two biological samples, in some cases, are highly similar. In some cases, one assay may be used to analyze a group of analytes and its marker. In other cases, more than one assay may be used to analyze a group of analytes and its marker. In some cases, analyzing a group of analytes may comprise one type of assays and analyzing the marker of the group of analytes may comprise another type of assays. For example, in single cell transcriptomic analysis of an embryo, each single cell transcriptome may be analyzed by single cell mRNA sequencing. Parallelly, a marker gene for the individual single cell transcriptomes may be analyzed by in situ hybridization using a second embryo.

In some instances, a marker may be assessed qualitatively or quantitatively. A qualitative assessment of a marker, in some cases, may comprise the presence or absence of a marker. In some cases, a marker may be assessed quantitatively. A quantitative assessment of a marker, in some cases, may comprise the level of a marker. For example, a marker gene may be assessed for its presence or absence in a cell or a tissue. A marker gene may also be assessed for its expression level in a cell or a tissue. In some cases, a quantitative assessment may be converted to a qualitative assessment. In some cases, a quantitative-to-qualitative assessment conversion may comprise a cutoff. For example, a marker gene exceeding a pre-determined expression may be considered to be present in a sample and vice versa.

In some instances, the spatial distribution of a group analytes may be determined using the spatial distribution of a marker or markers. For example, in single cell transcriptomic analysis of an embryo, the transcriptome of each cell of an embryo may be determined by a sequencing reaction using methods and systems as described herein (e.g., Examples). In a parallel assay, the markers for each single cell are determined by in situ hybridization. The qualitative or quantitative difference for each marker is used to identify the single cell transcriptome based on its expression level in the transcriptome. If a marker gene identifies a single cell transcriptome to a location, every other transcript of that transcriptome may be determined to share the same location.

The systems, devices, and methods of the present disclosure may facilitate highly efficient spatial screening applications, such as the various methods described herein. Accordingly, a method for spatial screening may comprise (a) providing a substrate comprising a cell or tissue sample thereon, such as any of the substrates described elsewhere herein, (b) subjecting the substrate to rotation to distribute one or more reagents (e.g., probes, additional probes, etc.) to the cell or tissue sample, and (c) using a detector in optical communication with the cell or tissue sample on the substrate to detect an analyte of the cell or tissue sample. Another method for spatial screening may comprise (a) providing a substrate comprising a cell or tissue sample thereon, (b) distributing one or more reagents to the cell or tissue sample, and (c) using a detector in optical communication with the cell or tissue sample on the substrate to detect an analyte of said cell or tissue sample, during rotation of said substrate.

The analyte may be located within the cell or tissue sample, or on a surface thereof. The cell or tissue sample may be immobilized on the substrate. The cell or tissue sample may be fixed and/or permeabilized on the substrate. In some cases, the cell or tissue sample may be immobilized on top of a probe array immobilized on the surface of the substrate. In some cases, one or more sets of probes may be delivered to the cell or tissue sample prior to, during, or subsequent to immobilizing the cell or tissue sample on the substrate. The probes may be delivered to analyte(s) within the cell or tissue sample. In some methods, one type of analyte may be detected in (c). In some methods, multiple types of analytes may be detected in (c). The probes may be targeted towards a specific analyte (e.g., containing a specific sequence) or a specific type of analyte. The probes may comprise a barcode sequence. The probes may comprise a padlock probe. The probes may comprise multiple types of probes. The probe may be configured to hybridize and/or ligate to or adjacent to an analyte, or derivative thereof (e.g., an extension product, an amplification product, etc.). The probe may comprise an optical moiety, such as a dye (e.g., fluorescent dye). In some cases, all types of probes may comprise the same type of dye. In some cases, different types of probes may comprise different types of dyes.

The detection of the analyte may yield identifying of an identity and/or a location of the analyte in the cell or tissue sample. For example, the location may comprise a 2D and/or 3D location. The identity of the analyte may comprise a sequence and/or type of the analyte.

The reagent dispensing and detecting may be performed using the systems, methods, and devices described herein. For example, the substrate to which the cell or tissue sample is immobilized may be subjected to rotation, prior to, during, or subsequent to reagent dispensing and/or detecting. Rotation in conjunction with reagent dispensing may facilitate highly efficient dispersal of reagents to the cell or tissue sample (or multiple samples) on the substrate. Rotation during detection may facilitate highly efficient detection. Further the various dispensing mechanisms disclosed herein, such as use of dedicated or independent dispense nozzles for each reagent, may prevent fluid contamination upstream of dispensing. Further the open substrate system may provide significantly higher flexibility and higher control for dispensing of a reagent (or sample or other object) to a specific location on the substrate, which is useful for such spatial screening applications.

The substrate of the systems, devices, and methods described herein may comprise an array of plurality of individually addressable locations, that can be associated with the cell or tissue sample immobilized thereto, that can be used to provide high spatial resolution.

### Systems & Kits

Disclosed herein are systems, kits, and reagents that can be used for or in conjunction with the systems and methods described herein. A kit may comprise any reagent described herein. A system may comprise any kit and/or reagent described herein. A system may comprise a state in which the provided kit has not been used, has been used, or is being used.

For example, a kit may comprise substrates, beads, and/or probes for capturing analytes. A kit may comprise any substrate described herein, such as (i) a substrate that does not have any beads immobilized thereto, (ii) a barcoded substrate comprising beads immobilized thereto, the beads comprising barcode sequences, and/or (iii) an indexed, barcoded substrate comprising beads immobilized thereto, the beads comprising barcode sequences. The kit may comprise indexed data comprising identities and locations of the barcode sequences on the indexed, barcoded substrate. For example, the indexed data may comprise sequencing data of the indexed, barcoded substrate. A kit may comprise a plurality of beads comprising barcode sequences. A kit may comprise a plurality of oligonucleotide molecules comprising barcode sequences. A kit may comprise a plurality of oligonucleotide molecules comprising probe and/or capture sequences. A kit may comprise a plurality of oligonucleotide molecules each comprising both a barcode sequence and a capture sequence. A kit may comprise any sequencing reagent described herein. A kit may comprise any amplification reagent described herein.

In one example, a kit comprises (1) a first substrate comprising a plurality of beads immobilized to a plurality of individually addressable locations on the substrate, wherein the plurality of beads comprises oligonucleotide molecules coupled thereto, wherein the oligonucleotide molecules comprise barcode sequences; (2) indexed data comprising identities and locations of the barcode sequences on the first substrate; and (3) a second substrate comprising a second plurality of individually addressable locations configured to immobilize a second plurality of beads. In some cases, the kit further comprises the second plurality of beads that are not immobilized to the second substrate. In some cases, the kit further comprises a reagent configured to release the oligonucleotide molecules from the plurality of beads. The reagent can comprise one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene. In some cases, the kit further comprises sequencing reagents, such as single-base nucleotide mixtures (e.g., A, C, G, T or U) or multi-base nucleotide mixtures (e.g., A&C, A&T, A&C&G, etc.). A single-base or multi-base nucleotide mixture may comprise a mixture of labeled and unlabeled nucleotides. A single-base or multi-base nucleotide mixture may comprise non-terminated nucleotides, in some cases comprising only non-terminated nucleotides (vs terminated nucleotides). In some cases, the kit further comprises amplification reagents. In some cases, the kit further comprises a biological sample. In some cases, the biological sample may comprise a tissue. In some cases, the biological sample may be fixed and/or permeabilized. In some cases, the biological sample may be loaded on the first substrate. In some cases, the kit further comprises fixing and/or permeabilizing reagents. In some cases, a bead of the plurality of beads may comprise at least 100,000 oligonucleotide molecules. In some cases, the at least 100,000 oligonucleotide molecules may comprise a barcode sequence of the barcode sequences that is common and unique to the bead amongst the plurality of beads. In some cases, an oligonucleotide molecule of the oligonucleotide molecules may comprises a capture sequence, wherein the capture sequence is configured to hybridize with a sequence of an analyte, or derivative thereof, of a biological sample. The capture sequence may be selected from, for example, a polyT sequence, a polyG sequence, a targeted mRNA sequence, a targeted gDNA sequence, a random n-mer sequence, and a probe sequence. The first and second substrate may be substantially identical in size, shape, and/or material.

In another example, a kit comprises a substrate comprising a plurality of beads immobilized to a plurality of individually addressable locations on the substrate, wherein the plurality of beads comprises oligonucleotide molecules coupled thereto, wherein the oligonucleotide molecules comprise barcode sequences, wherein the oligonucleotide molecules are releasable from the plurality of beads by one or more of the release mechanisms selected from the group consisting of: (a) providing an enzyme mix comprising an enzyme configured to cleave or digest a cleavage site, wherein the oligonucleotide molecules comprises the cleavage site; (b) providing biotin, wherein the oligonucleotide molecules are conjugated to a desthiobiotin moiety, wherein the bead comprises a streptavidin moiety bound to the desthiobiotin moiety; (c) providing an enzyme mix comprising an enzyme configured to cleave or digest a cleavage site and UV light, wherein the oligonucleotide molecules comprises azobenzene and the cleavage site; and (d) providing UV light, wherein the oligonucleotide molecules are conjugated azobenzene, wherein the plurality of beads comprises an alpha-cyclodextrine (a-CD) moiety bound to the azobenzene. In some cases, the kit may further comprise indexed data comprising identities and locations of the barcode sequences on the substrate. In some cases, the kit may further comprise a second substrate comprising a second plurality of individually addressable locations configured to immobilize a second plurality of beads. In some cases, the kit may further comprise the second plurality of beads. In some cases, the kit further comprises a reagent configured to release the oligonucleotide molecules from the plurality of beads. The reagent can comprise one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene. In some cases, the kit further comprises sequencing reagents, such as single-base nucleotide mixtures (e.g., A, C, G, T or U) or multi-base nucleotide mixtures (e.g., A&C, A&T, A&C&G, etc.). A single-base or multi-base nucleotide mixture may comprise a mixture of labeled and unlabeled nucleotides. A single-base or multi-base nucleotide mixture may comprise non-terminated nucleotides, in some cases comprising only non-terminated nucleotides (vs terminated nucleotides). In some cases, the kit further comprises amplification reagents. In some cases, the kit further comprises a biological sample. In some cases, the biological sample may comprise a tissue. In some cases, the biological sample may be fixed and/or permeabilized. In some cases, the biological sample may be loaded on the first substrate. In some cases, the kit further comprises fixing and/or permeabilizing reagents. In some cases, a bead of the plurality of beads may comprise at least 100,000 oligonucleotide molecules. In some cases, the at least 100,000 oligonucleotide molecules may comprise a barcode sequence of the barcode sequences that is common and unique to the bead amongst the plurality of beads. In some cases, an oligonucleotide molecule of the oligonucleotide molecules may comprises a capture sequence, wherein the capture sequence is configured hybridize with a sequence of an analyte, or derivative thereof, of a biological sample. The capture sequence may be selected from, for example, a polyT sequence, a polyG sequence, a targeted mRNA sequence, a targeted gDNA sequence, a random n-mer sequence, and a probe sequence. The first and second substrate may be substantially identical in size, shape, and/or material.

In one example, a system comprises a sequencing platform configured to (i) address individually addressable locations of substrates and (ii) rotate the substrates during dispensing of sequencing reagents to the substrates or during imaging of the substrates or during both. The sequencing platform may be any sequencing platform described herein. The system may further comprise any kit and/or reagent described herein (e.g., substrate, beads, indexed data, reagent configured to release oligonucleotide molecules from a plurality of beads, sequencing reagent, amplification reagent, fixing and/or permeabilizing reagent, etc.). In some cases, the system may comprise a light source configured to provide light at desired frequencies (e.g., UV light, fluorescent light, etc.).

### Computer Systems

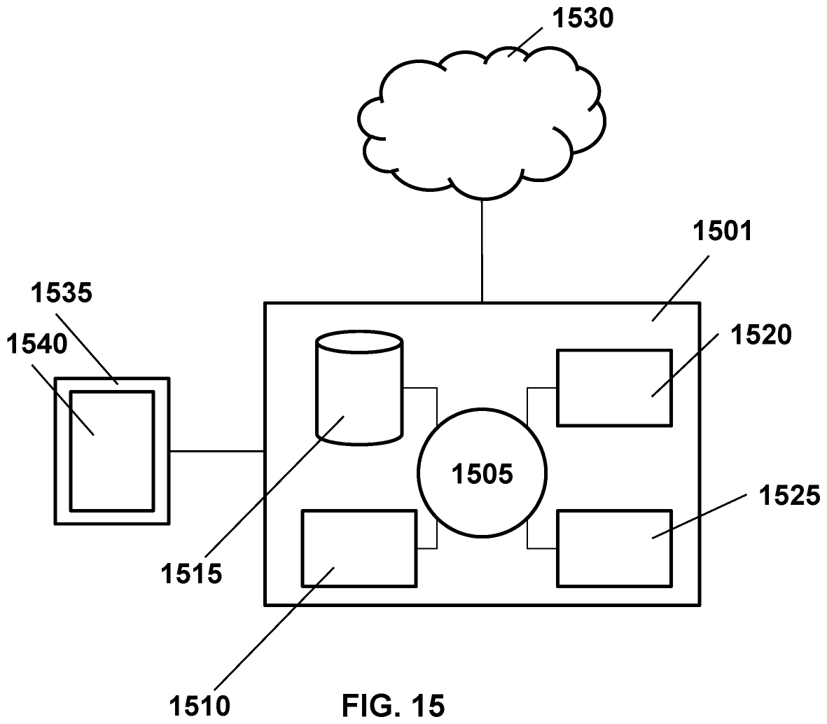

The present disclosure provides computer control systems that are programmed to implement methods of the disclosure. FIG. 15 shows a computer system 1501 that is programmed or otherwise configured to implement methods of the disclosure, such as to control the systems described herein (e.g., reagent dispensing, detecting, etc.) and collect, receive, and/or analyze sequencing information to conduct spatial analysis of a sample. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an isolated or substantially isolated internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1435 that comprises a user interface (UI) 1540 for providing, for example, provide examples: e.g., results of a nucleic acid sequence (e.g., sequence reads), indexing data, and spatial data. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405. The algorithm can, for example, spatially resolve a plurality of analyte sequences using sequencing information.

### Example 1. Imaging of Sequencing of a Nucleic Acid Molecule

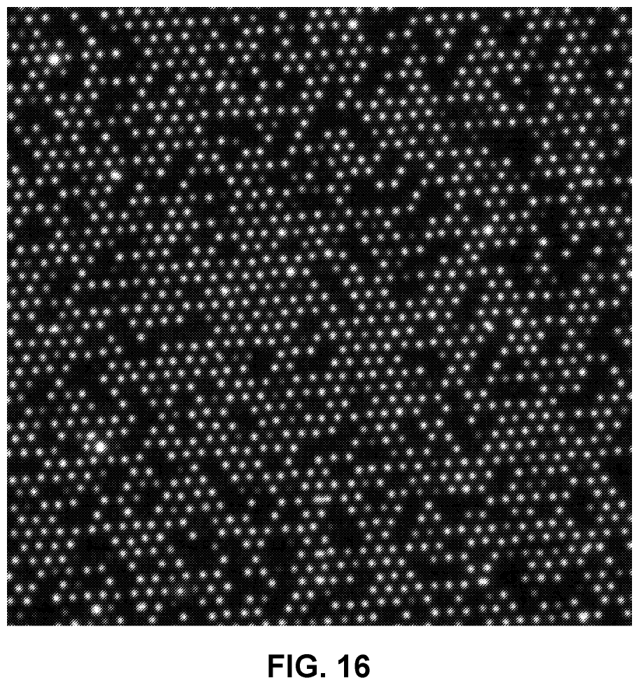

FIG. 16 shows an example of an image generated by imaging a substrate with an analyte immobilized thereto using the substrate-based sample processing systems described herein. A substrate comprising a substantially planar array has immobilized thereto the biological analyte, e.g., nucleic acid molecules. The substantially planar array comprises a plurality of individually addressable locations, and the plurality of the individually addressable locations comprises a biological analyte, e.g., one or more nucleic acid molecules. The individually addressable locations may be randomly arranged or arranged in an ordered pattern. The biological analyte may be attached to a bead, which is immobilized to the array. A single bead may comprise a plurality of analytes. A bead may be associated with an individually addressable location. A plurality of fluorescent probes (e.g., a plurality of fluorescently-labeled, A, T, C, or G base nucleotides) is dispensed onto the substrate. In some embodiments, the substrate is configured to rotate with respect to a central axis; a fluid flow unit comprising a fluid channel is configured to dispense a solution comprising a plurality of probes to the array, wherein during rotation of the substrate, the solution is directed centrifugally along a direction away from the central axis and brought in contact with the biological analyte. In other embodiments, the substrate is not rotated. The substrate is then subjected to conditions sufficient to conduct a reaction between at least one probe of the plurality of probes and the biological analyte, to couple the at least one probe to the biological analyte. The uncoupled probes are washed away. The coupling of the at least one probe to the biological analyte is detected using photometry, which comprises imaging at least a part of the substrate (e.g., via scanning or fixed field imaging) and measuring the signal of each individually addressable location. Nucleic acid molecules comprising a nucleotide complementary to the fluorescent probes are fluorescent in an individually addressable location. The operations may then be iterated, and signals from an image are collated with signals from prior images of the same substrate to generate traces of signals in time for each biological analyte in each individually addressable location. The sequence of the plurality of fluorescent probes is known for each iteration of the operations, generating a known sequence for the analyte in each of the individually addressable locations.

### Example 2. Sequencing of Nucleic Acid Molecules and Signal Processing

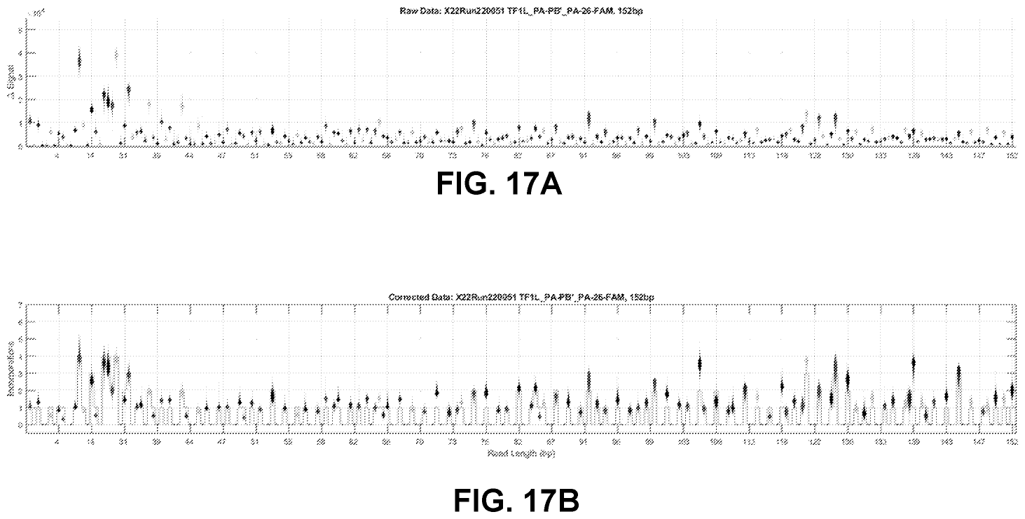

A substrate comprising a substantially planar array has immobilized thereto the biological analyte, e.g. nucleic acid molecules from E. coli. Sequencing by synthesis was performed using a flow-based chemistry. Imaging was performed, as described elsewhere herein. FIG. 17A shows the signal distributions for a set of several hundred colonies, each a replicate of a single synthetic monotemplate. The x-axis is labeled with the length of the sequencing after each cycle (e.g., each chemistry flow step). In FIG. 17B, the same data have been processed with a parametric model. The parametric model accounts for different template counts (amplitude) and background level for each colony. The signal is deconvolved with a model of lead and lag phasing and also accounts for global signal loss per cycle. In the example depicted here, the nominal phasing was 0.54% lag, 0.41% lead, and a signal loss of 0.45%. The residual systematic variation may be attributable to signal variation with sequence context can be further corrected using other algorithms (not shown).

### Example 3. Sequencing of Shotgun Library from E. coli

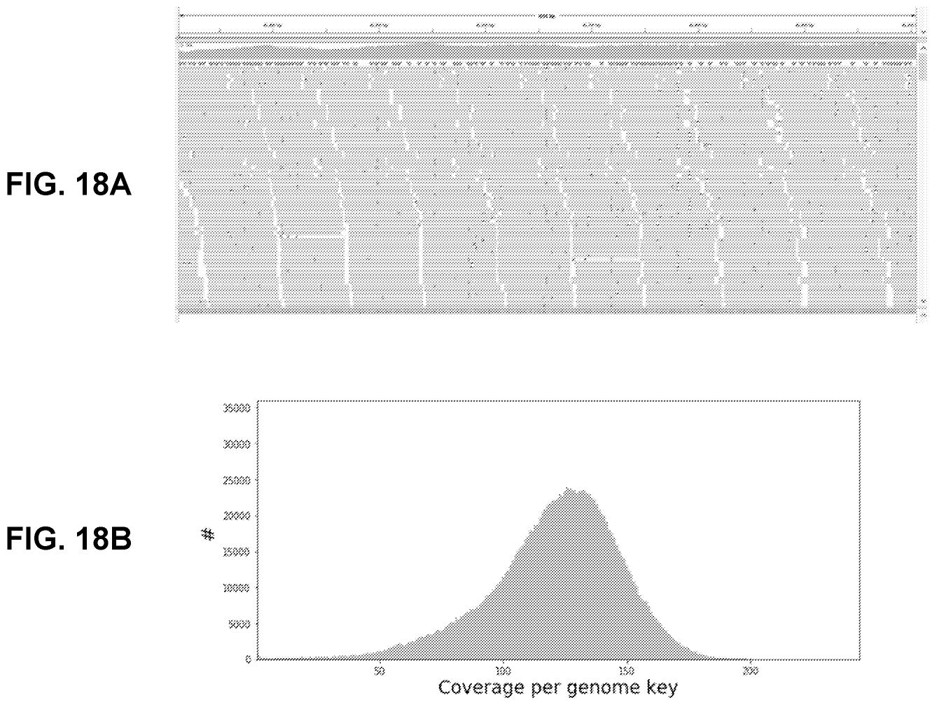

A substrate comprising a substantially planar array has immobilized thereto the biological analyte, e.g. nucleic acid molecules from E. coli. Sequencing by synthesis was performed using a flow-based chemistry. Imaging was performed, as described elsewhere herein. Images were then processed. FIG. 18A shows individual aligned reads for a segment of the E. coli reference genome. FIG. 18B shows a plot derived from the image processing of the aligned read depth for each position in the E. coli genome for a set of shotgun reads. The x-axis shows the coverage level at each E. coli reference key position and the y-axis shows the frequency.

### Example 4: Sequencing Nucleic Acid Molecules Using Blocked or Terminated Nucleotides

Nucleic acid molecules may be sequenced using the methods and systems provided herein. A nucleic acid molecule may be immobilized to a substrate (e.g., directly or via a support such as a bead, which bead may comprise a plurality of nucleic acid molecules coupled thereto, such as a clonal population of nucleic acid molecules). The substrate (e.g., a substrate, as described herein) may comprise a substantially planar array, which substantially planar array may comprise a plurality of individually addressable locations (e.g., individually addressable locations, as described herein). The plurality of individually addressable locations may be randomly arranged or arranged in an ordered pattern. The nucleic acid molecule may be associated with an individually addressable location of the array. For example, a bead to which the nucleic acid molecule is coupled may be associated with an individually addressable location of the array. The nucleic acid molecule may be coupled to the array (e.g., via a support coupled to the substrate) via an oligonucleotide such as an adapter or primer molecule. The substrate may be configured to rotate with respect to a central axis; a fluid flow unit comprising a fluid channel configured to dispense a solution may be coupled to the substrate such that, during rotation of the substrate, the solution is directed centrifugally along a direction away from the central axis and brought in contact with the biological analyte (e.g., nucleic acid molecule). Alternatively, the substrate may not be rotated.

The nucleic acid molecule may comprise a double-stranded region, which double-stranded region may comprise an adapter sequence in a first strand and a sequence complementary to the adapter sequence in the second strand. The nucleic acid molecule may comprise a target sequence (e.g., a library insert sequence), which target sequence may be flanked by one or more adapter sequences and one or more other sequences, such as one or more barcode or identifier sequences, primer sequences, or other sequences. The nucleic acid molecule may derive from a sample, such as a sample comprising a biological fluid (e.g., blood or saliva). The nucleic acid molecule may comprise deoxyribonucleic acid or ribonucleic acid. For example, the nucleic acid molecule may comprise genomic DNA.

Sequencing of the nucleic acid molecule may proceed by providing a first nucleotide that is complementary to an available position of the nucleic acid molecule (as part of an extending primer molecule). The first nucleotide may comprise a blocking or terminating group, such as a reversible terminator. The blocking or terminating group (e.g., reversible terminator) may be coupled to the first nucleotide via a sugar moiety, such as to a 3′ position of the sugar moiety. The blocking or terminating group may comprise an azido moiety. For example, the blocking or terminating group may be a 3′-O-azidomethyl blocking group. Alternatively, the blocking or terminating group may be another group that does not significantly affect incorporation of subsequent nucleotides into a template, such as a small, stable group. The first nucleotide may be labeled (e.g., may be coupled to a fluorescent label). Alternatively, the first nucleotide may be unlabeled (e.g., may not be coupled to a fluorescent label). The first nucleotide may be provided in a first solution (e.g., a reaction mixture), which first solution may comprise one or more additional nucleotides. The first solution may be provided to the substrate via the fluid channel of the fluid flow unit coupled to the substrate (e.g., during rotation of the substrate or while the substrate is stationary). The first solution may comprise a plurality of identical nucleotides comprising the first nucleotide. Alternatively, the first solution may comprise a first plurality of identical nucleotides comprising the first nucleotide and a second plurality of identical nucleotides, where the first nucleotide and a second nucleotide of the second plurality of identical nucleotides may have different chemical structures. For example, the first nucleotide and second nucleotide may comprise different bases (e.g., canonical bases, such as A, G, C, and U/T), labels (e.g., fluorescent labels), linkers (e.g., linkers connecting labels to bases, sugars, or phosphate moieties of a nucleotide), sugar moieties (e.g., sugar moieties comprising or not comprising blocking or terminating groups), or a combination thereof. In an example, the first solution comprises a first plurality of identical nucleotides comprising the first nucleotide, a second plurality of identical nucleotides, a third plurality of identical nucleotides, and a fourth plurality of identical nucleotides, wherein each plurality of identical nucleotides comprises bases of a different canonical type (e.g., A, G, C, and U/T). Each nucleotide of each plurality of identical nucleotides may comprise a blocking or terminating moiety, which blocking or terminating moiety may be the same or different for different types of nucleotides. Each nucleotide of each plurality of identical nucleotides may be unlabeled. Alternatively, all or a portion of each nucleotide of a given plurality of identical nucleotides may be labeled (e.g., with fluorescent labels). For example, all or a portion of each nucleotide of each plurality of identical nucleotides may be labeled. The first solution may comprise other reagents for performing a reaction, such as a buffer, cations, an enzyme (e.g., a polymerase enzyme), or other reagents.

The nucleic acid molecule coupled to the substantially planar array of the substrate and the first nucleotide may be subjected to conditions sufficient to incorporate the first nucleotide into an available position of the nucleic acid molecule (e.g., into a growing strand coupled to a nucleic acid strand comprising a target sequence). The blocking or terminating group of the first nucleotide may prevent incorporation of an additional nucleotide (e.g., of a same type, such as for a homopolymer sequence, or of a different type).

Incorporation of the first nucleotide into the nucleic acid molecule may be detected via imaging, such as by imaging a label coupled to the first nucleotide or a label of a reporter moiety. The array may be interrogated with a detector such an optical detector. Imaging may be performed during rotation of the substrate or while the substrate is stationary. Imaging may comprise scanning or fixed field imaging. For example, an optical detector may be translated and/or rotated relative to the substrate during imaging. Imaging may detect a signal (e.g., fluorescence emission) of a label (e.g., of the first nucleotide or of a reporter moiety coupled thereto). The signal may be indicative of the type of nucleotide incorporated into the nucleic acid molecule. Alternatively, the signal may be indicative of the type of reporter moiety coupled to the nucleic acid molecule, and thus of the type of nucleotide incorporated into the nucleic acid molecule.

After detection of the incorporation of the first nucleotide into the nucleic acid molecule, the process may be repeated using a second solution comprising a second nucleotide, etc., to determine a sequence of the nucleic acid molecule.

### Example 5: Sequencing Nucleic Acid Molecules Using Non-Terminated Nucleotides

Nucleic acid molecules may be sequenced using the methods and systems provided herein. A nucleic acid molecule may be immobilized to a substrate (e.g., directly or via a support such as a bead, which bead may comprise a plurality of nucleic acid molecules coupled thereto, such as a clonal population of nucleic acid molecules). The substrate (e.g., a substrate as described herein) may comprise a substantially planar array, which substantially planar array may comprise a plurality of individually addressable locations (e.g., individually addressable locations as described herein). The plurality of individually addressable locations may be randomly arranged or arranged in an ordered pattern. The nucleic acid molecule may be associated with an individually addressable location of the array. For example, a bead to which the nucleic acid molecule is coupled may be associated with an individually addressable location of the array. The nucleic acid molecule may be coupled to the array (e.g., via a support coupled to the substrate) via an oligonucleotide such as an adapter or primer molecule. The substrate may be configured to rotate with respect to a central axis; a fluid flow unit comprising a fluid channel configured to dispense a solution may be coupled to the substrate such that, during rotation of the substrate, the solution is directed centrifugally along a direction away from the central axis and brought in contact with the biological analyte (e.g., nucleic acid molecule). Alternatively, the substrate may not be rotated.

In some cases, the nucleic acid molecule may comprise a double-stranded region, which double-stranded region may comprise an adapter sequence in a first strand and a sequence complementary to the adapter sequence in the second strand. The nucleic acid molecule may comprise a target sequence (e.g., a library insert sequence), which target sequence may be flanked by one or more adapter sequences and one or more other sequences, such as one or more barcode or identifier sequences, primer sequences, or other sequences. The nucleic acid molecule may derive from a sample, such as a sample comprising a biological fluid (e.g., blood or saliva). The nucleic acid molecule may comprise deoxyribonucleic acid or ribonucleic acid. For example, the nucleic acid molecule may comprise genomic DNA.

Sequencing of the nucleic acid molecule may proceed by providing a first nucleotide that is complementary to an available position of the nucleic acid molecule (at an extending primer molecule hybridized to the nucleic acid molecule). The first nucleotide may be a non-terminated nucleotide (e.g., may not comprise a blocking or terminating group). The first nucleotide may be labeled (e.g., may be coupled to a fluorescent label). Alternatively, the first nucleotide may be unlabeled (e.g., may not be coupled to a fluorescent label). The first nucleotide may be provided in a first solution (e.g., a reaction mixture), which first solution may comprise one or more additional nucleotides. The first solution may be provided to the substrate via the fluid channel of the fluid flow unit coupled to the substrate (e.g., during rotation of the substrate or while the substrate is stationary). The first solution may comprise a plurality of identical nucleotides comprising the first nucleotide. Alternatively, the first solution may comprise a first plurality of identical nucleotides comprising the first nucleotide and a second plurality of identical nucleotides, where the first nucleotide and a second nucleotide of the second plurality of identical nucleotides may have different chemical structures. For example, the first nucleotide and second nucleotide may comprise different bases (e.g., canonical bases, such as A, G, C, and U/T), labels (e.g., fluorescent labels), linkers (e.g., linkers connecting labels to bases, sugars, or phosphate moieties of a nucleotide), sugar moieties, or a combination thereof. In an example, the first solution comprises a first plurality of identical nucleotides comprising the first nucleotide, a second plurality of identical nucleotides, a third plurality of identical nucleotides, and a fourth plurality of identical nucleotides, wherein each plurality of identical nucleotides comprises bases of a different canonical type (e.g., A, G, C, and U/T). Each nucleotide of each plurality of identical nucleotides may be unlabeled. Alternatively, all or a portion of each nucleotide of a given plurality of identical nucleotides may be labeled (e.g., with fluorescent labels). For example, all or a portion of each nucleotide of each plurality of identical nucleotides may be labeled. In an example, the first solution may comprise a plurality of nucleotides comprising the first nucleotide, in which each nucleotide includes the same canonical base. The plurality of nucleotides may comprise a plurality of labeled nucleotides and a plurality of unlabeled nucleotides. In some cases, at most 20% of the nucleotides, at most 10%, or at most 5% of the nucleotides in the solution are labeled and the remaining unlabeled. In other cases, at least 20%, at least 50%, at least 70%, or at least 90% of the nucleotides of the plurality of nucleotides of the first solution may be labeled nucleotides. Any % of the nucleotides of the plurality of nucleotides may be labeled nucleotides. The first solution may comprise other reagents for performing a reaction, such as a buffer, cations, an enzyme (e.g., a polymerase enzyme), or other reagents.

The nucleic acid molecule coupled to the substantially planar array of the substrate and the first nucleotide may be subjected to conditions sufficient to incorporate the first nucleotide into an available position of the nucleic acid molecule (e.g., into a growing strand coupled to a nucleic acid strand comprising a target sequence). The absence of a blocking or terminating group may facilitate incorporation of an additional nucleotide (e.g., of a same type, such as for a homopolymer sequence, or of a different type) in a position adjacent to that into which the first nucleotide is incorporated.

Where the first solution includes nucleotides comprising the same base (e.g., canonical base, such as A, G, C, and U/T), detection of incorporation of the first nucleotide and, in some cases (e.g., where the target sequence comprises a homopolymer sequence), one or more additional nucleotides may be detected by imaging a label coupled to the first nucleotide and/or the one or more additional nucleotides, or by detecting a label of a reporter moiety provided to the nucleic acid molecule (e.g., a reporter moiety configured to specifically bind to a nucleotide of a given type). A label coupled to an incorporated nucleotide may be removed (e.g., by contacting the incorporated nucleotide with a cleaving reagent) subsequent to detection, such as prior to contacting the nucleic acid molecule with a second solution comprising a second nucleotide. A label coupled to a reporter moiety may be similarly removed. Alternatively, a sequencing process may proceed without cleaving a label associated with a nucleotide incorporated into the nucleic acid molecule.

Where the first solution includes nucleotides comprising different bases, detection of incorporation of the first nucleotide and, in some cases (e.g., where the target sequence comprises a homopolymer sequence), one or more additional nucleotides may be detected by imaging a label coupled to the first nucleotide and/or the one or more additional nucleotides, which label may be different from other labels coupled to nucleotides comprising bases of different types. For example, the first nucleotide may comprise a label of a first type and a second nucleotide included in the first solution may comprise a label of a second type. The different labels may provide different signals, such as different fluorescence signatures, such that detection of the fluorescence signature of the label coupled to the first nucleotide indicates incorporation of the first nucleotide, rather than the second nucleotide. Alternatively, a labeled reporter moiety may be used to detect incorporation of a nucleotide of a given type.

The array may be interrogated with a detector such an optical detector. Imaging may be performed during rotation of the substrate or while the substrate is stationary. Imaging may comprise scanning or fixed field imaging. For example, an optical detector may be translated and/or rotated relative to the substrate during imaging. Imaging may detect a signal (e.g., fluorescence emission) of a label (e.g., of the first nucleotide or of a reporter moiety coupled thereto). The signal may be indicative of the type of nucleotide incorporated into the nucleic acid molecule. Alternatively, the signal may be indicative of the type of reporter moiety coupled to the nucleic acid molecule, and thus of the type of nucleotide incorporated into the nucleic acid molecule.

Additional details of such methods are described in the Examples below. After detection of the incorporation of the first nucleotide into the nucleic acid molecule, the process may be repeated using a second solution comprising an additional nucleotide, etc., to determine a sequence of the nucleic acid molecule.

### Example 6: Detecting Nucleotide Incorporations Using Reporter Moieties

As described in the preceding Examples, detection of incorporation of a nucleotide into a nucleic acid molecule may comprise detection of a label coupled to a nucleotide. Detection of incorporation of a nucleotide may alternatively comprise detection of a label coupled to a reporter moiety.

A labeled (e.g., fluorescently labeled) reporter moiety may be provided to a nucleic acid molecule coupled to a substantially planar array of a substrate (e.g., via a particle). A first nucleotide may be incorporated into the nucleic acid molecule (e.g., as described in the preceding Examples). The first nucleotide may comprise a blocking or terminating moiety. Alternatively, the first nucleotide may be a non-terminated nucleotide. The first reporter moiety may be provided in a first solution (e.g., a first solution providing the first nucleotide to the nucleic acid molecule for incorporation therein) or in a second solution that is provided to the nucleic acid molecule (e.g., after removal of the first solution via centrifugal action and optional application of a washing solution). The second solution may be provided during rotation of the substrate or while the substrate is stationary. The first reporter moiety may comprise an antibody. The first reporter moiety may comprise a fluorescent label. The first reporter moiety may be configured to bind to a nucleotide incorporated into a nucleic acid molecule. For example, the first reporter moiety may be base-specific. The first reporter moiety may be configured to bind to a nucleotide comprising a blocking or terminating group. For example, the first reporter moiety may be a base-specific, 3′ block-dependent first reporter moiety, such as a base-specific, 3′ block-dependent fluorescently labeled antibody. The first reporter moiety may be configured to bind to the first nucleotide. The first reporter moiety may be configured to not bind to a nucleotide of a type other than that of the first nucleotide. The solution comprising the first reporter moiety (e.g., the second solution) may comprise a plurality of identical first reporter moieties comprising the first reporter moiety. The solution comprising the first reporter moiety may also comprise a plurality of identical second reporter moieties specific to a second nucleotide type (e.g., of a second plurality of identical nucleotides), a plurality of identical third reporter moieties specific to a third nucleotide type (e.g., of a third plurality of identical nucleotides), and a plurality of identical fourth reporter moieties specific to a fourth nucleotide type (e.g., of a fourth plurality of identical nucleotides). Each plurality of identical reporter moieties may comprise a label of a different type. The first reporter moiety and the nucleic acid molecule may be subjected to conditions sufficient to bind the first reporter moiety and the first nucleotide incorporated into the nucleic acid molecule. Unbound reporter moieties may be removed (e.g., via removal of the second solution via centrifugal action and optional application of a washing solution). The array may be interrogated with a detector such an optical detector. Imaging may be performed during rotation of the substrate or while the substrate is stationary. Imaging may comprise scanning or fixed field imaging. For example, an optical detector may be translated and/or rotated relative to the substrate during imaging. Imaging may detect a signal (e.g., fluorescence emission) of a label of the first reporter moiety. The signal may be indicative of the type of reporter moiety coupled to the nucleic acid molecule, and thus of the type of nucleotide incorporated into the nucleic acid molecule.

Subsequent to imaging, the nucleic acid molecule coupled to the array may be subjected to conditions sufficient to remove the first reporter moiety coupled to the first nucleotide. For example, a washing solution may be provided that may comprise a reagent configured to cleave the blocking or terminating group from the first nucleotide and remove the first reporter moiety. Subsequent to the cleaving/washing process, the first nucleotide may no longer comprise a blocking or terminating group, such that the incorporation and detection process may be repeated one or more times. In this manner, a sequence of the nucleic acid molecule coupled to the array may be determined. This process may be used to identify sequences of a plurality of nucleic acid molecules, such as one or more clonal populations of nucleic acid molecules coupled to the array. For example, this process may be used to identify sequences of multiple different clonal populations of nucleic acid molecules coupled to a plurality of beads coupled to a plurality of individually addressable locations of the substantially planar array of the substrate.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

1. A method, comprising: (a) immobilizing a plurality of beads to individually addressable locations of a first substrate to provide a barcoded substrate, wherein the plurality of beads comprises oligonucleotide molecules coupled thereto, wherein the oligonucleotide molecules comprise barcode sequences; (b) sequencing the oligonucleotide molecules on the barcoded substrate to generate indexed data that indexes the individually addressable locations of the first substrate with the barcode sequences of the oligonucleotide molecules, to provide an indexed, barcoded substrate, wherein the sequencing comprises rotating the barcoded substrate during dispensing of sequencing reagents to the barcoded substrate or during imaging of the barcoded substrate; (c) loading a biological sample to the indexed, barcoded substrate to tag analytes in the biological sample using the oligonucleotide molecules, to generate spatially tagged analytes, wherein the spatially tagged analytes comprise the barcode sequences or derivatives thereof; (d) immobilizing the spatially tagged analytes, or derivatives thereof, on a second substrate; (e) sequencing the spatially tagged analytes, or derivatives thereof, on the second substrate to generate sequencing data, wherein the sequencing comprises rotating the second substrate during dispensing of sequencing reagents to the second substrate or during imaging of the second substrate; and (f) generating spatial data of the analytes of the biological sample using the sequencing data and the indexed data. 2. The method of embodiment 1, wherein a bead of the plurality of beads comprises at least 100,000 oligonucleotide molecules. 3. The method of embodiment 2, wherein the at least 100,000 oligonucleotide molecules comprise a barcode sequence of the barcode sequences that is common and unique to the bead amongst the plurality of beads. 4. The method of any one of embodiments 1-3, wherein an oligonucleotide molecule of the oligonucleotide molecules comprises a capture sequence, wherein the capture sequence hybridizes with a sequence of an analyte, or derivative thereof, of the analytes. 5. The method of embodiment 4, wherein the capture sequence is selected from the group consisting of: a polyT sequence, a polyG sequence, a targeted mRNA sequence, a targeted gDNA sequence, a random n-mer sequence, and a probe sequence. 6. The method of any one of embodiments 1-5, wherein the biological sample comprises a tissue. 7. The method of any one of embodiments 1-6, wherein the analytes comprise mRNA. 8. The method of any one of embodiments 1-6, wherein the analytes comprise DNA. 9. The method of any one of embodiments 1-6, wherein the analytes comprise proteins. 10. The method of any one of embodiments 1-9, wherein the first substrate comprises at least 1,000,000 individually addressable locations. 11. The method of embodiment 10, wherein the first substrate comprises at least 1,000,000,000 individually addressable locations. 12. The method of any one of embodiments 1-11, wherein the plurality of beads is immobilized to the individually addressable locations via electrostatic interactions. 13. The method of any one of embodiments 1-12, wherein the spatially tagged analytes, or derivatives thereof, are coupled to a second plurality of beads, which second plurality of beads are immobilized to second individually addressable locations of the second substrate. 14. The method of embodiment 13, wherein the second substrate comprises at least 1,000,000 second individually addressable locations. 15. The method of embodiment 14, wherein the second substrate comprises at least 1,000,000,000 second individually addressable locations. 16. The method of any one of embodiments 13-15, wherein the second plurality of beads is immobilized to the second individually addressable locations via electrostatic interactions. 17. The method of any one of embodiments 13-16, wherein a second bead of the second plurality of beads comprises a colony of a spatially tagged analyte, or derivative thereof, of the spatially tagged analytes, or derivatives thereof. 18. The method of embodiment 17, wherein the second bead comprises at least 100,000 copies of the spatially tagged analyte, or derivative thereof. 19. The method of any one of embodiments 1-18, wherein the sequencing in (b) comprises sequencing by synthesis. 20. The method of embodiment 19, wherein the sequencing by synthesis comprises providing distinct nucleotide flows to the first substrate, wherein a distinct nucleotide flow of the distinct nucleotide flows comprises a single-base nucleotide mixture. 21. The method of embodiment 20, wherein the single-base nucleotide mixture comprises a mixture of labeled nucleotides and non-labeled nucleotides of a single base. 22. The method of any one of embodiments 20-21, wherein the single-base nucleotide mixture comprises non-terminated nucleotides. 23. The method of any one of embodiments 1-22, wherein the sequencing in (b) comprises rotating the barcoded substrate during dispensing of sequencing reagents. 24. The method of any one of embodiments 1-23, wherein the sequencing in (b) comprises rotating the barcoded substrate during imaging of the barcoded substrate. 25. The method of any one of embodiments 1-24, wherein the sequencing in (b) comprises rotating the barcoded substrate during dispensing of sequencing reagents and rotating the barcoded substrate during imaging of the barcoded substrate. 26. The method of any one of embodiments 1-25, wherein the sequencing in (e) comprises sequencing by synthesis. 27. The method of embodiment 26, wherein the sequencing by synthesis comprises providing distinct nucleotide flows to the second substrate, wherein a distinct nucleotide flow of the distinct nucleotide flows comprises a single-base nucleotide mixture. 28. The method of embodiment 27, wherein the single-base nucleotide mixture comprises a mixture of labeled nucleotides and non-labeled nucleotides of a single base. 29. The method of any one of embodiments 27-28, wherein the single-base nucleotide mixture comprises non-terminated nucleotides. 30. The method of any one of embodiments 1-29, wherein the sequencing in (e) comprises rotating the second substrate during dispensing of sequencing reagents. 31. The method of any one of embodiments 1-30, wherein the sequencing in (e) comprises rotating the second substrate during imaging of the second substrate. 32. The method of any one of embodiments 1-31, wherein the sequencing in (e) comprises rotating the second substrate during dispensing of sequencing reagents and rotating the second substrate during imaging of the second substrate. 33. The method of any one of embodiments 1-32, wherein the first substrate or the second substrate is substantially planar. 34. The method of any one of embodiments 1-33, further comprising releasing the oligonucleotide molecules from the plurality of beads. 35. The method of embodiment 34, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene. 36. The method of any one of embodiments 1-35, further comprising incubating the biological sample on the indexed, barcoded substrate. 37. The method of any one of embodiments 1-36, further comprising fixing the biological sample. 38. The method of any one of embodiments 1-37, further comprising permeabilizing the biological sample.

39. A method, comprising: (a) providing (1) a first substrate comprising a plurality of beads immobilized thereto, wherein the plurality of beads comprises oligonucleotide molecules coupled thereto, wherein the oligonucleotide molecules comprise barcode sequences, and (2) indexed data comprising identities and locations of the barcode sequences on the first substrate; (b) loading a biological sample to the first substrate to tag analytes in the biological sample using the oligonucleotide molecules, to generate spatially tagged analytes, wherein the spatially tagged analytes comprise the barcode sequences or derivatives thereof; (c) immobilizing the spatially tagged analytes, or derivatives thereof, on a second substrate; (d) sequencing the spatially tagged analytes, or derivatives thereof, on the second substrate to generate sequencing data, wherein the sequencing comprises rotating the second substrate during dispensing of sequencing reagents to the second substrate or during imaging of the second substrate; and (e) generating spatial data of the analytes of the biological sample using the sequencing data and the indexed data. 40. The method of embodiment 39, wherein a bead of the plurality of beads comprises at least 100,000 oligonucleotide molecules. 41. The method of embodiment 40, wherein the at least 100,000 oligonucleotide molecules comprise a barcode sequence of the barcode sequences that is common and unique to the bead amongst the plurality of beads. 42. The method of any one of embodiments 39-41, wherein an oligonucleotide molecule of the oligonucleotide molecules comprises a capture sequence, wherein the capture sequence hybridizes with a sequence of an analyte, or derivative thereof, of the analytes. 43. The method of embodiment 42, wherein the capture sequence is selected from the group consisting of: a polyT sequence, a polyG sequence, a targeted mRNA sequence, a targeted gDNA sequence, a random n-mer sequence, and a probe sequence. 44. The method of any one of embodiments 39-43, wherein the biological sample comprises a tissue. 45. The method of any one of embodiments 39-44, wherein the analytes comprise mRNA. 46. The method of any one of embodiments 39-44, wherein the analytes comprise DNA. 47. The method of any one of embodiments 39-44, wherein the analytes comprise proteins. 48. The method of any one of embodiments 39-47, wherein the first substrate comprises at least 1,000,000 individually addressable locations, wherein the plurality of beads are immobilized to the at least 1,000,000 individually addressable locations. 49. The method of embodiment 48, wherein the first substrate comprises at least 1,000,000,000 individually addressable locations. 50. The method of any one of embodiments 48-49, wherein the plurality of beads is immobilized to the at least 1,000,000 individually addressable locations via electrostatic interactions. 51. The method of any one of embodiments 39-50, wherein the spatially tagged analytes, or derivatives thereof, are coupled to a second plurality of beads, which second plurality of beads are immobilized to second individually addressable locations of the second substrate. 52. The method of embodiment 51, wherein the second substrate comprises at least 1,000,000 second individually addressable locations. 53. The method of embodiment 52, wherein the second substrate comprises at least 1,000,000,000 second individually addressable locations. 54. The method of any one of embodiments 51-53, wherein the second plurality of beads is immobilized to the second individually addressable locations via electrostatic interactions. 55. The method of any one of embodiments 51-54, wherein a second bead of the second plurality of beads comprises a colony of a spatially tagged analyte, or derivative thereof, of the spatially tagged analytes, or derivatives thereof. 56. The method of embodiment 55, wherein the second bead comprises at least 100,000 copies of the spatially tagged analyte, or derivative thereof. 57. The method of any one of embodiments 39-56, wherein the sequencing in (d) comprises sequencing by synthesis. 58. The method of embodiment 57, wherein the sequencing by synthesis comprises providing distinct nucleotide flows to the second substrate, wherein a distinct nucleotide flow of the distinct nucleotide flows comprises a single-base nucleotide mixture. 59. The method of embodiment 58, wherein the single-base nucleotide mixture comprises a mixture of labeled nucleotides and non-labeled nucleotides of a single base. 60. The method of any one of embodiments 58-59, wherein the single-base nucleotide mixture comprises non-terminated nucleotides. 61. The method of any one of embodiments 39-60, wherein the sequencing in (d) comprises rotating the second substrate during dispensing of sequencing reagents. 62. The method of any one of embodiments 39-61, wherein the sequencing in (d) comprises rotating the second substrate during imaging of the second substrate. 63. The method of any one of embodiments 39-62, wherein the sequencing in (d) comprises rotating the second substrate during dispensing of sequencing reagents and rotating the second substrate during imaging of the second substrate. 64. The method of any one of embodiments 39-63, wherein the first substrate or the second substrate is substantially planar. 65. The method of any one of embodiments 39-64, further comprising releasing the oligonucleotide molecules from the plurality of beads. 66. The method of embodiment 65, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene. 67. The method of any one of embodiments 39-67, further comprising incubating the biological sample on the first substrate. 68. The method of any one of embodiments 39-68, further comprising fixing the biological sample. 69. The method of any one of embodiments 39-69, further comprising permeabilizing the biological sample.

70. A method, comprising: (a) sequencing a first substrate comprising a plurality of beads immobilized thereto, wherein the plurality of beads comprises oligonucleotide molecules coupled thereto, wherein the oligonucleotide molecules comprise barcode sequences, to generate indexed data comprising identities and locations of the barcode sequences on the first substrate; (b) loading a biological sample to the first substrate to tag analytes in the biological sample using the oligonucleotide molecules, to generate spatially tagged analytes, wherein the spatially tagged analytes comprise the barcode sequences or derivatives thereof; (c) immobilizing the spatially tagged analytes, or derivatives thereof, on a second substrate; and (d) sequencing the second substrate to generate sequencing data, wherein the first substrate or the second substrate or both are rotated during the sequencing in (a) or (d), respectively. 71. The method of embodiment 70, wherein a bead of the plurality of beads comprises at least 100,000 oligonucleotide molecules. 72. The method of embodiment 71, wherein the at least 100,000 oligonucleotide molecules comprise a barcode sequence of the barcode sequences that is common and unique to the bead amongst the plurality of beads. 73. The method of any one of embodiments 70-72, wherein an oligonucleotide molecule of the oligonucleotide molecules comprises a capture sequence, wherein the capture sequence hybridizes with a sequence of an analyte, or derivative thereof, of the analytes. 74. The method of embodiment 73, wherein the capture sequence is selected from the group consisting of: a polyT sequence, a polyG sequence, a targeted mRNA sequence, a targeted gDNA sequence, a random n-mer sequence, and a probe sequence. 75. The method of any one of embodiments 70-74, wherein the biological sample comprises a tissue. 76. The method of any one of embodiments 70-75, wherein the analytes comprise mRNA. 77. The method of any one of embodiments 70-75, wherein the analytes comprise DNA. 78. The method of any one of embodiments 70-75, wherein the analytes comprise proteins. 79. The method of any one of embodiments 70-78, wherein the first substrate comprises at least 1,000,000 individually addressable locations immobilizing the plurality of beads. 80. The method of embodiment 79, wherein the first substrate comprises at least 1,000,000,000 individually addressable locations immobilizing the plurality of beads. 81. The method of embodiment 80, wherein the plurality of beads is immobilized to the at least 1,000,000 individually addressable locations via electrostatic interactions. 82. The method of any one of embodiments 70-81, wherein the spatially tagged analytes, or derivatives thereof, are coupled to a second plurality of beads, which second plurality of beads are immobilized to second individually addressable locations of the second substrate. 83. The method of embodiment 82, wherein the second substrate comprises at least 1,000,000 second individually addressable locations. 84. The method of embodiment 83, wherein the second substrate comprises at least 1,000,000,000 second individually addressable locations. 85. The method of any one of embodiments 82-84, wherein the second plurality of beads is immobilized to the second individually addressable locations via electrostatic interactions. 86. The method of any one of embodiments 82-85, wherein a second bead of the second plurality of beads comprises a colony of a spatially tagged analyte, or derivative thereof, of the spatially tagged analytes, or derivatives thereof. 87. The method of embodiment 86, wherein the second bead comprises at least 100,000 copies of the spatially tagged analyte, or derivative thereof. 88. The method of any one of embodiments 70-87, wherein the sequencing in (a) comprises sequencing by synthesis. 89. The method of embodiment 88, wherein the sequencing by synthesis comprises providing distinct nucleotide flows to the first substrate, wherein a distinct nucleotide flow of the distinct nucleotide flows comprises a single-base nucleotide mixture. 90. The method of embodiment 89, wherein the single-base nucleotide mixture comprises a mixture of labeled nucleotides and non-labeled nucleotides of a single base. 91. The method of any one of embodiments 89-90, wherein the single-base nucleotide mixture comprises non-terminated nucleotides. 92. The method of any one of embodiments 70-91, wherein the sequencing in (a) comprises rotating the barcoded substrate during dispensing of sequencing reagents. 93. The method of any one of embodiments 70-92, wherein the sequencing in (a) comprises rotating the barcoded substrate during imaging of the barcoded substrate. 94. The method of any one of embodiments 70-93, wherein the sequencing in (a) comprises rotating the barcoded substrate during dispensing of sequencing reagents and rotating the barcoded substrate during imaging of the barcoded substrate. 95. The method of any one of embodiments 70-94, wherein the sequencing in (d) comprises sequencing by synthesis. 96. The method of embodiment 95, wherein the sequencing by synthesis comprises providing distinct nucleotide flows to the second substrate, wherein a distinct nucleotide flow of the distinct nucleotide flows comprises a single-base nucleotide mixture. 97. The method of embodiment 96, wherein the single-base nucleotide mixture comprises a mixture of labeled nucleotides and non-labeled nucleotides of a single base. 98. The method of any one of embodiments 96-97, wherein the single-base nucleotide mixture comprises non-terminated nucleotides. 99. The method of any one of embodiments 70-98, wherein the sequencing in (d) comprises rotating the second substrate during dispensing of sequencing reagents. 100. The method of any one of embodiments 70-99, wherein the sequencing in (d) comprises rotating the second substrate during imaging of the second substrate. 101. The method of any one of embodiments 70-100, wherein the sequencing in (d) comprises rotating the second substrate during dispensing of sequencing reagents and rotating the second substrate during imaging of the second substrate. 102. The method of any one of embodiments 70-101, wherein the first substrate or the second substrate is substantially planar. 103. The method of any one of embodiments 70-102, further comprising releasing the oligonucleotide molecules from the plurality of beads. 104. The method of embodiment 103, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene. 105. The method of any one of embodiments 70-104, further comprising incubating the biological sample on the first substrate. 106. The method of any one of embodiments 70-105, further comprising fixing the biological sample. 107. The method of any one of embodiments 70-106, further comprising permeabilizing the biological sample. 108. The method of any one of embodiments 70-107, further comprising generating spatial data of the analytes in the biological sample using at least the indexed data and the sequencing data.

109. A system, comprising: a sequencing platform configured to (i) address individually addressable locations of substrates and (ii) rotate the substrates during dispensing of sequencing reagents to the substrates or during imaging of the substrates or during both; and a first substrate comprising a plurality of beads immobilized to a plurality of individually addressable locations on the substrate, wherein the plurality of beads comprises oligonucleotide molecules coupled thereto, wherein the oligonucleotide molecules comprise barcode sequences. 110. The system of embodiment 109, further comprising indexed data comprising identities and locations of the barcode sequences on the first substrate. 111. The system of any one of embodiments 109-110, further comprising a second substrate comprising a second plurality of individually addressable locations configured to immobilize a second plurality of beads. 112. The system of embodiment 111, further comprising the second plurality of beads. 113. The system of any one of embodiments 109-112, further comprising a reagent configured to release the oligonucleotide molecules from the plurality of beads. 114. The system of embodiment 113, wherein the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene. 115. The system of any one of embodiments 109-114, further comprising sequencing reagents. 116. The system of embodiment 115, wherein the sequencing reagents comprise single-base nucleotide mixtures. 117. The system of embodiment 116, wherein a single-base nucleotide mixture of the single-base nucleotide mixtures comprises a mixture of labeled nucleotides and non-labeled nucleotides of a single base. 118. The system of any one of embodiments 116-117, wherein a single-base nucleotide mixture of the single-base nucleotide mixtures comprises non-terminated nucleotides. 119. The system of any one of embodiments 109-118, further comprising amplification reagents. 120. The system of any one of embodiments 109-119, further comprising a biological sample. 121. The system of embodiment 120, wherein the biological sample is a tissue. 122. The system of any one of embodiments 120-121, wherein the biological sample is fixed. 123. The system of any one of embodiments 120-122, wherein the biological sample is permeabilized. 124. The system of any one of embodiments 120-123, wherein the biological sample is loaded on the first substrate. 125. The system of any one of embodiments 109-124, wherein a bead of the plurality of beads comprises at least 100,000 oligonucleotide molecules. 126. The system of embodiment 125, wherein the at least 100,000 oligonucleotide molecules comprise a barcode sequence of the barcode sequences that is common and unique to the bead amongst the plurality of beads. 127. The system of any one of embodiments 109-126, wherein an oligonucleotide molecule of the oligonucleotide molecules comprises a capture sequence, wherein the capture sequence is configured to hybridize with a sequence of an analyte, or derivative thereof, of a biological sample. 128. The system of embodiment 127, wherein the capture sequence is selected from the group consisting of: a polyT sequence, a polyG sequence, a targeted mRNA sequence, a targeted gDNA sequence, a random n-mer sequence, and a probe sequence. 129. The system of any one of embodiments 109-128, wherein the first substrate comprises at least 1,000,000 individually addressable locations immobilizing the plurality of beads. 130. The system of embodiment 129, wherein the first substrate comprises at least 1,000,000,000 individually addressable locations immobilizing the plurality of beads. 131. The system of any one of embodiments 109-130, wherein the plurality of beads is immobilized to the individually addressable locations via electrostatic interactions. 132. The system of any one of embodiments 109-131, wherein the sequencing platform is configured to perform sequencing by synthesis on the substrates. 133. The system of any one of embodiments 109-132, wherein the first substrate is substantially planar.

134. A kit, comprising: a first substrate comprising a plurality of beads immobilized to a plurality of individually addressable locations on the substrate, wherein the plurality of beads comprises oligonucleotide molecules coupled thereto, wherein the oligonucleotide molecules comprise barcode sequences; indexed data comprising identities and locations of the barcode sequences on the first substrate; and a second substrate comprising a second plurality of individually addressable locations configured to immobilize a second plurality of beads. 135. The kit of embodiment 134, further comprising the second plurality of beads not immobilized to the second substrate. 136. The kit of any one of embodiments 134-135, further comprising a reagent configured to release the oligonucleotide molecules from the plurality of beads. 137. The kit of embodiment 136, wherein the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene. 138. The kit of any one of embodiments 134-137, further comprising sequencing reagents. 139. The kit of embodiment 138, wherein the sequencing reagents comprise single-base nucleotide mixtures. 140. The kit of embodiment 139, wherein a single-base nucleotide mixture of the single-base nucleotide mixtures comprises a mixture of labeled nucleotides and non-labeled nucleotides of a single base. 141. The kit of any one of embodiments 139-140, wherein a single-base nucleotide mixture of the single-base nucleotide mixtures comprises non-terminated nucleotides. 142. The kit of any one of embodiments 134-141, further comprising amplification reagents. 143. The kit of any one of embodiments 134-142, further comprising a biological sample. 144. The kit of embodiment 143, wherein the biological sample is a tissue. 145. The kit of any one of embodiments 143-144, wherein the biological sample is fixed. 146. The kit of any one of embodiments 143-145, wherein the biological sample is permeabilized. 147. The kit of any one of embodiments 139-146, wherein the biological sample is loaded on the first substrate. 148. The kit of any one of embodiments 134-147, wherein a bead of the plurality of beads comprises at least 100,000 oligonucleotide molecules. 149. The kit of embodiment 148, wherein the at least 100,000 oligonucleotide molecules comprise a barcode sequence of the barcode sequences that is common and unique to the bead amongst the plurality of beads. 150. The kit of any one of embodiments 134-149, wherein an oligonucleotide molecule of the oligonucleotide molecules comprises a capture sequence, wherein the capture sequence is configured to hybridize with a sequence of an analyte, or derivative thereof, of a biological sample. 151. The kit of embodiment 150, wherein the capture sequence is selected from the group consisting of: a polyT sequence, a polyG sequence, a targeted mRNA sequence, a targeted gDNA sequence, a random n-mer sequence, and a probe sequence. 152. The kit of any one of embodiments 134-151, wherein the first substrate comprises at least 1,000,000 individually addressable locations. 153. The kit of embodiment 152, wherein the first substrate comprises at least 1,000,000,000 individually addressable locations. 154. The kit of any one of embodiments 134-153, wherein the second substrate comprises at least 1,000,000 individually addressable locations. 155. The kit of embodiment 154, wherein the second substrate comprises at least 1,000,000,000 individually addressable locations. 156. The kit of any one of embodiments 134-155, wherein the plurality of beads is immobilized to the plurality of individually addressable locations via electrostatic interactions. 157. The kit of any one of embodiments 134-156, wherein the first substrate or the second substrate is substantially planar. 158. The kit of embodiment 157, wherein the first substrate and the second substrate are substantially planar. 159. The kit of any one of embodiments 134-158, wherein the first substrate and the second substrate are substantially identical in size.

160. A kit, comprising: a substrate comprising a plurality of beads immobilized to a plurality of individually addressable locations on the substrate, wherein the plurality of beads comprises oligonucleotide molecules coupled thereto, wherein the oligonucleotide molecules comprise barcode sequences, wherein the oligonucleotide molecules are releasable from the plurality of beads by one or more of the release mechanisms selected from the group consisting of: (a) providing an enzyme mix comprising an enzyme configured to cleave or digest a cleavage site, wherein the oligonucleotide molecules comprises the cleavage site; (b) providing biotin, wherein the oligonucleotide molecules are conjugated to a desthiobiotin moiety, wherein the bead comprises a streptavidin moiety bound to the desthiobiotin moiety; (c) providing an enzyme mix comprising an enzyme configured to cleave or digest a cleavage site and UV light, wherein the oligonucleotide molecules comprises azobenzene and the cleavage site; and (d) providing UV light, wherein the oligonucleotide molecules are conjugated azobenzene, wherein the plurality of beads comprises an alpha-cyclodextrine (a-CD) moiety bound to the azobenzene. 161. The kit of embodiment 160, further comprising indexed data comprising identities and locations of the barcode sequences on the substrate. 162. The kit of any one of embodiments 160-161, further comprising a second substrate comprising a second plurality of individually addressable locations configured to immobilize a second plurality of beads. 163. The kit of embodiment 162, further comprising the second plurality of beads. 164. The kit of any one of embodiments 160-163, further comprising a reagent configured to release the oligonucleotide molecules from the plurality of beads. 165. The kit of embodiment 164, wherein the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene. 166. The kit of any one of embodiments 160-165, further comprising sequencing reagents. 167. The kit of embodiment 166, wherein the sequencing reagents comprise single-base nucleotide mixtures. 168. The kit of embodiment 167, wherein a single-base nucleotide mixture of the single-base nucleotide mixtures comprises a mixture of labeled nucleotides and non-labeled nucleotides of a single base. 169. The kit of any one of embodiments 167-168, wherein a single-base nucleotide mixture of the single-base nucleotide mixtures comprises non-terminated nucleotides. 170. The kit of any one of embodiments 160-169, further comprising amplification reagents. 171. The kit of any one of embodiments 160-170, further comprising a biological sample. 172. The kit of embodiment 171, wherein the biological sample is a tissue. 173. The kit of any one of embodiments 171-172, wherein the biological sample is fixed. 174. The kit of any one of embodiments 171-173, wherein the biological sample is permeabilized. 175. The kit of any one of embodiments 171-174, wherein the biological sample is loaded on the first substrate. 176. The kit of any one of embodiments 160-175, wherein a bead of the plurality of beads comprises at least 100,000 oligonucleotide molecules. 177. The kit of embodiment 176, wherein the at least 100,000 oligonucleotide molecules comprise a barcode sequence of the barcode sequences that is common and unique to the bead amongst the plurality of beads. 178. The kit of any one of embodiments 160-177, wherein an oligonucleotide molecule of the oligonucleotide molecules comprises a capture sequence, wherein the capture sequence is configured to hybridize with a sequence of an analyte, or derivative thereof, of a biological sample. 179. The kit of embodiment 178, wherein the capture sequence is selected from the group consisting of: a polyT sequence, a polyG sequence, a targeted mRNA sequence, a targeted gDNA sequence, a random n-mer sequence, and a probe sequence. 180. The kit of any one of embodiments 160-179, wherein the substrate comprises at least 1,000,000 individually addressable locations. 181. The kit of embodiment 180, wherein the substrate comprises at least 1,000,000,000 individually addressable locations. 182. The kit of any one of embodiments 160-181, wherein the plurality of beads is immobilized to the individually addressable locations via electrostatic interactions. 183. The kit of any one of embodiments 160-182, wherein the substrate is substantially planar.

184. A method, comprising: (a) providing a substrate comprising a cell or tissue sample thereon; (b) subjecting said substrate to rotation to distribute one or more reagents to said cell or tissue sample; and (c) using a detector in optical communication with said cell or tissue sample on said substrate to detect an analyte of said cell or tissue sample.

185. A method, comprising: (a) providing a substrate comprising a cell or tissue sample thereon; (b) distributing one or more reagents to said cell or tissue sample; and (c) using a detector in optical communication with said cell or tissue sample on said substrate to detect an analyte of said cell or tissue sample, during rotation of said substrate.

186. The method of embodiment 184 or embodiment 185, wherein said analyte is within said cell or tissue sample. 187. The method of embodiment 186, wherein said analyte comprises a nucleic acid molecule, a polypeptide molecule, a protein molecule, a carbohydrate molecule, a lipid molecule, any derivative thereof, or any combination thereof. 188. The method of embodiment 187, wherein said analyte comprises said nucleic acid molecule, any derivative thereof, or any combination thereof. 189. The method of embodiment 188, wherein said nucleic acid molecule comprises ribonucleic acid (RNA) or derivative thereof. 190. The method of embodiment 189, wherein said RNA comprises a messenger RNA (mRNA). 191. The method of embodiment 188, wherein said nucleic acid molecule comprises deoxyribonucleic acid (DNA) or derivative thereof. 192. The method of embodiment 187, wherein said analyte comprises a polypeptide molecule or derivative thereof. 193. The method of embodiment 187, wherein said analyte comprises a protein molecule or derivative thereof. 194. The method of embodiment 187, wherein said analyte comprises a carbohydrate molecule. 195. The method of embodiment 194, wherein said carbohydrate comprises a monosaccharide, a polysaccharide or a lignin. 196. The method of embodiment 195, wherein said carbohydrate comprises a monosaccharide. 197. The method of embodiment 195, wherein said carbohydrate comprises a polysaccharide. 198. The method of embodiment 195, wherein said carbohydrate comprises a lignin. 199. The method of embodiment 184 or embodiment 185, wherein said one or more reagents comprises a probe configured to detect said analyte. 200. The method of embodiment 199, wherein said probe comprises one or more nucleotides. 201. The method of embodiment 199, wherein said probe comprises one or more nucleic acid molecules. 202. The method of embodiment 201, wherein said probe comprises a padlock probe. 203. The method of embodiment 201, wherein said probe comprises a barcode sequence. 204. The method of embodiment 201, wherein said one or more reagents comprises a second probe that pairs with said probe. 205. The method of embodiment 201, wherein said probe hybridizes to, or adjacent to, said analyte. 206. The method of embodiment 201, wherein said probe hybridizes to, or adjacent to, a derivative of said analyte. 207. The method of embodiment 201, wherein said probe ligates to, or adjacent to, said analyte. 208. The method of embodiment 201, wherein said probe ligates to, or adjacent to, a derivative of said analyte. 209. The method of embodiment 206 or 208, wherein said derivative is an amplification product of said analyte. 210. The method of embodiment 206 or 208, wherein said derivative is an extension product of said analyte. 211. The method of embodiment 206 or 208, wherein said derivative is a circularization product of said analyte. 212. The method of embodiment 199, wherein said probe comprises an optical moiety. 213. The method of embodiment 212, wherein said probe comprises a fluorescent dye. 214. The method of embodiment 213, wherein said one or more reagents comprises an additional probe configured to detect an additional analyte, wherein said additional probe comprises an additional fluorescent dye. 215. The method of embodiment 214, wherein said fluorescent dye and said additional fluorescent dye are different. 216. The method of embodiment 214, wherein said fluorescent dye and said additional fluorescent dye are the same. 217. The method of embodiment 214, wherein said probe and said additional probe are different. 218. The method of embodiment 214, wherein said probe and said additional probe are the same. 219. The method of embodiment 214, wherein said analyte and said additional analyte are different types of analyte. 220. The method of embodiment 214, wherein said analyte and said additional analyte are the same type of analyte. 221. The method of embodiment 212, further comprising, subsequent to (c), (i) delivering an additional probe to said cell or tissue sample, wherein said additional probe is configured to detect an additional analyte, and (ii) using said detector to detect said additional analyte of said cell or tissue sample. 222. The method of embodiment 184 or embodiment 185, wherein (c) comprises identifying (1) an identity and (2) a location of said analyte in said cell or tissue sample. 223. The method of embodiment 222, wherein said location is a two dimensional (2D) location. 224. The method of embodiment 222, wherein said location is a three dimensional (3D) location. 225. The method of embodiment 222, wherein said identity comprises a sequence of said analyte. 226. The method of embodiment 222, wherein said identity comprises a type of said analyte. 227. The method of embodiment 184 or embodiment 185, wherein said substrate is substantially planar. 228. The method of embodiment 222, wherein said substrate is patterned. 229. The method of embodiment 228, wherein said substrate is patterned via surface chemistry. 230. The method of embodiment 184 or embodiment 185, wherein said substrate has a shape, wherein said shape comprises a regular polygon or an irregular polygon. 231. The method of embodiment 230, wherein said shape comprises said regular polygon. 232. The method of embodiment 231, wherein said regular polygon comprises a rectangle. 233. The meth of embodiment 184 or embodiment 185, wherein said substrate has a shape, wherein said shape comprises a circle or an oval. 234. The method of embodiment 184 or embodiment 185, wherein said substrate comprises an array of a plurality of individually addressable locations, wherein in (a) said cell or tissue sample is immobilized to one or more individually addressable locations of said plurality of individually addressable locations. 235. The method of embodiment 234, wherein said array comprises at least 1,000 individually addressable locations. 236. The method of embodiment 234, wherein said array comprises at least 100,000 individually addressable locations. 237. The method of embodiment 234, wherein said array comprises at least 10,000,000 individually addressable locations. 238. The method of embodiment 234, wherein said array comprises at least 50,000,000,000 individually addressable locations. 239. The method of embodiment 184 or embodiment 185, wherein (a) comprises providing said substrate comprising a plurality of cells or tissue samples thereon. The method of embodiment 239, wherein said plurality of cells or tissue samples comprises at least about 100 to at least about 1,000,000 cells or tissue samples.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/173,228, filed Apr. 9, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method, comprising:
(a) immobilizing a plurality of beads to individually addressable locations of a first substrate to provide a barcoded substrate, wherein the plurality of beads comprises oligonucleotide molecules coupled thereto, wherein the oligonucleotide molecules comprise barcode sequences;
(b) sequencing the oligonucleotide molecules on the barcoded substrate to generate indexed data that indexes the individually addressable locations of the first substrate with the barcode sequences of the oligonucleotide molecules, to provide an indexed, barcoded substrate, wherein the sequencing comprises rotating the barcoded substrate during dispensing of sequencing reagents to the barcoded substrate or during imaging of the barcoded substrate;
(c) loading a biological sample to the indexed, barcoded substrate to tag analytes in the biological sample using the oligonucleotide molecules, to generate spatially tagged analytes, wherein the spatially tagged analytes comprise the barcode sequences or derivatives thereof;
(d) immobilizing the spatially tagged analytes, or derivatives thereof, on a second substrate;
(e) sequencing the spatially tagged analytes, or derivatives thereof, on the second substrate to generate sequencing data, wherein the sequencing comprises rotating the second substrate during dispensing of sequencing reagents to the second substrate or during imaging of the second substrate; and
(f) generating spatial data of the analytes of the biological sample using the sequencing data and the indexed data.

2. The method of claim 1, wherein a bead of the plurality of beads comprises at least 100,000 oligonucleotide molecules, and wherein the at least 100,000 oligonucleotide molecules comprise a barcode sequence of the barcode sequences that is common and unique to the bead amongst the plurality of beads.

3. The method of any one of claims 1-2, wherein an oligonucleotide molecule of the oligonucleotide molecules comprises a capture sequence, wherein the capture sequence hybridizes with a sequence of an analyte, or derivative thereof, of the analytes.

4. The method of claim 3, wherein the capture sequence is selected from the group consisting of: a polyT sequence, a polyG sequence, a targeted mRNA sequence, a targeted gDNA sequence, a random n-mer sequence, and a probe sequence.

5. The method of any one of claims 1-4, wherein the biological sample comprises a tissue.

6. The method of any one of claims 1-5, wherein the analytes comprise mRNA, DNA, or proteins.

7. The method of any one of claims 1-6, wherein the first substrate comprises at least 1,000,000 individually addressable locations.

8. The method of any one of claims 1-7, wherein the plurality of beads is immobilized to the individually addressable locations via electrostatic interactions.

9. The method of any one of claims 1-8, wherein the spatially tagged analytes, or derivatives thereof, are coupled to a second plurality of beads, which second plurality of beads are immobilized to second individually addressable locations of the second substrate.

10. The method of claim 9, wherein a second bead of the second plurality of beads comprises a colony of a spatially tagged analyte, or derivative thereof, of the spatially tagged analytes, or derivatives thereof.

11. The method of claim 10, wherein the second bead comprises at least 100,000 copies of the spatially tagged analyte, or derivative thereof.

12. The method of any one of claims 1-11, wherein the sequencing in (b) comprises sequencing by synthesis, wherein the sequencing by synthesis comprises providing distinct nucleotide flows to the first substrate, wherein a distinct nucleotide flow of the distinct nucleotide flows comprises a single-base nucleotide mixture.

13. The method of claim 12, wherein the single-base nucleotide mixture comprises a mixture of labeled nucleotides and non-labeled nucleotides of a single base.

14. The method of any one of claims 12-13, wherein the single-base nucleotide mixture comprises non-terminated nucleotides.

15. The method of any one of claims 1-14, wherein the sequencing in (b) comprises rotating the barcoded substrate during dispensing of sequencing reagents, rotating the barcoded substrate during imaging of the barcoded substrate, or rotating the barcoded substrate during dispensing of sequencing reagents and rotating the barcoded substrate during imaging of the barcoded substrate.

16. The method of any one of claims 1-15, wherein the sequencing in (e) comprises sequencing by synthesis.

17. The method of any one of claims 1-16, wherein the sequencing in (e) comprises rotating the second substrate during dispensing of sequencing reagents, rotating the second substrate during imaging of the second substrate, or rotating the second substrate during dispensing of sequencing reagents and rotating the second substrate during imaging of the second substrate.

18. The method of any one of claims 1-17, wherein the first substrate or the second substrate is substantially planar.

19. The method of any one of claims 1-18, further comprising releasing the oligonucleotide molecules from the plurality of beads.

20. The method of claim 19, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene.

21. The method of any one of claims 1-20, further comprising fixing the biological sample.

22. The method of any one of claims 1-21, further comprising permeabilizing the biological sample.

23. A method, comprising:
(a) providing (1) a first substrate comprising a plurality of beads immobilized thereto, wherein the plurality of beads comprises oligonucleotide molecules coupled thereto, wherein the oligonucleotide molecules comprise barcode sequences, and (2) indexed data comprising identities and locations of the barcode sequences on the first substrate;
(b) loading a biological sample to the first substrate to tag analytes in the biological sample using the oligonucleotide molecules, to generate spatially tagged analytes, wherein the spatially tagged analytes comprise the barcode sequences or derivatives thereof;
(c) immobilizing the spatially tagged analytes, or derivatives thereof, on a second substrate;
(d) sequencing the spatially tagged analytes, or derivatives thereof, on the second substrate to generate sequencing data, wherein the sequencing comprises rotating the second substrate during dispensing of sequencing reagents to the second substrate or during imaging of the second substrate; and
(e) generating spatial data of the analytes of the biological sample using the sequencing data and the indexed data.

24. The method of claim 23, wherein a bead of the plurality of beads comprises at least 100,000 oligonucleotide molecules, and wherein the at least 100,000 oligonucleotide molecules comprise a barcode sequence of the barcode sequences that is common and unique to the bead amongst the plurality of beads.

25. The method of any one of claims 23-24, wherein an oligonucleotide molecule of the oligonucleotide molecules comprises a capture sequence, wherein the capture sequence hybridizes with a sequence of an analyte, or derivative thereof, of the analytes.

26. The method of claim 25, wherein the capture sequence is selected from the group consisting of: a polyT sequence, a polyG sequence, a targeted mRNA sequence, a targeted gDNA sequence, a random n-mer sequence, and a probe sequence.

27. The method of any one of claims 23-26, wherein the biological sample comprises a tissue.

28. The method of any one of claims 23-27, wherein the analytes comprise mRNA, DNA, or proteins.

29. The method of any one of claims 23-28, wherein the first substrate comprises at least 1,000,000 individually addressable locations, wherein the plurality of beads are immobilized to the at least 1,000,000 individually addressable locations.

30. The method of claim 29, wherein the first substrate comprises at least 1,000,000,000 individually addressable locations.

31. The method of any one of claims 23-30, wherein the plurality of beads is immobilized to the at least 1,000,000 individually addressable locations via electrostatic interactions.

32. The method of any one of claims 23-31, wherein the spatially tagged analytes, or derivatives thereof, are coupled to a second plurality of beads, which second plurality of beads are immobilized to second individually addressable locations of the second substrate.

33. The method of claim 32, wherein a second bead of the second plurality of beads comprises a colony of a spatially tagged analyte, or derivative thereof, of the spatially tagged analytes, or derivatives thereof.

34. The method of claim 33, wherein the second bead comprises at least 100,000 copies of the spatially tagged analyte, or derivative thereof.

35. The method of any one of claims 23-34, wherein the sequencing in (d) comprises sequencing by synthesis, and wherein the sequencing by synthesis comprises providing distinct nucleotide flows to the second substrate, wherein a distinct nucleotide flow of the distinct nucleotide flows comprises a single-base nucleotide mixture.

36. The method of claim 35, wherein the single-base nucleotide mixture comprises a mixture of labeled nucleotides and non-labeled nucleotides of a single base.

37. The method of any one of claims 35-36, wherein the single-base nucleotide mixture comprises non-terminated nucleotides.

38. The method of any one of claims 23-37, wherein the sequencing in (d) comprises rotating the second substrate during dispensing of sequencing reagents, rotating the second substrate during imaging of the second substrate, or rotating the second substrate during dispensing of sequencing reagents and rotating the second substrate during imaging of the second substrate.

39. The method of any one of claims 23-38, wherein the first substrate or the second substrate is substantially planar.

40. The method of any one of claims 23-39, further comprising releasing the oligonucleotide molecules from the plurality of beads.

41. The method of claim 40, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene.

42. The method of any one of claims 23-41, further comprising fixing the biological sample.

43. The method of any one of claims 23-42, further comprising permeabilizing the biological sample.

44. A method, comprising:
(a) sequencing a first substrate comprising a plurality of beads immobilized thereto, wherein the plurality of beads comprises oligonucleotide molecules coupled thereto, wherein the oligonucleotide molecules comprise barcode sequences, to generate indexed data comprising identities and locations of the barcode sequences on the first substrate;
(b) loading a biological sample to the first substrate to tag analytes in the biological sample using the oligonucleotide molecules, to generate spatially tagged analytes, wherein the spatially tagged analytes comprise the barcode sequences or derivatives thereof;
(c) immobilizing the spatially tagged analytes, or derivatives thereof, on a second substrate; and
(d) sequencing the second substrate to generate sequencing data,
wherein the first substrate or the second substrate or both are rotated during the sequencing in (a) or (d), respectively.

45. The method of claim 44, wherein a bead of the plurality of beads comprises at least 100,000 oligonucleotide molecules, and wherein the at least 100,000 oligonucleotide molecules comprise a barcode sequence of the barcode sequences that is common and unique to the bead amongst the plurality of beads.

46. The method of any one of claims 44-45, wherein an oligonucleotide molecule of the oligonucleotide molecules comprises a capture sequence, wherein the capture sequence hybridizes with a sequence of an analyte, or derivative thereof, of the analytes.

47. The method of claim 46, wherein the capture sequence is selected from the group consisting of: a polyT sequence, a polyG sequence, a targeted mRNA sequence, a targeted gDNA sequence, a random n-mer sequence, and a probe sequence.

48. The method of any one of claims 44-47, wherein the biological sample comprises a tissue.

49. The method of any one of claims 44-48, wherein the analytes comprise mRNA, DNA, or proteins.

50. The method of any one of claims 44-49, wherein the first substrate comprises at least 1,000,000 individually addressable locations immobilizing the plurality of beads.

51. The method of claim 50, wherein the first substrate comprises at least 1,000,000,000 individually addressable locations immobilizing the plurality of beads.

52. The method of claim 51, wherein the plurality of beads is immobilized to the at least 1,000,000 individually addressable locations via electrostatic interactions.

53. The method of any one of claims 44-52, wherein the spatially tagged analytes, or derivatives thereof, are coupled to a second plurality of beads, which second plurality of beads are immobilized to second individually addressable locations of the second substrate.

54. The method of claim 53, wherein the second substrate comprises at least 1,000,000,000 second individually addressable locations.

55. The method of any one of claims 53-54, wherein a second bead of the second plurality of beads comprises a colony of a spatially tagged analyte, or derivative thereof, of the spatially tagged analytes, or derivatives thereof.

56. The method of claim 55, wherein the second bead comprises at least 100,000 copies of the spatially tagged analyte, or derivative thereof.

57. The method of any one of claims 44-56, wherein the sequencing in (a) comprises sequencing by synthesis.

58. The method of claim 57, wherein the sequencing by synthesis comprises providing distinct nucleotide flows to the first substrate, wherein a distinct nucleotide flow of the distinct nucleotide flows comprises a single-base nucleotide mixture.

59. The method of claim 58, wherein the single-base nucleotide mixture comprises a mixture of labeled nucleotides and non-labeled nucleotides of a single base.

60. The method of any one of claims 58-59, wherein the single-base nucleotide mixture comprises non-terminated nucleotides.

61. The method of any one of claims 44-60, wherein the sequencing in (a) comprises rotating the first substrate during dispensing of sequencing reagents, rotating the first substrate during imaging of the first substrate, or rotating the first substrate during dispensing of sequencing reagents and rotating the first substrate during imaging of the first substrate.

62. The method of any one of claims 44-61, wherein the sequencing in (d) comprises sequencing by synthesis.

63. The method of any one of claims 44-62, wherein the sequencing in (d) comprises rotating the second substrate during dispensing of sequencing reagents, rotating the second substrate during imaging of the second substrate, rotating the second substrate during dispensing of sequencing reagents and rotating the second substrate during imaging of the second substrate.

64. The method of any one of claims 44-63, wherein the first substrate or the second substrate is substantially planar.

65. The method of any one of claims 44-64, further comprising releasing the oligonucleotide molecules from the plurality of beads.

66. The method of claim 65, wherein the releasing comprises one or more of the following: (i) providing an enzyme mix comprising an enzyme configured to cleave or digest a cleavage site, wherein the oligonucleotide molecules comprises the cleavage site; (ii) providing biotin, wherein the oligonucleotide molecules are conjugated to a desthiobiotin moiety, wherein the plurality of beads comprises a streptavidin moiety bound to the desthiobiotin moiety; (iii) providing an enzyme mix comprising an enzyme configured to cleave or digest a cleavage site and UV light, wherein the oligonucleotide molecules comprises azobenzene and the cleavage site; and (iv) providing UV light, wherein the oligonucleotide molecules are conjugated to azobenzene, wherein the plurality of beads comprises an alpha-cyclodextrine (a-CD) moiety bound to the azobenzene.

67. The method of any one of claims 44-66, further comprising fixing the biological sample.

68. The method of any one of claims 44-67, further comprising permeabilizing the biological sample.

69. The method of any one of claims 44-68, further comprising generating spatial data of the analytes in the biological sample using at least the indexed data and the sequencing data.

70. A system, comprising:
a sequencing platform configured to (i) address individually addressable locations of substrates and (ii) rotate the substrates during dispensing of sequencing reagents to the substrates or during imaging of the substrates or during both; and
a first substrate comprising a plurality of beads immobilized to a plurality of individually addressable locations on the substrate, wherein the plurality of beads comprises oligonucleotide molecules coupled thereto, wherein the oligonucleotide molecules comprise barcode sequences.

71. The system of claim 70, further comprising indexed data comprising identities and locations of the barcode sequences on the first substrate.

72. The system of any one of claims 70-71, further comprising a second substrate comprising a second plurality of individually addressable locations configured to immobilize a second plurality of beads.

73. The system of claim 72, further comprising the second plurality of beads.

74. The system of any one of claims 70-73, further comprising a reagent configured to release the oligonucleotide molecules from the plurality of beads.

75. The system of claim 74, wherein the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene.

76. The system of any one of claims 70-75, further comprising sequencing reagents.

77. The system of claim 76, wherein the sequencing reagents comprise single-base nucleotide mixtures.

78. The system of claim 77, wherein a single-base nucleotide mixture of the single-base nucleotide mixtures comprises a mixture of labeled nucleotides and non-labeled nucleotides of a single base.

79. The system of any one of claims 76-77, wherein a single-base nucleotide mixture of the single-base nucleotide mixtures comprises non-terminated nucleotides.

80. The system of any one of claims 70-79, further comprising amplification reagents.

81. The system of any one of claims 70-80, further comprising a biological sample.

82. The system of claim 81, wherein the biological sample is a tissue.

83. The system of any one of claims 81-82, wherein the biological sample is fixed.

84. The system of any one of claims 81-83, wherein the biological sample is permeabilized.

85. The system of any one of claims 81-84, wherein the biological sample is loaded on the first substrate.

86. The system of any one of claims 70-85, wherein a bead of the plurality of beads comprises at least 100,000 oligonucleotide molecules, and wherein the at least 100,000 oligonucleotide molecules comprise a barcode sequence of the barcode sequences that is common and unique to the bead amongst the plurality of beads.

87. The system of any one of claims 70-86, wherein an oligonucleotide molecule of the oligonucleotide molecules comprises a capture sequence, wherein the capture sequence is configured to hybridize with a sequence of an analyte, or derivative thereof, of a biological sample.

88. The system of claim 87, wherein the capture sequence is selected from the group consisting of: a polyT sequence, a polyG sequence, a targeted mRNA sequence, a targeted gDNA sequence, a random n-mer sequence, and a probe sequence.

89. The system of any one of claims 70-88, wherein the first substrate comprises at least 1,000,000 individually addressable locations immobilizing the plurality of beads.

90. The system of claim 89, wherein the first substrate comprises at least 1,000,000,000 individually addressable locations immobilizing the plurality of beads.

91. The system of any one of claims 70-90, wherein the plurality of beads is immobilized to the individually addressable locations via electrostatic interactions.

92. The system of any one of claims 70-91, wherein the sequencing platform is configured to perform sequencing by synthesis on the substrates.

93. The system of any one of claims 70-92, wherein the first substrate is substantially planar.

94. A kit, comprising:
a first substrate comprising a plurality of beads immobilized to a plurality of individually addressable locations on the substrate, wherein the plurality of beads comprises oligonucleotide molecules coupled thereto, wherein the oligonucleotide molecules comprise barcode sequences;
indexed data comprising identities and locations of the barcode sequences on the first substrate; and
a second substrate comprising a second plurality of individually addressable locations configured to immobilize a second plurality of beads.

95. The kit of claim 94, further comprising the second plurality of beads not immobilized to the second substrate.

96. The kit of any one of claims 94-95, further comprising a reagent configured to release the oligonucleotide molecules from the plurality of beads.

97. The kit of claim 96, wherein the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene.

98. The kit of any one of claims 94-97, further comprising sequencing reagents.

99. The kit of claim 98, wherein the sequencing reagents comprise single-base nucleotide mixtures.

100. The kit of claim 99, wherein a single-base nucleotide mixture of the single-base nucleotide mixtures comprises a mixture of labeled nucleotides and non-labeled nucleotides of a single base.

101. The kit of any one of claims 99-100, wherein a single-base nucleotide mixture of the single-base nucleotide mixtures comprises non-terminated nucleotides.

102. The kit of any one of claims 94-101, further comprising amplification reagents.

103. The kit of any one of claims 94-102, wherein a bead of the plurality of beads comprises at least 100,000 oligonucleotide molecules, and wherein the at least 100,000 oligonucleotide molecules comprise a barcode sequence of the barcode sequences that is common and unique to the bead amongst the plurality of beads.

104. The kit of any one of claims 94-103, wherein an oligonucleotide molecule of the oligonucleotide molecules comprises a capture sequence, wherein the capture sequence is configured to hybridize with a sequence of an analyte, or derivative thereof, of a biological sample.

105. The kit of claim 104, wherein the capture sequence is selected from the group consisting of: a polyT sequence, a polyG sequence, a targeted mRNA sequence, a targeted gDNA sequence, a random n-mer sequence, and a probe sequence.

106. The kit of any one of claims 94-105, wherein the first substrate comprises at least 1,000,000 individually addressable locations.

107. The kit of claim 106, wherein the first substrate comprises at least 1,000,000,000 individually addressable locations.

108. The kit of any one of claims 94-107, wherein the plurality of beads is immobilized to the plurality of individually addressable locations via electrostatic interactions.

109. The kit of any one of claims 94-108, wherein the first substrate or the second substrate is substantially planar.

110. The kit of any one of claims 94-109, wherein the first substrate and the second substrate are substantially identical in size.

111. A kit, comprising:
a substrate comprising a plurality of beads immobilized to a plurality of individually addressable locations on the substrate, wherein the plurality of beads comprises oligonucleotide molecules coupled thereto, wherein the oligonucleotide molecules comprise barcode sequences,
wherein the oligonucleotide molecules are releasable from the plurality of beads by one or more of the release mechanisms selected from the group consisting of:
(a) providing an enzyme mix comprising an enzyme configured to cleave or digest a cleavage site, wherein the oligonucleotide molecules comprises the cleavage site;
(b) providing biotin, wherein the oligonucleotide molecules are conjugated to a desthiobiotin moiety, wherein the bead comprises a streptavidin moiety bound to the desthiobiotin moiety;
(c) providing an enzyme mix comprising an enzyme configured to cleave or digest a cleavage site and UV light, wherein the oligonucleotide molecules comprises azobenzene and the cleavage site; and
(d) providing UV light, wherein the oligonucleotide molecules are conjugated azobenzene, wherein the plurality of beads comprises an alpha-cyclodextrine (a-CD) moiety bound to the azobenzene.

112. The kit of claim 111, further comprising indexed data comprising identities and locations of the barcode sequences on the substrate.

113. The kit of any one of claims 111-112, further comprising a second substrate comprising a second plurality of individually addressable locations configured to immobilize a second plurality of beads.

114. The kit of claim 113, further comprising the second plurality of beads.

115. The kit of any one of claims 111-114, further comprising a reagent configured to release the oligonucleotide molecules from the plurality of beads.

116. The kit of claim 115, wherein the reagent comprises one or more of: (i) an enzyme mix comprising an enzyme configured to cleave or digest a cleavage site, wherein the oligonucleotide molecules comprises the cleavage site; (ii) biotin, wherein the oligonucleotide molecules are conjugated to a desthiobiotin moiety, wherein the plurality of beads comprises a streptavidin moiety bound to the desthiobiotin moiety; (iii) an enzyme mix comprising an enzyme configured to cleave or digest a cleavage site and UV light, wherein the oligonucleotide molecules comprises azobenzene and the cleavage site; and (iv) a light source configured to provide UV light, wherein the oligonucleotide molecules are conjugated to azobenzene, wherein the plurality of beads comprises an alpha-cyclodextrine (a-CD) moiety bound to the azobenzene.

117. The kit of any one of claims 111-116, further comprising sequencing reagents.

118. The kit of claim 117, wherein the sequencing reagents comprise single-base nucleotide mixtures.

119. The kit of claim 118, wherein a single-base nucleotide mixture of the single-base nucleotide mixtures comprises a mixture of labeled nucleotides and non-labeled nucleotides of a single base.

120. The kit of any one of claims 118-119, wherein a single-base nucleotide mixture of the single-base nucleotide mixtures comprises non-terminated nucleotides.

121. The kit of any one of claims 111-120, further comprising amplification reagents.

122. The kit of any one of claims 111-121, wherein a bead of the plurality of beads comprises at least 100,000 oligonucleotide molecules, and wherein the at least 100,000 oligonucleotide molecules comprise a barcode sequence of the barcode sequences that is common and unique to the bead amongst the plurality of beads.

123. The kit of any one of claims 111-122, wherein an oligonucleotide molecule of the oligonucleotide molecules comprises a capture sequence, wherein the capture sequence is configured to hybridize with a sequence of an analyte, or derivative thereof, of a biological sample.

124. The kit of claim 123, wherein the capture sequence is selected from the group consisting of: a polyT sequence, a polyG sequence, a targeted mRNA sequence, a targeted gDNA sequence, a random n-mer sequence, and a probe sequence.

125. The kit of any one of claims 111-124, wherein the substrate comprises at least 1,000,000 individually addressable locations.

126. The kit of claim 125, wherein the substrate comprises at least 1,000,000,000 individually addressable locations.

127. The kit of any one of claims 111-126, wherein the plurality of beads is immobilized to the individually addressable locations via electrostatic interactions.

128. The kit of any one of claims 111-127, wherein the substrate is substantially planar.

129. A method, comprising:
(a) providing a substrate comprising a cell or tissue sample thereon;
(b) subjecting said substrate to rotation to distribute one or more reagents to said cell or tissue sample; and
(c) using a detector in optical communication with said cell or tissue sample on said substrate to detect an analyte of said cell or tissue sample.

130. A method, comprising:
(a) providing a substrate comprising a cell or tissue sample thereon;
(b) distributing one or more reagents to said cell or tissue sample; and
(c) using a detector in optical communication with said cell or tissue sample on said substrate to detect an analyte of said cell or tissue sample, during rotation of said substrate.

131. The method of claim 129 or claim 130, wherein said analyte is within said cell or tissue sample.

132. The method of claim 131, wherein said analyte comprises a nucleic acid molecule, a polypeptide molecule, a protein molecule, a carbohydrate molecule, a lipid molecule, any derivative thereof, or any combination thereof.

133. The method of any one of claims 129-132, wherein said one or more reagents comprises a probe configured to detect said analyte.

134. The method of claim 133, wherein said probe comprises one or more nucleotides or one or more nucleic acid molecules.

135. The method of claim 134, wherein said probe comprises a padlock probe.

136. The method of any one of claims 134-135, wherein said probe comprises a barcode sequence.

137. The method of any one of claims 134-136, wherein said probe hybridizes or ligates to, or adjacent to, said analyte or a derivative thereof.

138. The method of claim 137, wherein said derivative is an amplification product, extension product, or circularization product of said analyte.

139. The method of any one of claims 133-138, wherein said probe comprises an optical moiety.

140. The method of any one of claims 129-139, wherein (c) comprises identifying (1) an identity and (2) a location of said analyte in said cell or tissue sample.

141. The method of claim 140, wherein said location is a two dimensional (2D) location or a three dimensional (3D) location.

142. The method of any one of claims 140-141, wherein said identity comprises a sequence of said analyte or a type of said analyte.

143. The method of any one of claims 129-142, wherein said substrate is substantially planar.

144. The method of any one of claims 129-143, wherein said substrate is patterned.

145. The method of claim 144, wherein said substrate is patterned via surface chemistry.

146. The method of any one of claims 129-145, wherein said substrate comprises an array of a plurality of individually addressable locations, wherein in (a) said cell or tissue sample is immobilized to one or more individually addressable locations of said plurality of individually addressable locations.

147. The method of claim 146, wherein said array comprises at least 1,000, at least 100,000, at least 10,000,000, or at least 50,000,000,000 individually addressable locations.
